(19) United States
(12) Patent Application Publication
(10) Pub. No.: US 2022/0020455 A1
(43) Pub. Date: Jan. 20, 2022

(54) POINT-OF-CARE DIAGNOSTIC INSTRUMENT WORKFLOW

(71) Applicant: Talis Biomedical Corporation, Menlo Park, CA (US)

(72) Inventors: Thomas H. CAULEY, III, Redwood City, CA (US); Marc VALER SERRA, Menlo Park, CA (US)

(21) Appl. No.: 16/928,994

(22) Filed: Jul. 14, 2020

(51) Int. Cl. G16H 10/40; G16H 50/20; G16H 70/60; G16H 30/40; G16H 30/20

(52) U.S. Cl. CPC G16H 10/40; G16H 50/20; G16H 30/20; G16H 30/40; G16H 70/60

(57) ABSTRACT

Methods are provided for operating an instrument to test samples suspected of containing a target pathogen in a near patient point of care environment. One embodiment of the method includes adding an identifying mark to a point of care cartridge and inserting the cartridge into an instrument. Additionally, interaction with a graphical user interface on the instrument can initiate the test sequence or eject the cartridge.

### INCORPORATION BY REFERENCE

All publications and patent applications mentioned in this specification are herein incorporated by reference to the same extent as if each individual publication or patent application was specifically and individually indicated to be incorporated by reference.

### STATEMENT AS TO FEDERALLY SPONSORED RESEARCH

The various embodiments of the inventions described herein were not made with government support.

### FIELD

Workflows to assist in the collection and processing of samples in compliance with a waived Clinical Laboratory Improvement Amendments test (“CLIA waived test”) performed using an integrated diagnostic cartridge and instrument in a near patient point of care environment.

### BACKGROUND

Modern advancements in molecular diagnostic testing technology have enabled the development of point of care diagnostic systems for accurately detecting and diagnosing infectious diseases in near patient environments, e.g. a physician's office or clinic, which is at the time and place of patient care. However, existing commercially available point of care diagnostic systems pose challenges relating to their ease of use including, but not limited to, cumbersome physician or clinical workflows coupled with an unknown skill level for system users, sample handling requirements, and complicated record keeping systems. Many of these point of care systems have adopted and modified existing patient sample and identification infrastructure from central laboratories and hospitals, prompting unnecessarily complex testing procedures and record keeping systems.

Typically, existing point of care systems require multiple preparation steps between the time a biological sample is collected from a patient to when the sample is tested using a diagnostic system. Such preparation steps may include pretreatment of swab or liquid samples and multiple handling steps involving sample custody. Barcodes and other machine readable codes have been used in central laboratories and other large medical facilities to track a patient ID and other patient information through preliminary preparation steps and the final test result. Despite operating in a point of care setting, various diagnostic systems may require a user to scan one or more barcodes throughout the preparation steps prior to inserting the sample into the diagnostic system and beginning the test.

The inclusion of numerous sample handling steps results in a tedious and lengthy workflow. An exemplary point of care diagnostic instrument workflow may require a user to perform the following steps prior to inserting the sample and/or consumable into the diagnostic system: (1) scanning a barcode on a login security card and/or entering login information, e.g. a password; (2) scanning a barcode located on a sample collection container; (2) scanning a barcode located on a disposable diagnostic consumable; and (3) scanning a patient ID and/or entering the patient information manually. Each unnecessary interaction between the user and the diagnostic system or between the user and the patient sample extends the length of time to obtain a test result, introduces a potential for error and increases the likelihood of an erroneous or invalid test result.

Therefore, despite the existence of some point of care diagnostic systems, a need exists for a simplified point of care system workflows that enable simple operational procedures for the end user and achieves clinically accurate results without a significant risk of error.

### SUMMARY OF THE DISCLOSURE

In general, in one embodiment, a method of operating an instrument for testing a sample suspected of containing a target pathogen includes: (1) loading the sample suspected of containing the target pathogen into a sample port assembly of a cartridge; (2) adding an identifying mark to a patient label area of the cartridge; (3) inserting the cartridge into an opening of the instrument until the cartridge is positioned within the instrument with the identifying mark within a field of view of a label imaging camera; (4) observing on a graphical user interface of the instrument an indication of a type of test to be performed on the cartridge and an image of the identifying mark on the patient label area of the cartridge; (5) interacting with the graphical user interface of the instrument to eject the cartridge if the image of the identifying mark or the indication of the type of test is incorrect; and (6) removing the cartridge from the opening of the instrument after the cartridge is automatically ejected from the opening.

This and other embodiments can include one or more of the following features. The method can further include observing an error message on the graphical user interface before or during the removing step. The method can further include observing an image of the identifying mark and an indication of a presence, an absence or a quantity of the target pathogen in the sample on the graphical user interface before or during the removing step. The method can further include automatically initiating a testing protocol when a predetermined time period has elapsed after completing the inserting the cartridge step. The predetermined time period can be less than 2 minutes, less than one minute, less than 30 seconds, or less than 10 seconds. The method can further include interacting with the graphical user interface to initiate a testing protocol after the observing step has been performed. The adding an identifying mark step can further include affixing a printed label or a printed machine readable label to the patient label area. The patient label area can be adjacent to the sample port assembly. The adding an identifying mark step can further include handwriting sample identifying information in the patient label area. The method can further include touching the graphical user interface to enter a security code after performing the observing step. The method can further include observing on the graphical user interface a progress timer of the testing protocol or a listing of one or more previously testing protocol results. The method can further include initiating at least one cartridge verification test without any user interaction with the instrument after performing the inserting the cartridge step. The method can further include observing on the graphical user interface of the instrument the identifying mark without touching the graphical user interface of the instrument or performing any other user interaction to contact the instrument while or prior to performing the removing the cartridge step. After the insertion step, the instrument can automatically perform a nucleic acid amplification process to produce a result that contains an indication of a presence, an absence or a quantity of the target pathogen in the sample suspected of containing the target pathogen without touching the graphical user interface of the instrument or otherwise interacting with the instrument. The method can further include observing an image, an icon or a glyph on the graphical user interface indicating a result of a testing protocol performed on the sample in the cartridge during, after, or before the removing step. The step of observing the image, the icon, or the glyph on the graphical user display can be performed less than 60 min., 25 min, less than 20 min, less than 15 min, or less than 10 min after performing the inserting a cartridge into the instrument step. The observing step can further include waiting for completion of a testing protocol.

In general, in one embodiment, a method of testing a sample suspected of containing a target pathogen includes: (1) inserting the sample suspected of containing the target pathogen into a point of care cartridge; (2) placing an identifying mark on a patient label section of the point of care cartridge; (3) inserting the point of care cartridge into an opening of a point of care instrument until the patient label section of the point of care cartridge is within a field of view of a label imaging camera within an interior portion of the point of care instrument; (4) observing on a graphical user interface of the point of care instrument an image of the patient label section captured by the label imaging camera; and (5) performing only a single interaction with the point of care instrument to observe on the graphical user interface, adjacent to the image of the patient label section, a single indicator representing a result of a testing sequence indicating a presence of the target pathogen, an absence of the target pathogen, or a quantity of the target pathogen in the sample.

This and other embodiments can include one or more of the following features. The placing step can further include handwriting on the patient label section to identify the sample. The placing step can further include affixing a printed label in the patient label section to identify the sample. The placing step can further include marking a pre-printed box, circle, geometric shape, or area in the patient label section indicating a sample type contained in the point of care cartridge. A time delay of less than 15 minutes can separate the observing step from the performing step. The step of performing a single interaction can further include entering a security code into the graphical user interface to permit interaction with the point of care instrument. After the inserting step, the point of care cartridge can be substantially within the interior of the point of care instrument. The performing only a single interaction step can be undertaken after observing that the point of care cartridge is ejected from the point of care instrument. The single indicator can represent a positive test result or a negative test result. The single indicator for the positive test result can appear in red in the graphical user interface and the single indicator for the negative test result can appear in green in the graphical user interface. The single indicator can be an image, an icon, or glyph. The single indicator can include a number of text characters. The single indicator representing a result can further include an image, an icon, or a glyph for a presence of the pathogen or an absence of the pathogen. The single indicator can represent a result for two or more tests performed on the point of care cartridge. The single indicator can represent a negative presence of all target pathogens from the two or more tests or the single indicator can represent a positive presence of at least one target pathogen from the two or more tests. The method can further include interacting with the graphical user interface to display individual results of each of the two or more tests performed on the point of care cartridge. The method can further include performing the inserting the sample step, the placing the identifying mark step, the inserting the point of care cartridge step, the observing on the graphical user interface step and the performing only the single interaction with the point of care instrument step on each one of a plurality of point of care cartridges to produce a plurality of the single indicator representing the result of the testing sequence performed on each one of a plurality of the point of care cartridges. The method can further include interacting with the graphical user interface to scroll through the plurality of the single indictor representing the result of the testing sequence performed on each one of the plurality of the point of care cartridges. The method can further include preventing the display of individual test results on the graphical user interface after a time interval. The method can further include allowing the display of individual test results on the graphical user interface after entering a security code using the graphical user interface.

In general, in one embodiment, a method of operating an instrument for testing a liquid sample suspected of containing a target pathogen includes: (1) loading the liquid sample suspected of containing the target pathogen into a sample port of a cartridge; (2) adding an identifying mark to the cartridge; and (3) inserting the cartridge into an instrument configured to perform a test in the cartridge to produce a result that contains an indication of a presence, an absence or a quantity of the target pathogen in the sample suspected of containing the target pathogen; wherein inserting the cartridge into the instrument causes the instrument to: (1) confirm a quantity of the sample in a loading chamber of the liquid sample suspected of containing the target pathogen; (2) confirm the cartridge is ready for use; (3) complete a cartridge-to-instrument interface test; and (4) display an image of the identifying mark on the cartridge on a graphical user interface of the instrument.

This and other embodiments can include one or more of the following features. The method can further include causing the instrument to initiate the test in the cartridge after displaying the image of the identifying mark on the graphical user interface for a predetermined time interval of less than 90 seconds. The method can further include causing the instrument to eject the cartridge if the step to confirm the quantity of the sample in the loading chamber indicates an insufficient quantity of the sample or the step to confirm the cartridge is ready for use indicates the cartridge is not ready for use or the step to complete a cartridge-to-interface test indicates an unsatisfactory cartridge-to-instrument interface. The liquid sample can have a volume between 0.2 ml and 5 ml, inclusive. The volume of the liquid sample can be between 0.5 ml and 1.5 ml, inclusive. The volume of the liquid sample can be approximately 1 ml. The liquid sample can be urine, blood, sputum, saliva, or oral fluids. The liquid sample can be a suspension released from a swab collected from a patient. Loading the sample can further include sealing the sample port. The identifying mark can be handwritten. The identifying mark can be a barcode. The identifying mark can identify a patient from which the sample is acquired. The patient can be identified by name, ID number and/or date-of-birth. The identifying mark can further include a sample type. The sample type can be selected from the group consisting of urine, blood, sputum, saliva, oral fluids, and target specimen released from a genital swab, oropharyngeal swab, nasopharyngeal swab, buccal swab and rectal swab. The identifying mark can be placed in a patient label area of the cartridge. Inserting the cartridge into the instrument can include inserting the cartridge containing the sample into a vertically oriented loading slot of the instrument. Loading the sample into the cartridge can include flowing a liquid sample into the sample port. The cartridge can be horizontally oriented. The method can further include canceling a testing protocol based on the image of the identifying mark on the instrument graphical user interface. The instrument graphical user interface can be a touchscreen and canceling the testing protocol can include interacting with a portion of the touchscreen.

In general, in one embodiment, a method of operating an instrument for testing a sample suspected of containing a target pathogen includes: (1) receiving a cartridge containing the sample into an opening of the instrument configured to produce a result that contains an indication of a presence, an absence or a quantity of the target pathogen in the sample suspected of containing the target pathogen; (2) capturing an image of an identifiable mark on a cartridge identification label and an indication of the type of testing to be performed in the cartridge; (3) automatically ejecting the cartridge from the instrument if a sample verification test fails; (4) automatically ejecting the cartridge from the instrument if a cartridge verification test fails; (5) automatically ejecting the cartridge from the instrument if a cartridge-instrument interface verification test fails; (6) automatically displaying on a graphical user interface the image of the identifiable mark on the cartridge and a text indicator of the type of testing to be performed in the cartridge.

This and other embodiments can include one or more of the following features. The opening can be a vertically oriented loading slot. The instrument can be configured to maintain the cartridge in a vertical orientation during testing in the cartridge. Capturing an indication of the type of testing to be performed on the cartridge can include parsing a machine-readable barcode. Capturing the image of the identifiable mark can occur within the instrument. The method can further include permitting a user to manually halt testing within a set time period of displaying the image of the identifiable mark on the graphical user interface. The set time period can be ten seconds. The method can further include ejecting the cartridge responsive to receiving a termination command from the user within the set time period. The method can further include initiating a diagnostic assay protocol on the cartridge after elapse of the set time period without receiving a termination command from the user. The method can further include initiating a diagnostic assay protocol on the cartridge to generate a test result, and automatically displaying on the graphical user interface the result that contains an indication of a presence, an absence or a quantity of the target pathogen in the sample suspected of containing the target pathogen.

In general, in one embodiment, a method of operating an instrument for testing a sample suspected of containing a target pathogen includes: (1) loading the sample suspected of containing the target pathogen into a sample port of a cartridge while the cartridge is in a first orientation; (2) adding an identifying mark to a patient label section of the cartridge; (3) orienting the cartridge into a second orientation, wherein the second orientation is orthogonal to the first orientation, and inserting the cartridge into an instrument having a loading slot in the second orientation; and (4) manually advancing the cartridge into the loading slot to secure the cartridge within the instrument, wherein upon securing the cartridge, the instrument automatically initiates a test method including: (1) confirming a quantity of the sample in a loading chamber of the cartridge without any user interaction with the instrument; (2) confirming a position of a component of the cartridge that indicates that the cartridge is ready for use without any user interaction with the instrument; (3) completing a test of the pneumatic integrity of the cartridge without any user interaction with the instrument; (4) displaying the identifying mark on a graphical user interface of the instrument before, during or after successfully completing each of the confirming a quantity of the sample in the loading chamber step, the confirming a position of a component of the cartridge step and the completing a test of the pneumatic integrity of the cartridge step and thereafter initiating a nucleic acid amplification reaction within two or more amplification wells of the cartridge to produce a result that contains an indication of a presence, an absence or a quantity of the target pathogen in the sample suspected of containing the target pathogen; (5) displaying the result on the graphical user interface of the instrument that contains an indication of a presence, an absence or a quantity of the target pathogen in the sample suspected of containing the target pathogen without any user interaction with the instrument; and (6) observing the result on the graphical user interface and interacting with the graphical user interface if the results displayed indicate a quantity or a presence of the target pathogen.

This and other embodiments can include one or more of the following features. The first orientation can be horizontal and the second orientation can be vertical. In the first orientation a cartridge height axis of the cartridge can be normal to a work surface supporting the cartridge during the loading step or the adding step or a work surface supporting the instrument. In the second orientation a cartridge height axis can be parallel to a work surface supporting the cartridge during the loading step or the adding step or a work surface supporting the instrument. In the second orientation a cartridge length axis can be parallel to the work surface supporting the cartridge during the loading step or the adding step or a work surface supporting the instrument. In the second orientation a cartridge length axis of the cartridge can be normal to a rear wall of the instrument. In the second orientation a cartridge width axis of the cartridge can be normal to a base of the instrument. The method can further include moving the cartridge into the second orientation by rotating the cartridge about a cartridge length axis.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an isometric view of an exemplary instrument configured to be used with a number of various CLIA waived point of care testing methods.

FIG. 2 is a top view of an exemplary integrated diagnostic cartridge configured to be used with an instrument as shown in FIG. 1

FIG. 3 is a schematic layout of an integrated diagnostic cartridge configured to be used with an instrument according to an embodiment described herein.

FIGS. 4A and 4B illustrate two exemplary techniques of applying an identifying mark to the label portion of an integrated diagnostic cartridge.

FIG. 5 shows a user loading a sample into an integrated diagnostic cartridge using a sample loader, such as a bulb, syringe or pipette.

FIG. 6 illustrates the integrated diagnostic cartridge after sample loading is completed and the user seals the sample port assembly by closing a cap.

FIG. 7 illustrates a perspective view of a diagnostic instrument and an integrated diagnostic cartridge supported by a tabletop when the integrated diagnostic cartridge is in a first orientation.

FIG. 8 illustrates a perspective view of a diagnostic instrument and an integrated diagnostic cartridge during an exemplary cartridge handling movement for inserting the cartridge into the instrument in a second orientation.

FIG. 9 illustrates an instrument with an integrated diagnostic cartridge contained substantially therein while running a diagnostic testing protocol.

FIG. 10 is a view of an exemplary security screen on an instrument GUI.

FIG. 11 is a view of an exemplary ready screen on an instrument GUI.

FIG. 12 illustrates a properly oriented cartridge being inserted into the instrument.

FIG. 13 illustrates an exemplary new cartridge screen on an instrument GUI for confirming the cartridge test, sample type and patient identifying mark.

FIG. 14 illustrates an exemplary reading cartridge screen on an instrument GUI for observing while the instrument performs a number of verification tests.

FIG. 15 illustrates an exemplary cartridge verification screen on an instrument GUI for observing that instrument has completed the verification checks and the testing protocol has started.

FIG. 16 illustrates an exemplary running test screen on an instrument GUI for observing the progress of a currently conducted testing protocol.

FIG. 17 illustrates an exemplary last completed test screen on an instrument GUI for observing the results of a testing protocol for the patient identified in FIG. 16.

FIG. 18 illustrates an exemplary instrument ejecting an integrated diagnostic cartridge.

FIG. 19A-B illustrates exemplary test result summary screens on an instrument GUI for observing results from previously conducted testing protocols.

FIG. 20 illustrates an exemplary quarantine screen on an instrument GUI.

FIG. 21A illustrates an exemplary positive control screen on an instrument GUI for observing a result of a successful positive control test.

FIG. 21B illustrates an exemplary negative control screen on an instrument GUI for observing a result of a successful negative control test.

FIG. 21C illustrates an exemplary failed control screen on an instrument GUI for observing a result of an unsuccessful negative control test.

FIG. 22 illustrates is an exemplary error screen on an instrument GUI for displaying an instrument fault. Specifically, the instrument fault or error was a mechanical interface issue between the instrument and the cartridge.

FIG. 23 illustrates an additional exemplary error screen on an instrument GUI for displaying a cartridge fault. Specifically, the instrument determined that the cartridge was expired and may not be used for sample testing.

FIG. 24 is a perspective exploded view of components within the diagnostic instrument of FIG. 1.

FIG. 25 is an enlarged perspective view of an exemplary clamping subsystem of FIG. 24 showing an integrated diagnostic cartridge disposed between a fixed bracket assembly and a moving bracket assembly.

FIG. 26A is an enlarged perspective view of a portion of a loading assembly for accepting and ejecting an integrated diagnostic cartridge as shown in various views of FIGS. 24 and 25. An integrated diagnostic cartridge is shown in position when loaded into the integrated diagnostic instrument.

FIG. 26B is an enlarged view of a distal most portion of the loading assembly of FIG. 26B.

FIGS. 27A-B are enlarged perspective views of an upper portion of an integrated diagnostic cartridge prior to and engaged with, respectively, a cartridge alignment feature of an exemplary loading assembly of the various views of FIGS. 24, 25 and 26A.

FIGS. 28A-B are enlarged perspective views of a lower portion of an integrated diagnostic cartridge prior to and engaged with, respectively, a cartridge alignment feature of an exemplary loading assembly of the various views of FIGS. 24, 25 and 26A.

FIGS. 29A and 29B are perspective and vertical plane views, respectively, of the patient label imaging assembly of FIG. 24. FIG. 29A includes a perspective view of an integrated diagnostic cartridge in an exemplary loaded position where the patient label, loading port cap and sample levels are within the various optical fields of the imaging assembly. FIG. 29B includes a section view of the cartridge of FIG. 29A.

FIG. 30 is schematic view of a representative computer control system for an exemplary point of care diagnostic instrument.

FIG. 31 is a flow chart of an exemplary method of a workflow for operating a point of care diagnostic instrument for testing a sample suspected of containing a target pathogen.

FIG. 32 is a flow chart of an exemplary method of a workflow for operating a point of care diagnostic instrument for testing a sample suspected of containing a target pathogen.

FIG. 33 is a flow chart of an exemplary method of a workflow for operating a point of care diagnostic instrument for testing a liquid sample suspected of containing a pathogen.

FIG. 34 is a flow chart of an exemplary method of a workflow for operating a point of care diagnostic instrument for testing a sample suspected of containing a pathogen.

FIG. 35 is a flow chart of an exemplary method of a workflow for operating a point of care diagnostic instrument for testing a sample suspected of containing a pathogen.

FIG. 36 is a flow chart of an exemplary method of a workflow for operating a point of care diagnostic instrument for screening an individual desiring access to a location, an event or an activity.

### DETAILED DESCRIPTION

### 1 Method of Using a Point of Care Instrument—User Experience

### 1.1 Introduction

### 1.1.1 Impediments to Expanded Use of Point of Care Testing Systems

Commercially available point of care systems pose challenges to health care personnel when conducting molecular diagnostic tests. Numerous handling steps related to sample custody and security protocols further complicate the user workflow when using a point of care system to diagnose a patient of a suspected infectious disease. Each additional handling step prior to initiating an instrument testing protocol introduces the potential for error and could lead to a false test result. Falsely detecting an infectious disease has tremendous consequences, such as allowing the disease to further progress and/or transmitting the disease to a new host. Thus, a need exists for improved easy-to-use point of care diagnostic systems for reliably conducting molecular diagnostic testing in the point of care environment.

A CLIA waived laboratory test is characterized as simple laboratory examinations and procedures that have an insignificant risk of an erroneous result. Moreover, user compliance with a CLIA waiver certification requires persons performing the CLIA waived test follow all of the manufacturer's instructions related to intended use as well as all limitations of the waived testing procedure. A manufacturer's intended use, limitations and instructions will vary depending upon the configuration of the diagnostic instrument and integrated cartridge design and workflow. Additional requirements related to intended use and limitations on testing include, by way of example and not limitation, (i) observing storage and handling requirements for test system and components; (ii) adhering to the expiration date of the testing system and reagents as applicable; (iii) performing quality control as required by the manufacturer; and (iv) reporting patients' test results in the units described by the manufacture's instruction or package insert.

In addition, guidance provided by U.S. Department of Health and Human Services (HHS) and the Centers for Medicare and Medicaid Services (CMS) highlights a number of additional laboratory practice recommendations to ensure accuracy and reliability of waived testing. By way of example and not limitation, the guidance recommends (a) appropriate sample collection; (b) appropriate sample storage and labelling; (c) understanding, knowledge and compliance with manufacturer's instructions for each test performed; (d) understanding and knowledge of how to communicate test results; (e) understanding and knowledge of how to identify inaccurate results or failures of a testing system or integrated cartridge; (f) positive identification of patient and specimen; (g) compliance with sample handling, preservation and custody compliance. Additional recommended actions and guidance for CLIA waived testing procedures are available from https://www.cdc.gov/hiv/testing/nonclinical/clia.html; https://www.cms.gov/Regulations-and-Guidance/Legislation/CLIA/index?redirect=/clia; https://www.cms.gov/Regulations-and-Guidance/Legislation/CLIA/CLIA Brochures; and “CLIA Brochure—How to Obtain a CLIA Certificate of Waiver” (available online as of at least March 2016 and accessed Jul. 9, 2020).

While interest continues in more rapid near patient and point of care test results and expanded use of CLIA waived testing, the above recommendations, guidelines and requirements make clear that obstacles remain to widespread adoption. The various workflows described herein provide an added benefit of assisting in the compliance to one or more of the above identified recommendations, guidelines and requirements. As will be clear in the description of the embodiments that follow, the improved workflows are intended to seamlessly couple straightforward sample collection and identification procedures that are readily adaptable to user preference with easy to follow instructions provided by the instrument with minimal user interaction and a high degree of automated functionality.

Still further, embodiments of the workflows described below immediately alert the instrument operator if a fault is identified that will impair or lead to an invalid test procedure. In general, these faults are grouped as relating to a sample, a cartridge, any interface between a cartridge and instrument or in the instrument itself. Of particular interest for operations in the near patient and point-of-care environments, determination of potential faults “immediately” includes providing an alert of potential fault in the first few minutes (i.e., less than 180 seconds), in less than a minute (i.e., less than 60 seconds) or less than 30 seconds from the introduction of an integrated point of care cartridge into a diagnostic instrument. In contrast, conventional near patient and point of care workflows do not provide early automatic fault detection, only report faults after many minutes have passed or, worse, with an error message or fault indication at the end of a sample processing cycle. While the exemplary methods described herein may be modified to reflect specific testing according to a particular manufacturer and cartridge, it is believed that immediate, automatic confirmation of: (1) sample adequacy; (2) cartridge integrity and (3) instrument-cartridge operability will provide additional benefits to those performing CLIA waived testing. As a result, implementation and practice of embodiments of the workflow methods described herein saves valuable clinical time by virtue of the automatic confirmation or fault detection steps performed upon initiation of a sample processing sequence.

Disclosed herein are advantageous methods of performing rapid molecular diagnostic testing at the point of care, in near patient environments or when performing CLIA waived laboratory tests. The embodiments described herein will realize simplified and time saving advantages for users and operators of point of care diagnostic systems while also aiding in compliance with one or more of the recommendations, guidelines and requirements above. Importantly and advantageously, the various methods described herein are agnostic to sample type, specific integrated cartridge or point of care diagnostic instrument configuration, form factor or design. Instead, the methods and workflows described herein can be implemented on any of a wide variety of point of care, near patient or CLIA waived system and integrated cartridge types. The various embodiments are disclosed in relation to a specific integrated cartridge and instrument combination solely for the purposes of clarity and understanding. As such, the various exemplary workflows and methods may be used to readily realize the many benefits of CLIA waived laboratory testing protocols by operators with limited training or even untrained operators.

### 1.1.2 Overview of an Exemplary Workflow

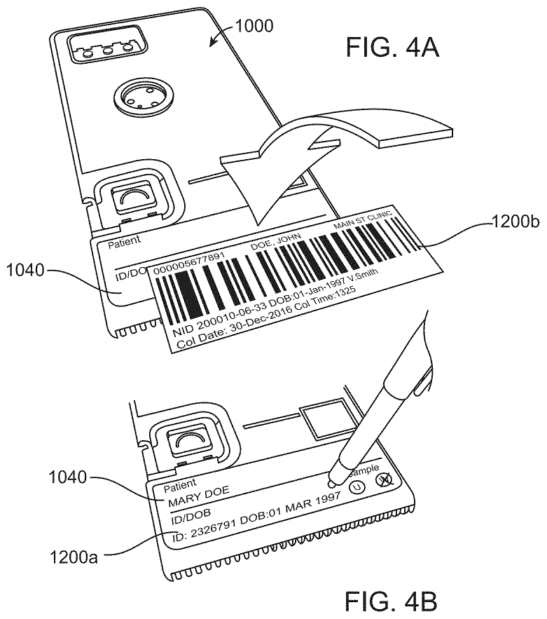

One aspect of the invention provides methods of operating an instrument for testing a sample suspected of containing a target pathogen, comprising (a) loading the sample suspected of containing a target pathogen into a sample port assembly of a cartridge, (b) adding an identifying mark to a patient label area of the cartridge, (c) inserting the cartridge into an opening of the instrument until the cartridge is positioned within the instrument with the identifying mark within a field of view of a label imaging camera, (d) observing on the graphical user interface an indication of the type of test to be performed on the cartridge and an image of the identifying mark on the label area of the cartridge, (e) interacting with the graphical user interface to eject the cartridge if the image of the identifying mark or the indication of the type of test is incorrect, and (f) removing the cartridge from the opening of the instrument after the cartridge is automatically ejected from the opening. FIGS. 4A-18 illustrate the various steps and exemplary graphical user interface (GUI) displays that correspond to the workflow listed above. Following these or similar steps describe one method of operating a diagnostic instrument for testing a sample suspected of containing a target pathogen with a matched integrated diagnostic cartridge.

### 1.1.3 Instrument Introduction

The following methods of operating an instrument for molecular diagnostic testing will be described in context to embodiments of a diagnostic system comprising a diagnostic instrument and a matched integrated diagnostic cartridge. By way of introduction, a diagnostic instrument 2000 will be described according to several subsystems and assemblies. The diagnostic instrument has a plurality of subsystems or assemblies matched for interaction with and testing of a sample contained in the cartridge. Instrument interactions with the cartridge may include, but are not limited to, accepting a cartridge, performing verification tests, executing sample processing and amplification steps, and capturing images of at least one portion of the cartridge.

Such point of care instrument subsystems or assemblies may include any of a wide range of electro-mechanical, magnetic, hydraulic, mechanical, pneumatic, thermal, optical, image processing and display subsystems for performing the diagnostic test based on the specific design parameters of an instrument and cartridge combination. Accordingly, one skilled in the art of integrated cartridge and instrument designs would appreciate and be desirous of designing in and constructing an instrument according to a wide variety of different testing methodologies and cartridge designs to realize the advantages described herein. Still further, those skilled persons would readily realize that modification to one or more cartridge or instrument features would bring the advantages described herein to cartridges and systems of their own making.

In some implementations, a mechanical subsystem is included for orchestrating, under control of the computer system, the various physical interactions between a diagnostic instrument and a matched integrated diagnostic cartridge. In furtherance of simplifying implementation of one or more of the recommendations, guidelines and requirements above, the mechanical subsystem can be configured to accept a cartridge in a preferred orientation, place the cartridge in the preferred orientation, and perform a plurality of instrument and/or cartridge verification tests. Some implementations include a pneumatic subsystem for advancing fluids throughout the cartridge and a thermal subsystem for initiating and maintaining an amplification reaction. Additionally, the image capture system employed for the image capture of the patient label may not be the only imaging system of an instrument. Some instrument configuration may include an optical subsystem for capturing images of one or more portions of a cartridge. Other implementations include an optical subsystem for illuminating and capturing images of one or more portions of a cartridge.

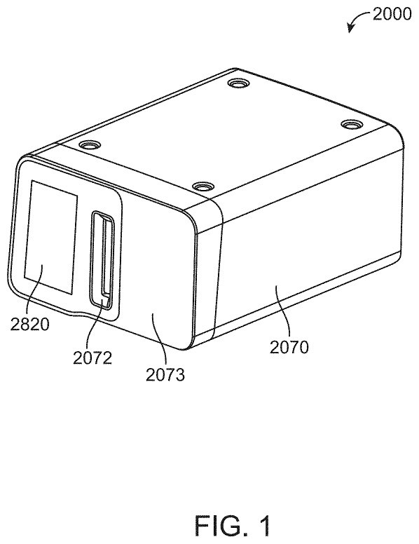

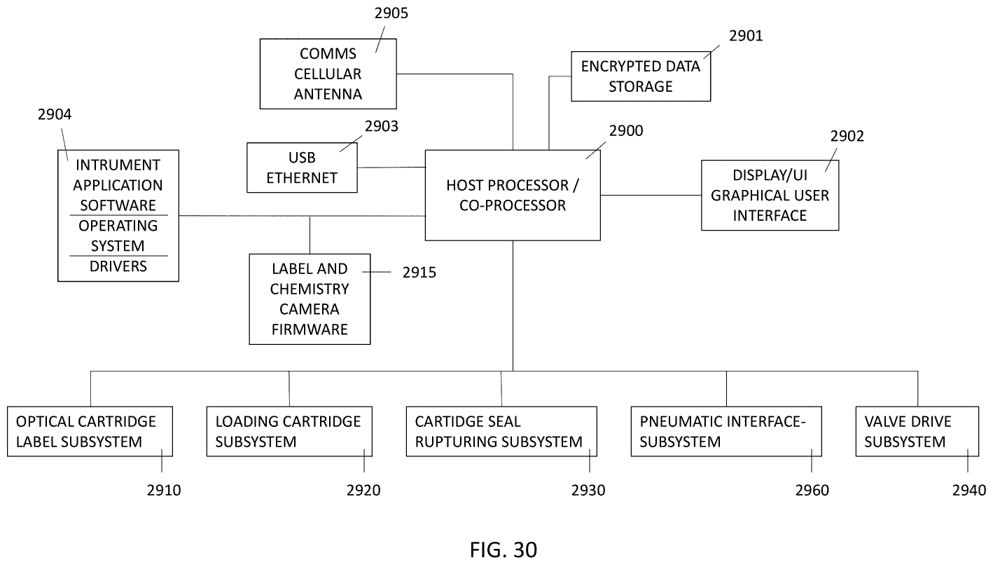

An exemplary diagnostic instrument 2000 is shown in FIG. 1. This view of the instrument front 2073 shows a graphical user interface (GUI) 2820 adjacent to an opening 2072 to receive an integrated cartridge matched to the instrument 2000. Advantageously, GUI 2820 enables easy user or operator communications and interaction with the instrument. As will be made clear in the examples which follow, the displays presented on the GUI further compliance or implementation of a number of recommendations, guidelines and requirements detailed above. For example, a GUI display provides easy to read as well as easy to comprehend any instructions relating to an interaction with or an operation of the instrument. Regardless of the specific components or subsystems implemented in a specific instrument design, such components and subsystems as well as the GUI are under the control of an appropriately configured instrument computer control system. The appropriately configured instrument computer control system includes instructions in computer readable code for coordinating the synchronous performance of the one or more of the operations described herein related to receiving, handling, processing and analyzing a suspected sample in a cartridge. Advantageously, an appropriately configured instrument computer control system includes a number of instructions which when implemented execute any of a number of automatically performed processes which may be accomplished without direct operator or user interaction. Examples of such automatically performed processes include, for example, performance of steps to ensure a sample is adequate, properly loaded, an identifying mark is present on a patient label, one or more cartridge integrity checks or one or more instrument integrity checks or one or more instrument-cartridge integrity checks. An exemplary instrument computer system is further detailed below with regard to FIG. 30.

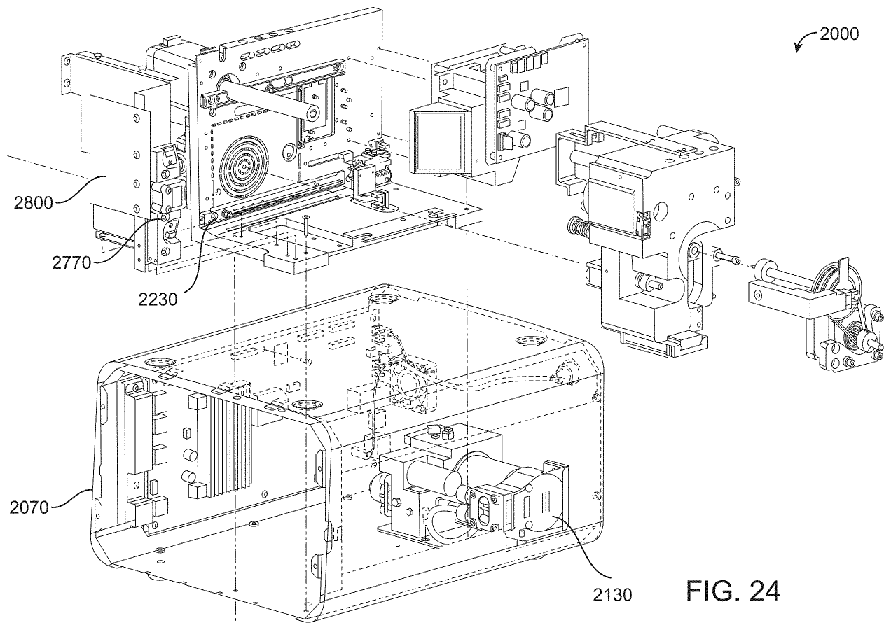

In one specific illustrative example to further understanding of the inventive workflows, the various workflow embodiments will be described as implemented in the point of care diagnostic system 2000. Various components and subsystems of the system 2000 are illustrated and described further below with regard to the various views of FIGS. 24, 25, 26A, 26B, 27A, 27B, 28A, 28B, 29A and 29B. Additional details of the components and operations of the instrument 2000 are further described in U.S. Non-Provisional patent application Ser. No. 16/655,007, entitled “Diagnostic System” filed Oct. 16, 2019 and U.S. Non-Provisional patent application Ser. No. 16/655,028, entitled “Diagnostic System” filed Oct. 16, 2019, each of which is incorporated herein by reference for all purposes.

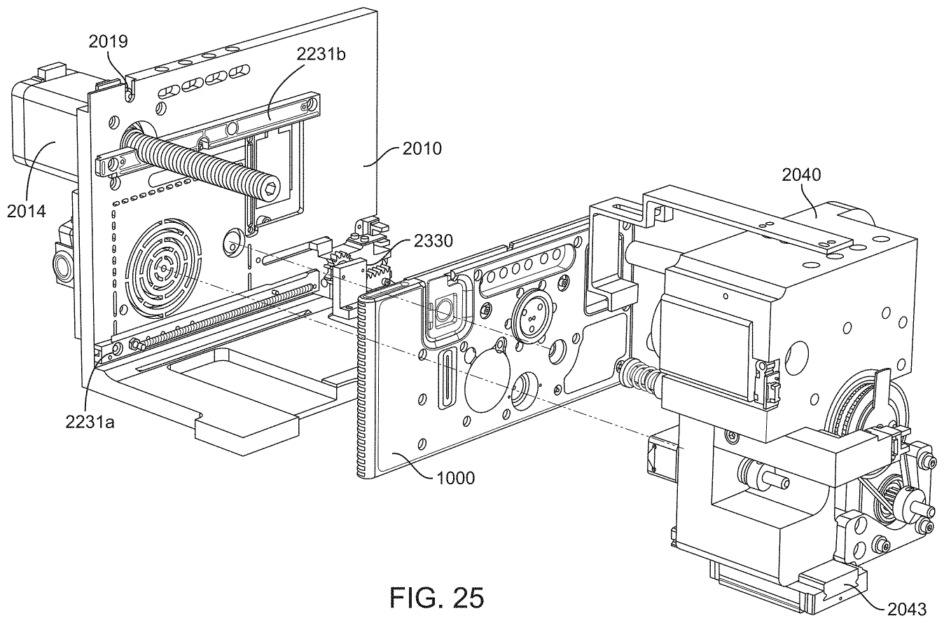

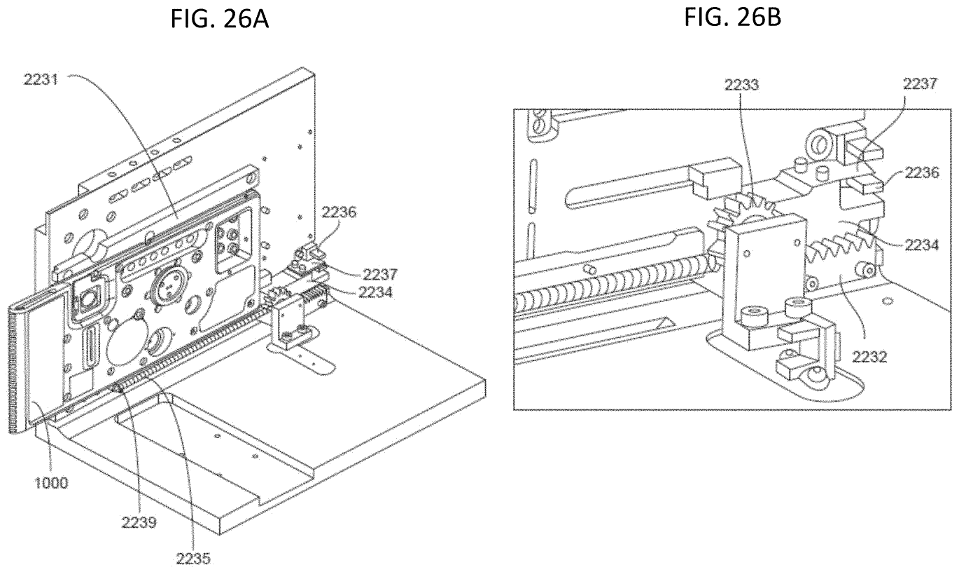

By way of a brief introduction, FIG. 24 is a perspective exploded view of components within the diagnostic instrument of FIG. 1. FIG. 25 is an enlarged perspective view of an exemplary clamping subsystem of FIG. 24 showing an integrated diagnostic cartridge disposed between a fixed bracket assembly and a moving bracket assembly. FIG. 26A is an enlarged perspective view of a portion of a loading assembly for accepting and ejecting an integrated diagnostic cartridge as shown in various views of FIGS. 24 and 25. An integrated diagnostic cartridge is shown in position when loaded into the integrated diagnostic instrument. FIG. 26B is an enlarged view of a distal most portion of the loading assembly of FIG. 26B.

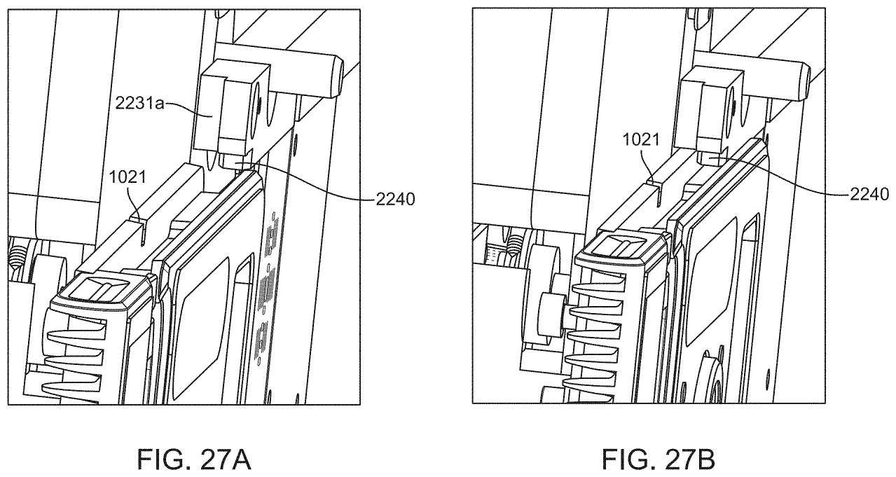

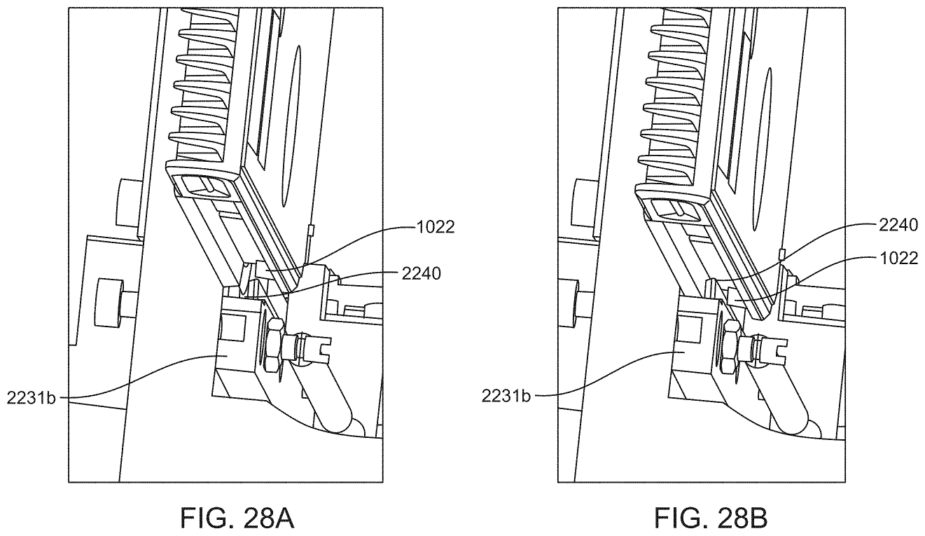

FIGS. 27A-B are enlarged perspective views of an upper portion of an integrated diagnostic cartridge prior to and engaged with, respectively, a cartridge alignment feature of an exemplary loading assembly of the various views of FIGS. 24, 25 and 26A. FIGS. 28A-B are enlarged perspective views of a lower portion of an integrated diagnostic cartridge prior to and engaged with, respectively, a cartridge alignment feature of an exemplary loading assembly of the various views of FIGS. 24, 25 and 26A.

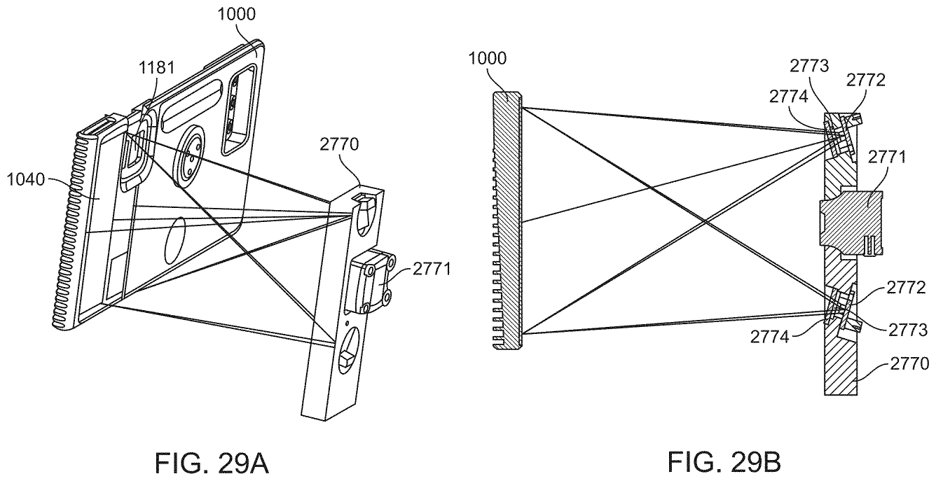

FIGS. 29A and 29B are perspective and vertical plane views, respectively, of the patient label imaging assembly of FIG. 24. FIG. 29A includes a perspective view of an integrated diagnostic cartridge in an exemplary loaded condition where the patient label, loading port cap and sample levels are within the various optical fields of the imaging assembly. FIG. 29B includes a section view of the cartridge of FIG. 29A showing the respective lighting provided to both the label area as well as the sample. Patient label imaging assembly 2770 is configured to illuminate and capture images of the patient label and loading module. As shown in FIG. 24, the label imaging assembly is mounted to the antenna ground plate 2800 and comprises a camera 2771, LED 2772, aperture 2773 and diffuser 2774, shown in FIGS. 29A and 29B. The label imaging assembly 2770 will include at least one, but preferably more than one (e.g., two or three), LEDs 2772 for illuminating the patient label area 1040 and the loading module while minimizing shadows cast in the patient label area. The aperture 2773 defines an opening to transmit and reshape illumination by LEDs to reduce off axis light and stray light from affecting the patient label image quality.

In an implementation consistent with the workflow embodiments described herein, the label imaging assembly 2770 may also be configured to automatically image the sample port assembly to verify adequate sample is loaded into a cartridge as part of a sample verification procedure prior to running a diagnostic test. In achieving additional time savings, it is advantageous to determine a sufficient sample volume is present in the loading module as an initial verification step. Cartridges with insufficient sample volume may be automatically ejected during initial sample verification or cartridge verification processes described herein.

### 1.1.4 Cartridge Introduction

The embodiments described herein relate to a disposable single use device (a “cartridge”) used in methods of operating an instrument for testing a sample suspected of containing a target pathogen. It is to be appreciated that the following embodiments and configurations are used solely for the purpose of describing in detail by way of illustration and example for the purposes of clarity and understanding. It is readily apparent to those skilled in the art in light of the teachings of these embodiments that certain changes and modifications can be made thereto without departing from the spirit or scope of the appended claims.

In some embodiments, the cartridge contains a plurality of modules for performing a variety of functions in order to affect the diagnostic test including, but not limited to, a loading module, a lysis module, a purification module, and an amplification module. In some implementations, the loading module is configured to receive a sample, minimize the spilling of the sample, and prepare the sample for lysis. In another implementation, a cartridge includes an appropriate lysis module for disrupting cells walls/cell membranes and releasing inter-cellular materials, e.g. nucleic acids. In another implementation, a cartridge contains a purification module for isolating and/or enriching nucleic acid from a lysed sample. In another implementation, a cartridge includes an amplification module for generating and/or detecting a signal from target amplicon, indicative of the presence of target pathogen in the sample. Additionally, the cartridge may be configured to store all liquid and dried reagents on-board to perform an assay, such that a user or operator is only required to load a patient sample into the cartridge prior to inserting into the instrument.

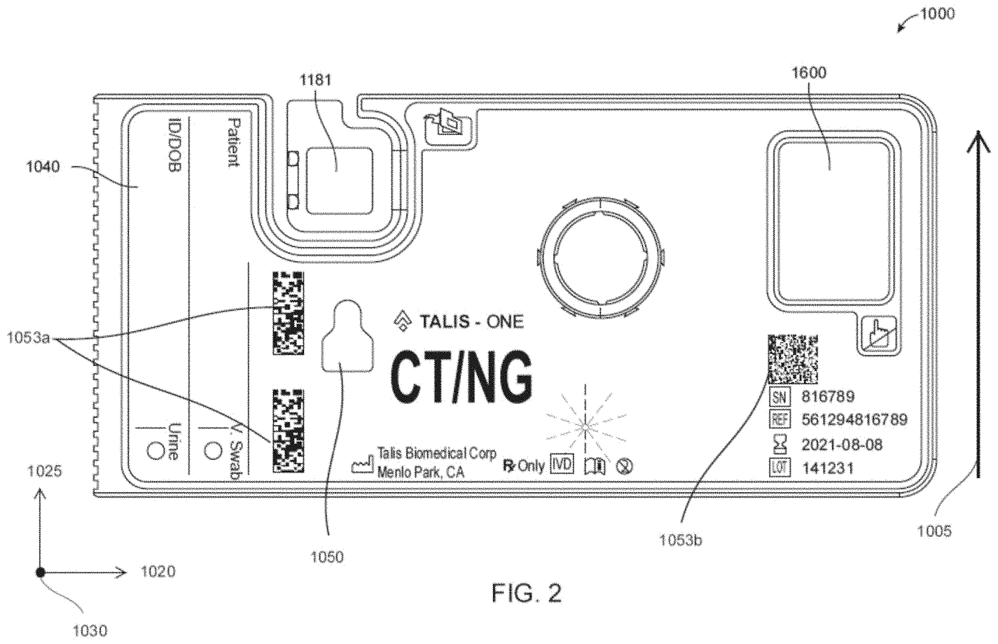

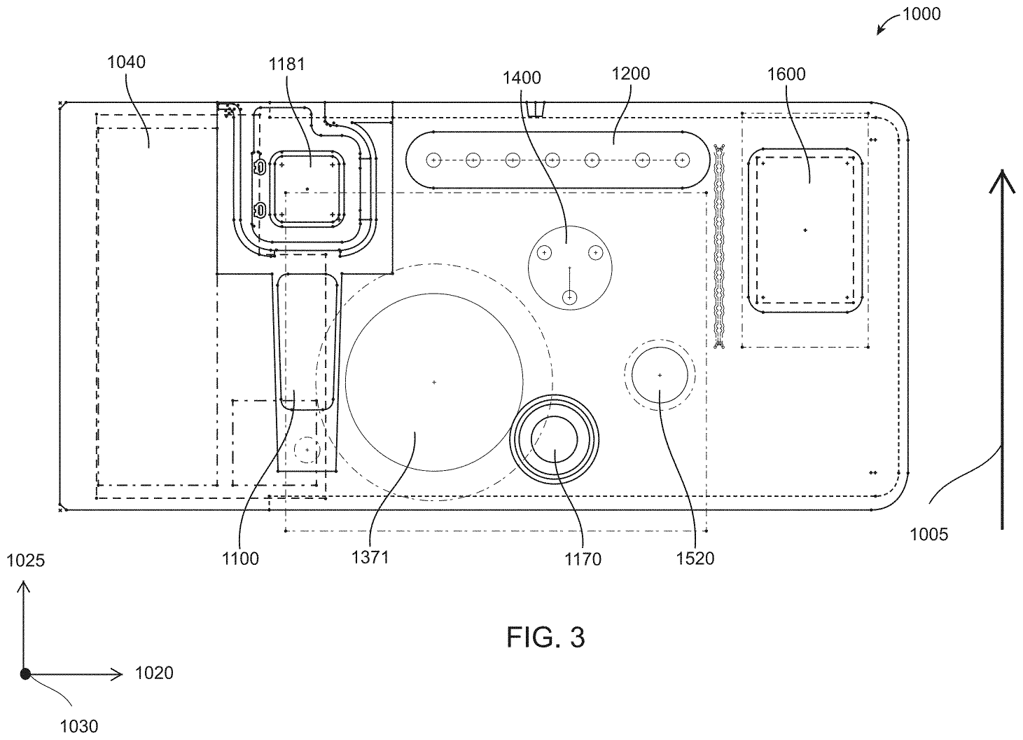

FIG. 2 and FIG. 3 are a top down views of an exemplary integrated diagnostic cartridge 1000. In these illustrative embodiments, the cartridge 1000 includes a length, width, and height and includes assemblies enabling the loading, lysing, purification, and amplification of a suspected target pathogen within a biological sample. In the illustrated embodiment, a loading module is on one end of the cartridge adjacent to a patient label area 1040, while an amplification module containing a reaction area 1600 is on the other end of the cartridge. Further to the compact and modular design aspects of the various cartridge embodiments, a lysis module and purification module are arranged to occupy the portion of the cartridge existing between the loading module and the amplification module. As such, the diagnostic cartridge may comprise additional features for supporting the purification and amplification of suspected target nucleic acids contained in a patient sample. Further, the placement of such modules may also take advantage of a vertical orientation within the instrument as described further herein. Details regarding the exemplary cartridge modules can be found further below.

In certain implementations, a cartridge comprises a plurality of fluidic channels, ducts, and pathways formed within the cartridge to form a fluidic network for transporting a sample and various substances to different modules of a cartridge. In another implementation, one or more channels in the fluidic network are configured to accommodate pressurized air for motivating fluids to cartridge modules. In other implementations, one or more channels are purposed for venting and rerouting air or gas within the cartridge when a sample is loaded. Additionally, the fluidic network may further comprise a plurality of vias, e.g. openings, passages, or ports configured for passing fluids there through from a first plane of the cartridge to a second plane of the cartridge.

### 1.2 Loading Sample

### 1.2.1 Overview of Loading Step

In one aspect of the invention, a user or operator begins the method of operating an instrument for testing a sample suspected of containing a target pathogen by loading a biological sample collected from a patient into an integrated diagnostic cartridge. The term “loading” refers to the process of transferring a collected patient sample from a sample collection container and into an opening integrally formed within the cartridge. In a further embodiment, loading further includes closing or sealing the opening of the cartridge to prevent spillage of the sample. In some embodiments, the sample is transferred from the sample collection container to the cartridge using a sample transfer device. In a further embodiment, a sample transfer device is a sample loader, such as a bulb, pipette, syringe, or any other device useful for loading a sample into a cartridge. In some implementations, the diagnostic system can include a plurality of sample loaders to be used for the loading step. In other implementations, each individual cartridge can be packaged with a sample transfer device, such as a syringe, bulb, swab, scraper, biopsy punch, or other tool for a user to collect a sample.

### 1.2.2 Biological Samples

A patient specific biological sample may be collected by the patient, a physician or other health care worker. The collection of a patient biological sample type is based on the diagnostic assay protocol to be conducted using a diagnostic system. In general, liquid biological samples are commonly collected by draw or liquid capture into a sample collection container. A liquid sample as used herein refers to biological fluids collected from a patient. Additionally, biological samples may be collected with swabs through self-collection or collected by a health care worker. Accordingly, a liquid sample also includes a liquid suspension comprising a transport and/or storage liquid media containing cells, pathogens, or other target specimen released from a swab collected from a patient. Exemplary liquid samples include urine, blood, sputum, saliva, or other oral fluids. Exemplary swab types used to produce a patient specific liquid sample include a vaginal swab, an oropharyngeal swab, a nasopharyngeal swab, a buccal swab, a genital swab, a rectal swab, a wound swab, or a dermal swab. In some embodiments, the liquid sample is urine, blood, sputum, saliva, oral fluids, or a suspension released from a genital swab, oropharyngeal swab, nasopharyngeal swab, buccal swab, or rectal swab. In a preferred embodiment, the liquid sample is urine, blood, sputum, saliva, or oral fluids.

Liquid sample volumes loaded for any particular test will vary based on a number of factors, such as diagnostic assay protocol, cartridge operations, e.g. a mode of fluid movement within a cartridge, and cartridge characteristics, e.g. volume. In various alternatives of the method, the liquid sample has a volume between 0.2 ml and 5 ml, inclusive, the volume of the liquid sample is between 0.5 ml and 1.5 ml, inclusive or the volume of the liquid sample is approximately 1 ml.

### 1.2.3 Loading Module

### 1.2.3.1 Sample Port Assembly

In many embodiments, a diagnostic cartridge contains a loading module comprising at least one of a sample loading port, a sample input well, a fill chamber, or any other opening for providing the user limited access to the interior of the cartridge for loading a patient sample. In one implementation, the loading module is a sample port assembly 1100. In addition to the sample port, the sample port assembly may further comprise a loading chamber for storing the patient sample until said sample is advanced to other locations within the cartridge for sample processing. Alternatively, the sample can be loaded via a puncturable septa or large one-way valve. In certain implementations, the sample port assembly contains a cap 1181 configured to be opened to permit addition of a sample through the opening and configured to be closed to seal the opening, thus preventing any liquids from escaping the cartridge. Preferably, the cap is configured to prevent the re-opening after a sample is added and said lid is closed. In another implementation, the opening is preferably is air-tight when sealed by the cap for embodiments where pressurization is used to advance fluids to the plurality of modules.

FIGS. 2 and 3 are a top view of the representative point of care testing cartridge 1000 designed to be used with the exemplary diagnostic instrument 2000 described herein. The loading module of the exemplary cartridge comprises a sample port assembly 1100 for loading a sample. In such embodiments, the sample port assembly 1100 comprises an opening, e.g. sample port 1140 (not shown), for introducing a patient sample into a cartridge. The sample port assembly further includes a cap 1181 for sealing and preventing any liquids from escaping outside the cartridge after the sample is loaded. In these views, the sample port assembly 1100 is shown with the cap 1181 closed. Additionally, shown within the exemplary sample port assembly is a patient label area 1040 located at one end of the cartridge.

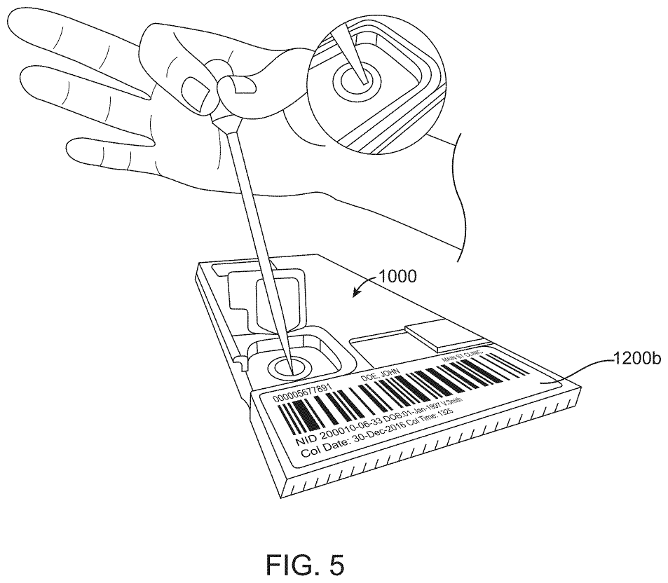

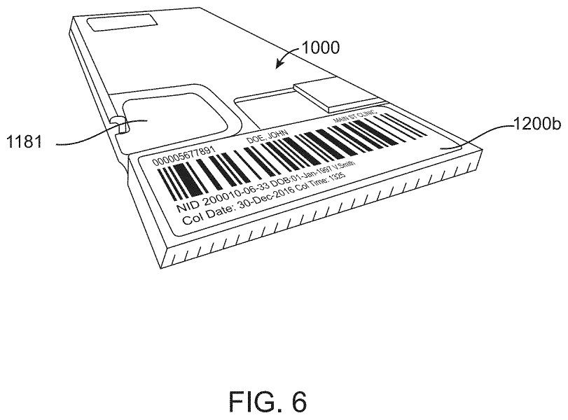

In some implementations, after the collection of a patient sample in a sample collection container, the user or operator loads the patient sample suspected of containing a target pathogen into a sample port assembly using a sample transfer device, as shown in FIG. 5. In a further implementation, the loading step further includes sealing a cartridge sample port after transferring the sample. FIG. 6 illustrates the cartridge once the cap 1181 closed. Additionally, the closure on the cartridge sample port may be irreversible or tamper proof so that once closed or sealed a user may not readily access the sample port.

### 1.3 Adding Identifying Mark

### 1.3.1 Overview of Marking Step

In another aspect of the invention, in addition to the successful loading of the sample, the method of comprises adding an identifying mark 1200 to a patient label area 1040 of the cartridge. In a variety of implementations, identifying marks are used for supplying the user and/or instrument computer system with patient, sample, and/or testing information. In many implementations, the identifying mark is placed in a patient label area of the cartridge. Such patient label area is configured to accommodate such a mark applied by an operator. The patient label area will be positioned appropriately within the instrument to allow an image to be captured of the patient label area 1040 containing the identifying mark 1200. As discussed below, an operator may employ any of a wide range of marks suited to any clinical workflow. However, as part of ensuring that patient and sample integrity is maintained, if no identify mark is detected by the instrument, the cartridge will be ejected. The GUI will display an appropriate message for the operator to place an identifying mark on the patient label area of the cartridge and re-insert the cartridge into the instrument.

### 1.3.2 Identifying Marks

Cartridge marking and identification refers generally to the steps performed by an operator for adding an identifying mark to the cartridge to identify patient and other important information associated with a particular test run on the diagnostic instrument. In many embodiments, the identifying mark identifies a patient from which the sample is acquired. In further embodiments, such information provided by the identifying mark may identify the patient by name, ID number, and/or date-of-birth.

In additional embodiments, the identifying mark further can be a marking for indicating the sample type. As described herein, the sample type can be selected from the group consisting of urine, blood, saliva, sputum, oral fluids, and target specimen released from genital swabs, oropharyngeal swabs, nasopharyngeal swabs, buccal swabs, and rectal swabs. In some implementations, adding an identifying mark step comprises marking a pre-printed box, circle, or any other equivalent geometric shape or area in the patient label area of the cartridge indicating the sample type contained therein. As shown in FIG. 4B, the user marked a circle in the patient label area to indicate the sample type. The view of FIG. 2 also indicates a pre-printed area where sample type may be indicated by simply marking the label appropriately.

### 1.3.2.1 Handwritten Labels

In various implementations, all or a portion of the identifying mark is handwritten 1200a by the operator. Particularly, in some implementations, the identifying mark comprises handwriting information in the patient label area for identifying the sample. In additional embodiments, a patient label area is configured to permit a user to write directly onto the cartridge with a pen or marker. A blank patient label area before the addition of the identifying mark is shown in FIG. 2. An example of a handwritten identifying mark in the patient label area is shown in FIG. 4B. A wide range of information may be added to the patient label area in order to identify the patient and sample. Examples include writing in the patient's name, ID, date-of-birth, sample type, or any other entry to associate a given test result with the provided information used to make the identifying mark. FIG. 4B illustrates how a user may write the indicated information and mark the pre-printed circle to indicate the sample type. In an alternative embodiment, the identifying mark may simply be any marking preferred by the operator to denominate patient samples.

### 1.3.2.2 Printed Labels

In an alternative implementation, all or a portion of the identifying mark is a machine readable code 1200b with embedded information. In such implementations, one or more machine readable codes are embedded with information to identify a patient or sample, e.g. a barcode embedded with a patient ID number, patient name, clinic name, patient DOB, sample collection date, and sample collection time. FIG. 4A illustrates an exemplary barcode 1200b with patient identification information sized for application to the patient label area. The machine readable identifying mark can be printed on a label wherein adding or placing the identifying mark on the cartridge comprises affixing the printed label in the patient label area to identify the sample. Examples of machine readable codes are barcodes, QR codes, or any other suitable machine readable markings embedded with information. In a preferred implementation, the identifying mark is a barcode. Additionally or optionally, the identifying mark in a patient label area may include both machine readable and printed or handwritten markings.

### 1.3.2.3 Test Type and Cartridge Information

In another implementation, one or more machine readable codes differing from those used for patient identification information is embedded with information to identify test type and/or communicate cartridge manufacturing details. In some embodiments, the positioning of the one or more machine readable codes is adjacent to the patient label area 1040. In other implementations, the positioning of the one or more machine readable codes is located anywhere within a field of view of an optical subsystem. The convenient positioning of the machine readable codes further permits the simultaneous processing of information embedded within the machine readable code by an instrument computer system when an image of the patient label area is captured. Such embedded information in barcode 1053a is used to initiate an appropriate testing protocol or sequence of instructions conducted by the instrument to perform the correct diagnostic test. In such embodiment, the embedded test type may further instruct the instrument to perform at least one verification test before executing the appropriate testing protocol or sequence. Additionally, the one or more machine readable codes, e.g. supply the instrument and/or user with information relating to cartridge manufacturing. In some embodiments, it may be beneficial to provide duplicated machine readable codes for reliably providing the instrument with information, e.g. testing or manufacturing information, before initiating the testing protocol. In such embodiment, if one of the machine readable codes is defective, a second machine readable code can be read by the instrument. The exemplary cartridge in FIG. 2 has a duplicated machine readable code 1053a adjacent to the patient label area for providing such information. Additionally, in this exemplary embodiment, the machine readable code 1053b encodes the cartridge Unique Device Identifier (UDI) and may also contain cartridge specific calibrations and instrument run settings dependent on the type of diagnostic assay. In a further optional embodiment, a select amount of information embedded within the machine readable code can be included on the label in human-readable format. In the exemplary cartridge shown in FIG. 2, the operator can read the limited amount of manufacturing information available on the cartridge, e.g. serial number, manufacturing code, expiration date, and lot number.

### 1.3.3 Patient Label Area Location

As described herein, patient information relating to the diagnostic test may be provided by the operator by either handwriting the information in the patient label area or adding a printed label with a machine readable code to the patient label area. In some implementations, the patient label area 1040 is a component of the loading module and is adjacent to the sample port assembly 1100. This embodiment is shown in the exemplary cartridge of FIGS. 2-6. In some embodiments, the patient label area is located in a position corresponding to a field of view of an instrument optical subsystem configured for capturing an image of the patient label area. Description of the instrument optical systems enabling the image capture and analysis are further described herein.

### 1.3.4 Order of Loading/Marking

According to various embodiments, loading the sample into a sample port assembly 1100 of a cartridge and adding an identifying mark is required prior to inserting the cartridge into the instrument. However, in some implementations, loading the patient sample into a sample port assembly is performed before adding an identifying mark to the cartridge. In other implementations, adding an identifying mark to the cartridge is performed before loading the patient sample into a sample port. In any case, patient and sample identification and marking occur prior to the inserting step.

### 1.4 Inserting Cartridge into Diagnostic Instrument

### 1.4.1 Overview of Insertion Step

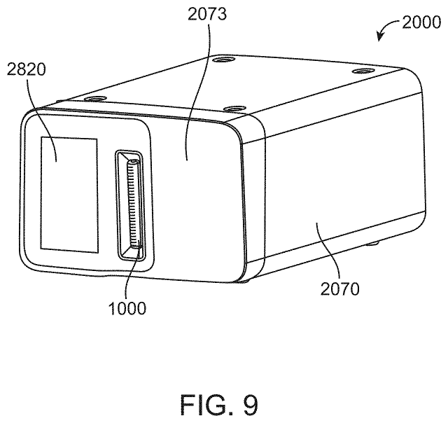

Another aspect of the invention provides inserting the cartridge into an opening of the diagnostic instrument. In some implementations, prior to inserting the cartridge into the diagnostic instrument, if required, the operator enters an identifying security code or interacts with an instrument graphical user interface to comply with security and operator identification procedures. According to other subject embodiments, the operator performs a cartridge handling movement to place the cartridge into an appropriate orientation to be accepted by the instrument. In such embodiments, the instrument and/or cartridge can include various features, e.g. a rail, protrusion, indent, or key, for ensuring proper cartridge orientation. Upon orientation, in some embodiments the cartridge may be inserted into the instrument such that the cartridge is substantially within the interior of the instrument. As used herein, the phrase “substantially within the instrument” describes embodiments where the cartridge is completely contained within the interior of the instrument, such that the cartridge is no longer visible to the user. Furthermore, “substantially within the instrument” additionally describes embodiments where the majority of the cartridge is contained within the interior of the instrument but remains visible to the user. In such embodiment, the instrument may indicate that the cartridge is in use, e.g. during a testing protocol, using a light or any other equivalent signal. In alternative embodiments where the cartridge is substantially within the instrument but remains visible to the user, the instrument may indicate the cartridge is in use by maintaining the cartridge in a position such that the user is prevented from grasping the cartridge for removal from the instrument. An example of such embodiment is shown in FIG. 9. In the illustrated example, a proximal end of the cartridge along a cartridge width axis is visible to the user from the outside enclosure 2070 of the instrument. However, the exposed edge of the cartridge is not sufficient to grip the cartridge for removal. In addition, the cartridge may be positioned within the instrument such that a portion of the cartridge is within a field of view of an instrument optical subsystem.

### 1.4.2 Security Features

In some embodiments, the instrument is configured with certain security features for limiting access to the instrument prior to inserting the cartridge. Specifically, in one embodiment a user may be required to authenticate the identity of said user prior to conducting a test or accessing information stored on the instrument. Authenticating procedures can verify the identity of authorized medical personnel and grant access to the instrument computer system for the running diagnostic tests and accessing patient testing information. The exemplary security features described herein may be used to ensure compliance with regulations for the protection of certain health information, e.g. compliance with the Health Insurance Portability and Accountability Act (HIPPA).

In various implementations, the user is required to interact with the instrument to provide information for granting access to the instrument computer system based on their identity. In some implementations, interaction with the instrument involves touching the instrument GUI for entering a security code to grant or deny access. In alternative implementations, interaction with the instrument involves scanning a badge or ID card with a barcode embedded with operator identity information to grant or deny access. For example, the user may insert said badge or ID card into an opening of the instrument to permit an optical subsystem to scan and read the embedded identify information. In another implementation, interaction with the instrument involves using near field communication systems, e.g. those used in access badges and security cards. Such near field communication systems include, but are not limited to, swipe, dip or contactless proximity. For example, a user may present within range of an instrument Radio Frequency Identification (RFID) reader a tag embedded in a RFID card to grant or deny access. Various interactions between a user and instrument may be implemented to provide other operator identification and security protocols.

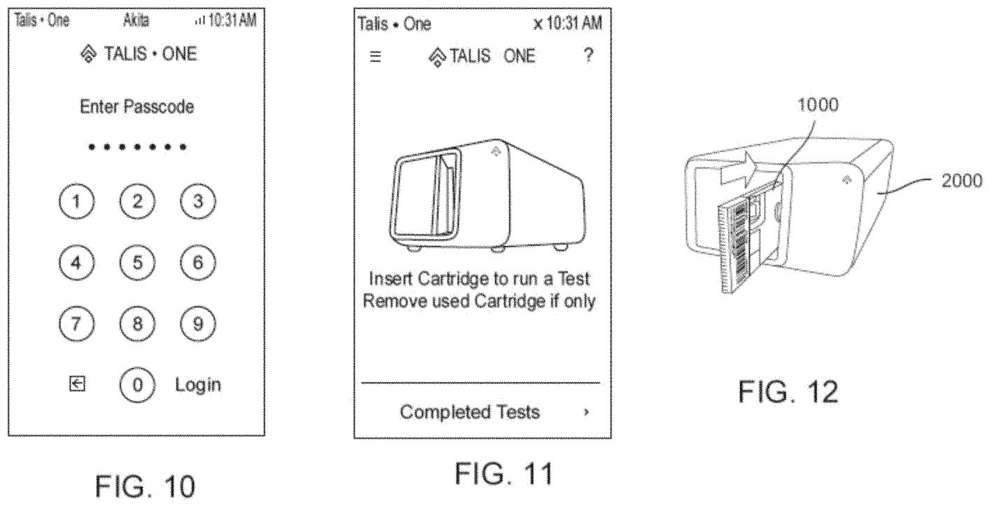

FIG. 10 is a view of an exemplary security screen on an instrument GUI. The user is required interact with, i.e. touch, the screen to enter a security code to unlock the instrument. The term “unlock” refers to the successful entering of a correct password to gain access to the instrument computer system via the GUI. Upon successful unlocking, the instrument GUI may be configured to display a variety of screens. For example, the GUI may display a start screen, as shown in FIG. 11, or other screens as described herein to the operator. In one implementation, to provide enhanced instrument security, the ‘Security’ screen (FIG. 10) or ‘Start Test’ screen (FIG. 11) is displayed for a predetermined time period of 5 minutes or less, 2 minutes or less, 1 minute or less, or 30 seconds or less. After expiration of the predetermined time period, the GUI can be configured to lock or “time out” by displaying an ‘Idle’ screen until further user interaction with the instrument and successful authentication is executed. In one implementation, the idle screen is a blank screen.

### 1.4.3 Orientation

During the inserting step, the operator performs a cartridge handling movement to position the cartridge into a preferred orientation to be accepted by the instrument. The single use integrated diagnostic cartridge is received and maintained within the instrument enclosure in the preferred orientation for the duration of the testing protocol until ejecting from the instrument. In some embodiments, the diagnostic cartridge and/or instrument may be configured with various guide features to prevent a user from inserting the cartridge in an incorrect orientation during insertion. Various exemplary features for facilitating insertion of the cartridge in the preferred orientation are described below.

Throughout the disclosure that follows, the term “vertical” position refers to the relationship of a testing cartridge to a vertical plane and a horizontal plane orientation provided by the design characteristics of a specific instrument embodiment. The vertical plane orientation is one allowing for the use of gravity for fluid movement for processing and handling steps performed during system operations. As such, terms of orientation such as higher and lower, upper and lower are understood in the context of gravitational flows of a generally vertical system orientation. In use, an instrument may be placed on a table or shelf that induces a tilt or incline to the instrument during use. Even though the instrument and cartridge are tilted during use this tilting up to and including +/−30 degrees is considered vertical as used herein. Moreover, tilting may be within the range of +/−15 degrees and also be considered vertical as used herein. Tilting within the above mentioned ranges would retain sufficient desired vertical orientation so as to maintain desired and expected gravity flow and characteristics.

In some embodiments, the instrument is adapted and configured to operate with cartridges configured to operate in such a vertical orientation. As such, the meaning of upright is that positioning of the cartridge relative to the components of the instrument while maintaining an orientation of the cartridge so as to operate the cartridge within the designed cartridge orientation principals. In one embodiment, upright refers to an orientation of the cartridge within the instrument to being vertical within the instrument. This is the orientation that is illustrated in the several views of the instrument in FIGS. 1, 8, 9, and 12. In the view of FIGS. 2 and 3, an arrow indicates the vertical orientation and points towards UP 1005. However, the operation and configurations of the instrument is not so limited. Based on variations in fluid flow characterizations of a specific single use cartridge, the orientation of the cartridge to the components of the instrument may be modified while still enabling the upright fluid flow principals implemented in a specific cartridge design. As a result, in other configurations, upright may include a slightly inclined orientation where the cartridge may be inclined relative to a vertical plane of the instrument while still providing the needed discrete actions of having an up and a down within the cartridge fluid schemes.

### 1.4.3.1 First Orientation

In many implementations, the cartridge is positioned in a first orientation while loading the sample and adding the identifying mark. As described herein, the cartridge dimensions are defined by its length, width, and height. Accordingly, each dimension has a respective associated axis, e.g. a cartridge length axis, a cartridge width axis, and a cartridge height axis. In one embodiment shown in FIGS. 2 and 3, the cartridge length axis 1020 and cartridge width axis 1025 lie within the plane of the page. Additionally, cartridge height axis is represented by a circle 1030 which is normal to the plane of the page. In one implementation, a first orientation for loading and adding the identifying mark is characterized by an orientation such that the cartridge height axis is normal to a work surface supporting the cartridge during the loading step or adding step, or is normal to a work surface supporting the instrument. In other words, the shortest dimension of the cartridge is normal to the work surface. In a further implementation, the first orientation is “horizontal” such that the cartridge length axis, i.e. the longest dimension, and cartridge width axis are within a plane parallel to the plane of the work surface, e.g. tabletop or countertop. Additionally, the cartridge length and width axes are in a plane parallel to a horizontal plane of the instrument. In some implementations, loading the sample into the cartridge comprises flowing a liquid sample into the sample port, wherein the cartridge is horizontally oriented. FIGS. 5-7 demonstrate the cartridge in the horizontal orientation prior to the user performing a cartridge handling movement and inserting into the instrument. As shown in FIGS. 5-7, the cartridge height axis, i.e. axis defining the thickness of the cartridge, is normal to the work surface, here a tabletop, on which the cartridge is positioned on. Additionally, in this orientation the cartridge length axis and width axis are parallel to the horizontal plane of the work surface and instrument. In the instant case, upon completion of adding the identifying mark (FIG. 4), the loading the sample (FIG. 5) and securing the cap 1181 (FIG. 6), the user is ready to perform a cartridge handling movement for inserting the cartridge into the instrument. The cartridge handling movement can be any movement performed by the user for positioning the cartridge into an appropriate orientation accepted by the instrument.

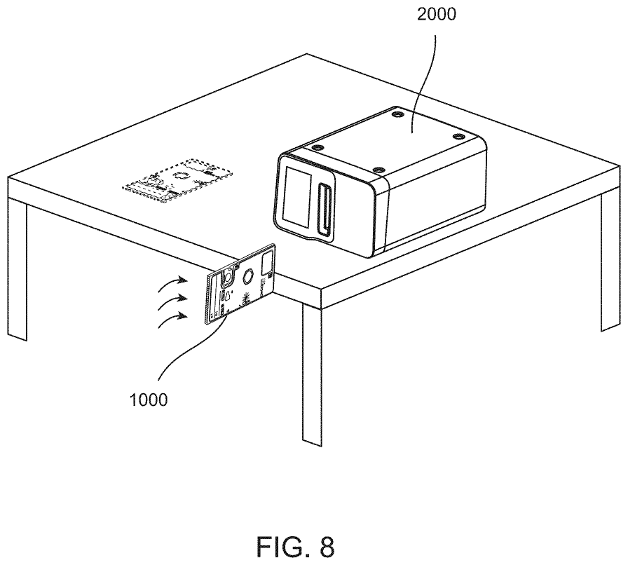

In some embodiments, the first orientation for loading and adding the identifying mark is different from a second orientation for inserting the cartridge into the instrument. Accordingly, said cartridge handling movement is any movement that transitions the cartridge from the loading and adding identifying mark orientation, i.e. the first orientation, to an inserting orientation, i.e. a second orientation. FIG. 8 illustrates an exemplary cartridge handling movement when the first orientation and second orientation are different. The user rotates the cartridge 90° along the cartridge length axis 1020 until the width of the cartridge is substantially normal to a horizontal plane of the tabletop. Additionally, the cartridge handling movement is performed until the cartridge is placed into an orientation for introduction into the instrument. In the illustrative embodiment of FIG. 8, the first and second orientations are generally orthogonal so the rotation is 90 degrees. Other cartridge handling movements are possible based on the first and the second orientations of a particular cartridge and instrument implementation. As further described herein, the cartridge and/or instrument can include one or more features, e.g. rails, protrusions, indents, or keys, for informing the user that the cartridge was rotated into a correct second orientation for insertion. A discussion regarding different orientations for inserting the cartridge into the instrument is described below.

In alternative embodiments, the first orientation for loading and adding an identifying mark is identical to the second orientation for inserting the cartridge. Such cartridge handling movement may simply be any movement that places the cartridge into the instrument without changing orientation.

### 1.4.3.2 Second Orientation

As described herein, the loading and adding an identifying mark orientation may differ from the inserting orientation. In various implementations, a second orientation is determined by an opening within the instrument enclosure for inserting the cartridge containing a patient sample. In various embodiments, the opening within the instrument enclosure is a hole, gap, space, slot, window, drawer, cabinet or any other aperture for permitting limited access to the interior of the instrument. The opening allows a user to insert the cartridge into the instrument to begin a testing sequence or testing protocol.

In one implementation, the instrument opening is a loading slot. In a preferred embodiment, the instrument opening is a loading slot wherein the loading slot is vertically oriented. The vertically oriented loading slot 2072 is shown in FIGS. 1, 7, 8, and 9. In the instant case, the vertically oriented loading slot is collinear with the cartridge width axis 1025. Accordingly, in this preferred embodiment, the instrument loading slot determines the second orientation of the cartridge. In one embodiment, moving the cartridge into the second orientation comprises rotating the cartridge about the cartridge length axis 1020. In a further embodiment, the second orientation is orthogonal to the first orientation, such that inserting the cartridge into an instrument occurs using a loading slot in the second orientation, i.e. vertical orientation. Based on specific cartridge geometry and cartridge-instrument interfaces, a wide range of cartridge handling movements are envisioned. Cartridge handling movement embodiments for translating a cartridge from a sample loading and identification marking orientation to an instrument loading or insertion orientation may include revolving a cartridge about one or more or a combination of rotations about or movements along a cartridge width axis, a cartridge length axis or a cartridge height axis.

In one implementation, a second orientation for inserting a cartridge into the instrument is characterized by an orientation such that the cartridge height axis 1030 is parallel to a work surface supporting the cartridge during the loading step or adding step, or supporting the instrument. In other words, the shortest dimension of the cartridge is parallel to the work surface. In such implementations, the cartridge length axis 1020 is normal to a rear wall of the instrument in the second orientation. Additionally, the cartridge width axis of the cartridge is normal to a base of the instrument when in the second orientation. Furthermore, the cartridge length axis is also parallel to the work surface supporting the cartridge during the loading step and the adding step. In another implementation, the second orientation is “vertical” such that the cartridge length axis and cartridge width axis are within a plane substantially perpendicular to the plane of the work surface, e.g. tabletop or countertop. In other words, the cartridge length and width axes are in a plane parallel to a vertical plane of the instrument.

### 1.4.4 Positioning

In some embodiments, the user inserts the cartridge into the instrument such that the cartridge is substantially within the interior of the diagnostic instrument. The positioning of the cartridge may coincide with an instrument optical subsystem. In one embodiment, the patient label area of the cartridge 1040 is within a field of view of an optical subsystem, enabling the capturing of an image of the patient label area. As described herein, the cartridge may be inserted in a position such that the field of view of the optical subsystem captures images of one or more machine readable codes. Additional description around the instrument optical system is described further herein.

### 1.5 Initiating Testing Protocol

### 1.5.1 Overview of Observing Step

According to another aspect of the invention, the method provides observing on the graphical user interface an indication of the type of test to be performed on the cartridge and an image of the identifying mark in the patient label area after inserting the cartridge into the instrument. In a further embodiment, the method further comprises observing the identifying mark on a graphical user interface of the instrument without touching the graphical user interface of the instrument or performing any other user interaction to contact the instrument after performing the inserting the cartridge step. In some implementations, while displaying the identifying mark for a predetermined time period, the user is given the opportunity to review patient and testing information. In some embodiments, the instrument testing sequence for conducting a diagnostic test is initiated after the predetermined time period without user interaction with the instrument. In an alternative embodiment, the instrument testing sequence for conducting a diagnostic test is imitated after the user interacts with the instrument. In another alternative embodiment, the user can abort the current test based on the information observed.

### 1.5.1.1 Information Confirmation

In various subject embodiments, the user observes an image captured of the patient label area presented on the instrument graphical user interface. Displaying the patient information on the GUI enables the user to review the patient information within the patient label area to confirm the information is correct. In further implementations, displaying the identifying mark further provides the user with information regarding the test type to be run on the cartridge. Accordingly, the user can observe and review the displayed test type and confirm the correct testing protocol or sequence will be run on the patient sample.

In some embodiments, after the inserting step, the identifying mark in the patient label area is displayed for a predetermined time, e.g. less than 2 minutes, less than one minute, less than 30 seconds, or less than 10 seconds. Alternatively, the predetermined time may be a time period inherently determined by the instrument default settings or may be a time selected by a system administrator. In one implementation, a testing protocol is automatically initiated when the predetermined time period has elapsed after completing the inserting the cartridge step. In an alternative implementation, the user interacts with the graphical user interface to initiate a testing protocol after the observing the image of the identifying mark and test type to be performed. In either case, a testing protocol initiates in the absence of a termination command executed by the user. In instances where the user observes an error in the displayed information, the user may cancel a testing protocol based on the image of the identifying mark on the instrument graphical user interface. The user may cancel the testing protocol by interacting with the GUI to execute a termination command. In implementations where the instrument GUI is configured as a touchscreen, the user cancels the testing protocol by interacting with a portion of the touchscreen. Accordingly, such termination command causes the cartridge to be ejected.

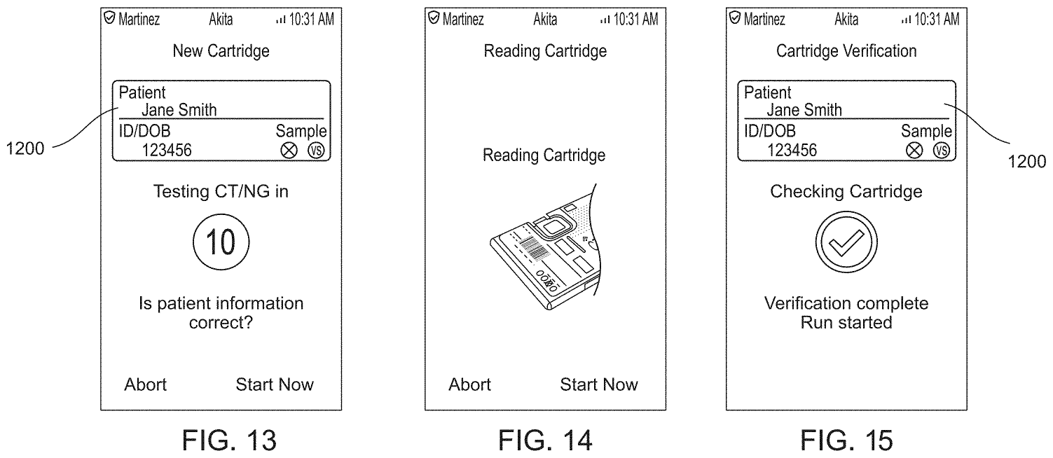

The ‘New Cartridge’ screen in FIG. 13 is an exemplary GUI displayed to the user for reviewing the patient identifying mark and test type. As shown, an image of the patient identifying mark 1200 is displayed together with a question prompting the user to confirm the displayed information. The exemplary GUI in FIG. 13 provides an opportunity for the operator to (a) confirm that the proper patient sample was inserted and (b) abort the run before the testing protocol begins. In the exemplary screen, the user is given 10 seconds to review the information. If any of the information presented in the GUI is incorrect, the operator may touch the ‘Abort’ portion of the GUI to cancel the test. Alternatively, the user may select the ‘Start Now’ portion, or equivalent, of the GUI to initiate the test and override the countdown timer of the predetermined period. In the absence of a user interaction with the GUI, the instrument will automatically begin upon expiration of the predetermined period.

### 1.5.2 Testing Sequence/Protocol

### 1.5.2.1 Verification Tests

In embodiments discussed further herein, the diagnostic instrument may be configured to perform a plurality of verification tests. Various cartridge, instrument, and sample verification tests can be implemented during the beginning portion of the testing protocol for confirming cartridge/instrument and sample integrity. As described further below, the verification tests performed during this step will vary depending on the specific instrument and cartridge designs implemented, as well as the sample type and amount needed for a proper testing sequence. In some embodiments, the user observes information related to verification testing displayed on the GUI while the instrument conducts the plurality of verification tests. Furthermore, in some embodiments, the user is provided an opportunity to cancel the test or initiate the test while observing such verification testing screen. FIG. 14 illustrates a ‘Reading Cartridge’ screen shown while the instrument conducts verification testing. Upon the successful completion, the user views updated information confirming successful verification testing and indicates that the testing protocol has begun. FIG. 15 illustrates a ‘Cartridge Verification’ screen displayed to the user after indicating the test run is initiated. Subsequently, the instrument executes a plurality of sample processing steps for performing a nucleic acid amplification for determining the presence or absence or quantity of a target pathogen.

### 1.5.2.2 Progress Timer

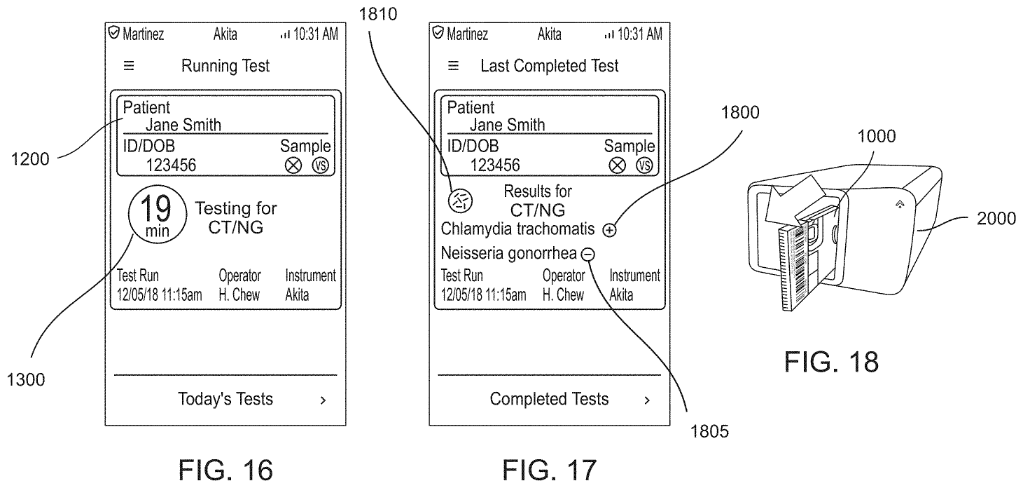

In some embodiments, the user observes a progress timer of the testing protocol with the time remaining displayed on the graphical user interface. In another embodiment, observing the progress timer may further include additional information associated with the currently conducted test. The additional information preferably includes at least the test type being conducted and/or the image of the patient identifying mark. Such additional information can further include, but is not limited to, the test run start date and time, operator identification, and instrument name. FIG. 16 demonstrates an exemplary ‘Test in Progress’ or ‘Running Test’ screen observed by the user while the testing sequence for a nucleic acid amplification is performed. As described herein, an image of the identifying mark 1200 in the patient label area 1040 of the cartridge is displayed. Below the image, a countdown timer 1300 indicates the remaining time for this test, e.g., a Chlamydia trachomatis (CT) and Neisseria gonorrhoeae (NG) test, is 19 minutes. Accordingly, in some embodiments, observing the identifying mark comprises waiting for the completion of the testing protocol. Additional operator interaction is not required during the testing sequence until the test is completed.

In various embodiments, while the user waits for the completion of a testing protocol, the user can obverse and review past results from previously conducted testing protocols conducted on the instrument. Accessing previous patient and testing information can require interacting with a portion of the graphical user interface. In embodiments where the graphical user interface is a touchscreen, the user may touch an indicated portion of the GUI to communicate with the instrument for accessing past test results. In some embodiments, the GUI can include icons or a string of text characters for prompting the user to interact with the specific portion. For example, as shown in FIGS. 11 and 17, a user can touch the bottom portion of the screen indicated by ‘Completed Tests’ to view all past test results conducted on the instrument. Similarly, in FIG. 16 a user can interact with the bottom portion of the screen denoted by ‘Today's Test’ to view all past test results from the current day. Upon interacting with a portion of the GUI to review past test results, a user observes a test result summary screen with results from previously conducted testing protocols. A detailed description of reviewing previous test results is described in greater detail below.

### 1.5.2.3 Privacy

As described herein, an instrument GUI can be configured to display an idle screen after a predetermined time period without operator interaction. In some embodiments, while displaying the identifying mark and progress timer during the testing protocol, the user observes the idle screen after a predetermined period without user interaction with the instrument. A time based idle screen can be implemented to protect patient privacy, such that limited or no access to the instrument is permitted until successful authentication is executed by user interaction with the instrument. In such embodiments, the idle screen is observed during the testing protocol after a predetermined period of five minutes, four minutes, three minutes, two minutes, one minute, or anytime therein. The idle screen displayed to the graphical user interface as a screen saver may be a blank screen, a static image, or an animated image. In a preferred embodiment, interacting with the instrument further comprises touching the graphical user interface to enter a security code after observing the identifying mark. For example, after the predetermined period without user interaction, the user observes a blank screen displayed by the instrument or while conducting the testing protocol. Upon user interaction, the operator may observe a ‘Security’ screen, as shown in FIG. 10 prompting the user to enter a security code prior to gaining access to patient testing information.

In one embodiment where the user gains access upon successful authentication, e.g. after observing an idle screen during the testing protocol, the user observes on the graphical user interface a progress timer of the testing protocol. In an alternative embodiment where the user gains access upon successful authentication, the user observes on the graphical user interface a listing of one or more test results based on previously conducted testing protocol. In another implementation where the user gains access upon successful authentication, the user observes on the graphical user interface a listing of one or more test results based on the most recently conducted testing protocol. In yet another implementation where the user gains access upon successful authentication, the user observes on the graphical user interface information prompting the user to begin a new diagnostic test.

### 1.6 Observing Results

### 1.6.1.1 Overview Observing Results

Limited user interaction occurs between the user and instrument after the instrument initiates a testing protocol to determine the presence or absence of a target pathogen, such that in many cases user interaction with the diagnostic system occurs upon cartridge ejection. In some aspects of the invention, the user observes an image of the identifying mark and an indication of a presence, an absence, or a quantity of a target pathogen in the sample on the graphical user interface. In some embodiments, the indication of a presence, an absence, or quantity of a target pathogen is observed without interacting with the instrument. In other embodiments, the indication of a presence, an absence, or a quantity of a target pathogen is displayed after the user interacts with the instrument. In such an embodiment, the user interacts with the graphical user interface to enter a security code prior to observing the indication of a presence, an absence, or quantity of the target pathogen.

### 1.6.1.2 Single Indicator for a Test Result

In various implementations, the user observes a single indicator representing a test result indicating a presence of a target pathogen, an absence of a target pathogen, or a quantity of the target pathogen in the patient sample. Accordingly, the single indicator may represent a positive test result or a negative test result. As used herein, the term “positive” sufficiently corresponds with a result from a testing protocol detecting the presence of a target pathogen. The term “negative” sufficiently corresponds with a result from a testing protocol detecting the negative presence, i.e. absence, of a target pathogen. In one implementation, the single indicator is colored differently depending on whether it is reporting a positive or negative result. In a preferred embodiment, the single indicator for a positive test result is red. In another preferred embodiment, the single indicator for the negative test result is green. In an alternative implementation, the single indicator is a colorblind-adapted equivalent. In some implementations, the single indicator is an image, an icon, a glyph, or any other visual representation. In other implementations, the single indicator comprises a number of text characters.

FIG. 17 illustrates a ‘Last Completed Test’ result screen after the completion of an instrument testing sequence. The exemplary screen displays an image of the patient identifying mark 1200 and an assay summary containing test results, i.e. the indication of a presence, an absence, or a quantity of a target pathogen. As shown, a single indicator representing each one of the test results, e.g. a test result for Chlamydia trachomatis (CT) and a test result for Neisseria gonorrhoeae (NG), is an icon 1800 and 1805, respectively. Specifically, the icon representing a positive test result is denoted by a positive (+) sign 1800 and the icon representing a negative test result is denoted by a negative (−) sign 1805. In such implementation, the operator is quickly alerted to which one of the one or more test results conducted on a single cartridge is positive. The exemplary icons indicate the patient sample is positive for detecting Chlamydia trachomatis (CT) and negative for detecting Neisseria gonorrhoeae (NG).

### 1.6.1.3 Single Indicator for Two or More Test Results (Results Summary Icon)

In another embodiment, a single indicator represents a result for two or more tests performed on a single cartridge. In one embodiment, the single indicator represents a negative presence of all target pathogens from two or more tests. In a further embodiment, the single indicator represents a positive presence of at least one target pathogen from the two or more tests. In some implementations, the single indicator representing the plurality of test results is colored. In a preferred embodiment, the single indicator for positively detecting at least one target pathogen is red and the single indicator for negatively detecting the presence of all target pathogens is green. In an alternative embodiment, the single indicator for representing a plurality of test results is colorblind-adapted equivalent. In one implementation, the single indicator representing a totality of test results is an image, an icon, a glyph, or any other visual representation. In other implementations, the single indicator representing the plurality of test results comprises a number of text characters. Note, in such embodiments, the single indicator representing the totality of the two or more test results is an indicator different from those used to represent a singular test result described above. In other words, a single indicator can be used to represent a summary of all test results conducted on a single cartridge.

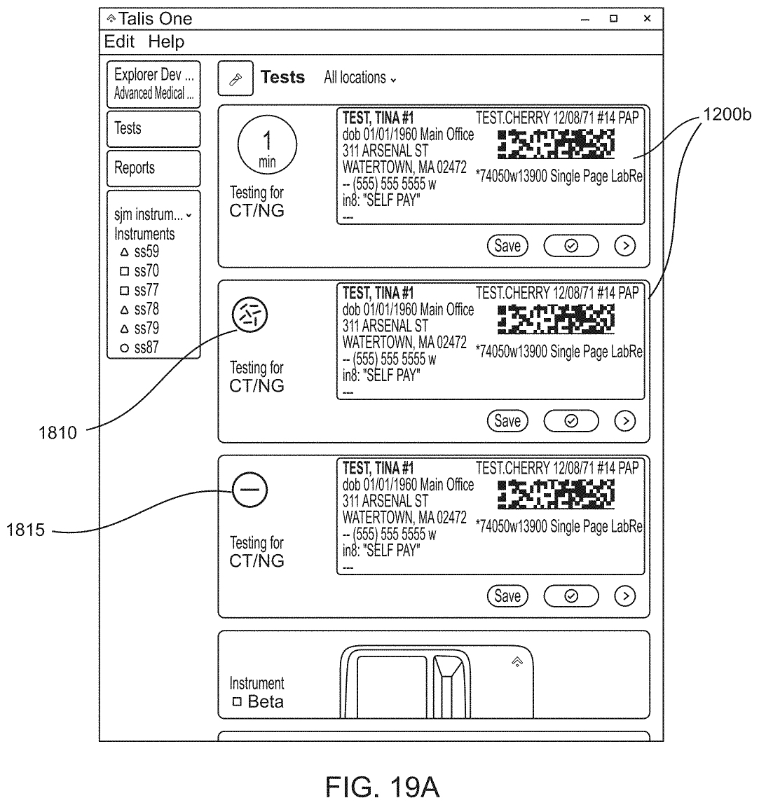

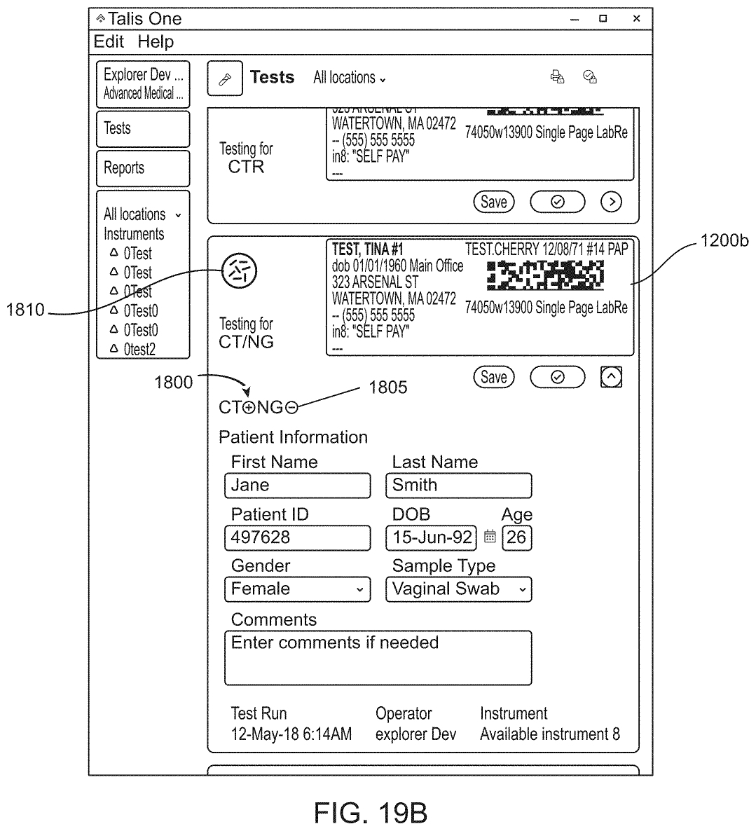

Returning to the ‘Last Completed Test’ result screen shown in FIG. 17, the single indicator representing the totality of the two tests, i.e. both CT and NG, is a ‘Results Summary’ icon. The ‘Results Summary’ icon 1810 displayed in FIG. 17 represents the positive presence of at least one target pathogen among the plurality tests conducted on the cartridge. By way of example, the ‘Summary Results’ icon 1810 for positively detecting at least one target pathogen, i.e. Chlamydia trachomatis, is characterized by a circle with line segments arranged randomly therein. In another example, the ‘Summary Results’ icon for negatively detecting the presence of all target pathogens is characterized by a circle with a negative (−) sign therein, as shown in FIG. 19A. The embodiments describing the single depiction of a plurality of results provides the advantage of a simplified representation in a clear and concise manner, thus allowing an operator to quickly identify whether a patient needs a course of treatment. A further discussion of the simplified and time saving benefits are described further herein.

### 1.6.1.4 Timing of Observing Result

In various embodiments, the user observes the image, the icon, or the glyph on the graphical user interface indicating presence, absence, or quantity of a target pathogen less than 60 minutes, less than 25 minutes, less than 20 minutes, less than 15 minutes, or less than 10 minutes after inserting a cartridge into the instrument. In an alternative embodiment, the user performs a single interaction with the instrument to view the test result after observing an image of the identifying mark on the graphical user interface after a time delay of at least 15 minutes or a time delay of between 10 minutes to 15 minutes.

In some embodiments, the user observes an image, an icon, or a glyph on the graphical user interface indicating a result of a testing protocol performed on the sample while removing the cartridge from the instrument. In another embodiment, the user observes an image, an icon, or a glyph on the graphical user interface indicating the result of a testing protocol performed on the sample after removing the cartridge from the instrument. In yet another embodiment, the user observes an image, an icon, or a glyph on the graphical user interface indicating a result of a testing protocol performed on the sample before removing the cartridge from the instrument. In another implementation, the user observes an image of the identifying mark and the indication of the test result while removing the cartridge from the instrument. Alternatively, the user observes an image of the identifying mark and the indication of the test result after removing the cartridge from the instrument. In yet another alternative implementation, the user observes an image of the identifying mark and the indication of the test result before removing the cartridge from the instrument. Such implementation may be applicable where an instrument testing protocol has been completed and the graphical user interface displays an idle screen after a predetermined time period without user interaction. Accordingly, as described further herein, the user must interact with the graphical user interface to enter a security code to view the image of the identifying mark and indication of the test. This action to observe the identifying mark and test result may be performed before removing the cartridge.

### 1.6.1.5 Privacy of Result

As further discussed herein, a diagnostic instrument may be configured with a time based idle screen after a predetermined time period without user interaction to protect patient information. For example, the instrument graphical user interface may display the idle screen during the testing protocol. In cases where the instrument completes the testing protocol while displaying the idle screen, the user may be required to interact with the instrument to access the results of the testing protocol. Specifically, the user observes an indication of a presence, an absence, or a quantity of the target pathogen after interacting with a graphical user interface to display the test result. In a preferred embodiment, the user enters a security code on the graphical user interface to observe the indication of a presence, an absence, or a quantity of the target pathogen. Alternatively, the user observes the indication of the presence, absence, or quantity of the target pathogen without touching the graphical user interface or otherwise interacting with the instrument after inserting the cartridge into the instrument.

### 1.7 Removing Cartridge from Instrument

### 1.7.1.1 Overview of Removing Step

A further aspect of the invention provides removing the cartridge from the instrument after the cartridge is automatically ejected from the instrument. FIG. 18 illustrates a diagnostic instrument 2000 ejecting a cartridge 1000 from the instrument prior to the user removing the cartridge. Further description regarding instrument ejection mechanisms are further discussed below. As described herein, the user may observe a test result before, during, or after removing the cartridge from the instrument. Alternatively, the user may observe an error message displayed on the graphical user interface before removing the cartridge from the instrument. In another alternative embodiment, the user may observe an error message displayed on the graphical user interface while removing the cartridge from the instrument. In yet another alternative embodiment, the user observes an error message displayed on the graphical user interface after removing the cartridge from the instrument. After removing the single use cartridge, the user discards the cartridge and can either review the most recent test result, review past test results from previously conducted testing protocols, or begin a new test. It is to be appreciated that an observed error message may relate to one or more of the early, automatic fault detection or confirmation operations discussed herein related to a sample, an integrated cartridge, an interface between an integrated cartridge and a diagnostic instrument or a diagnostic instrument itself.

### 1.8 Reviewing Past Results

In various aspects of the invention, the user can observe a list of past results from previously conducted testing protocols conducted on the instrument. Accessing previous patient and testing information can require interacting with a portion of the graphical user interface. As described herein, in embodiments where the graphical user interface is a touchscreen, the user can touch an indicated portion of the GUI to communicate with the instrument for accessing the list past test results. In some embodiments, the GUI can include icons or a string of text characters for prompting the user to interact with the specific portion. For example, as shown in FIGS. 11 and 17, a user touches the bottom portion of the screen indicated by ‘Completed Tests’ to view all past test results conducted on the instrument. Likewise, in FIG. 16 a user interacts with the bottom portion of the screen denoted by ‘Today's Test’ to view all past test results from the current day.

Upon interacting with a portion of the GUI to review past test results, a user observes a test result summary screen with a list of results from previously conducted testing protocols. FIG. 19A is an example of a test result summary screen observed by the user. Accordingly, a plurality of patient identifying marks 1200 and associated test results are viewed. As shown in the exemplary test result summary screen, the images of the identifying marks within the patient label area are printed labels 1200b containing both human readable information and information embedded in a machine readable barcode. In some embodiments, the interaction with the GUI can further comprise interacting with the graphical user interface to scroll through a list of past test results, thus enabling a user to find desired results for a specific patient. In another embodiment, the user may observe testing information relating to a currently conducted test in addition to previously conducted tests. As illustrated in FIG. 19A, the user observes an uppermost panel displaying information indicating that a currently conducted CT/NG test has 1 minute remaining in the testing protocol. Thereafter, a middle panel and bottom panel, with identifying marks and results for respective CT/NG tests, are further viewed below.

In some embodiments, the user may interact with the GUI to sort the list of past test results. For example, past test results can be sorted chronologically, by test type, user/operator name, positive results, negative results, or any other field associated with tests and/or results.

As described herein, test results can be represented by a single indicator corresponding to the total absence of all target pathogens or the presence of at least one target pathogen, e.g. a results summary icon. In FIG. 19A, the presence of at least one target pathogen is indicated with an icon 1810, e.g. a ‘Positive’ summary icon, characterized by a circle with line segments arranged randomly therein. The absence of all target pathogens is indicated with an icon 1815, e.g. a ‘Negative’ summary icon, characterized by a circle with a minus (−) sign therein. In another implementation, a user may further interact with the graphical user interface to display individual results of each of the two or more tests performed on the point of care cartridge. In such embodiment, the user may be motivated to further interact with the GUI to display individual results based on observing the single indicator, i.e. results summary icon. For example, the user may opt to interact with the GUI after viewing a positive summary icon 1810. Upon interaction, in some embodiments, the user observes detailed information characterizing individual test results of each target pathogen tested on the single cartridge.

Returning to the exemplary test result summary screen in FIG. 19A, upon viewing the positive summary icon 1810 in the middle panel, a user selects the middle panel by interacting, e.g. touching, the respective portion of the GUI. Such interaction prompts the display of additional testing information. An example of a screen displaying additional testing information is shown in FIG. 19B. Accordingly, the interaction with the GUI 2820 enables the user to observe the patient identifying mark 1200b and an indication of which of the two or more target pathogens tested resulted in the positive summary icon. As illustrated in FIG. 15B, the patient, Jane Smith, tested positive for Chlamydia trachomatis (CT) and negative for Neisseria gonorrhoeae (NG). The positive and negative test results are denoted by positive and negative single indicators 1800 and 1805 respectively, resulting in the display of positive summary icon 1810.

The single depiction of a plurality of test results provides the advantage of a simplified representation in a clear and concise manner. In this embodiment, the single indicator allows an operator to quickly differentiate patients who require a course of treatment from those who do not. Specifically, observing the negative summary icon, such as icon 1815 in FIG. 19A, notifies the user or operator, i.e. a healthcare worker, that no additional interaction with the instrument is necessary. Accordingly, the healthcare worker may inform the patient no treatment is required and dismiss the patient. Alternatively, viewing a positive summary icon, like icon 1810 shown in FIG. 19A-B, alerts the user that further interaction with the instrument is necessary to identify which target pathogen has been detected, such that an appropriate course of treatment can be administered.

### 1.8.1 Privacy of Past Results

As previously described, the instrument may be configured to display an idle screen after a predetermined period without user interaction. Accordingly, in some implementations, the user performs a single interaction to observe the indication of a presence or absence based on the testing sequence. In such instances where a testing protocol sequence is completed while the instrument displays the idle screen, the user may be required to perform a single interaction further comprising entering a security code to enable use of the graphical user interface to access testing information and test results. The requirement of entering a security code prior to accessing testing information protects patient health information and ensures compliance with regulations for the protection of certain health information. In some embodiments, if the user observes an idle screen and enters a security code within a predetermined time period after the completion of the testing protocol, the user observes a ‘Last Completed Test’ screen, as shown in FIG. 17. Otherwise, in alternative embodiments, if the user observes the idle screen and enters a security code after a predetermined time period after the completion of the testing protocol, the user observes a start screen, as shown in FIG. 11. In many embodiments, the predetermined time period is 5 minutes or less, 2 minutes or less, 1 minute or less, or 30 seconds or less. In some embodiments, the single interaction performed by a user may be the use of a near field or non-contact security device assigned to that user for accessing an instrument or results stored on an instrument.

In some implementations, if the user enters a security code within a predetermined time period of viewing the idle screen, the user observes a test result screen such as the one illustrated in FIG. 17. Otherwise, in alternative embodiments where the user enters a security code after the predetermined time period, the user may observe a start screen, as shown in FIG. 11. In such embodiment, the user can interact with the GUI to access the previous test result by selecting ‘Completed Tests’ section of the screen.

### 1.9 Instrument Set Up

### 1.9.1 Running Positive/Negative External Controls

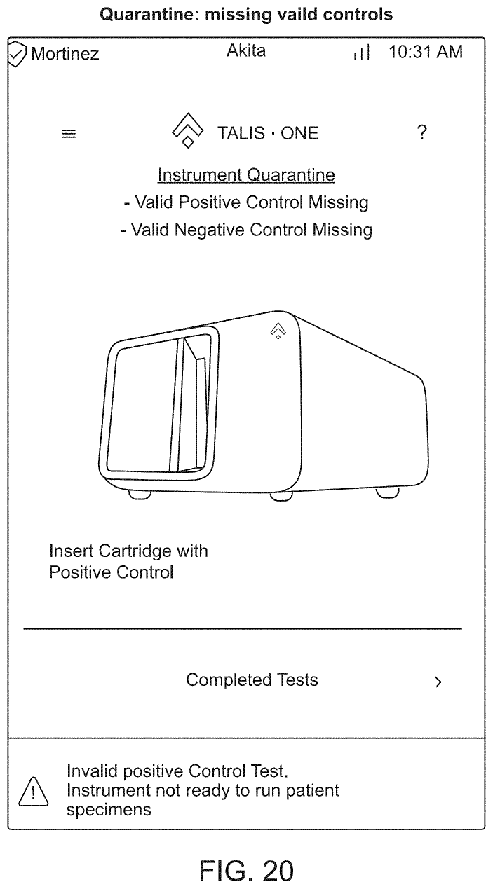

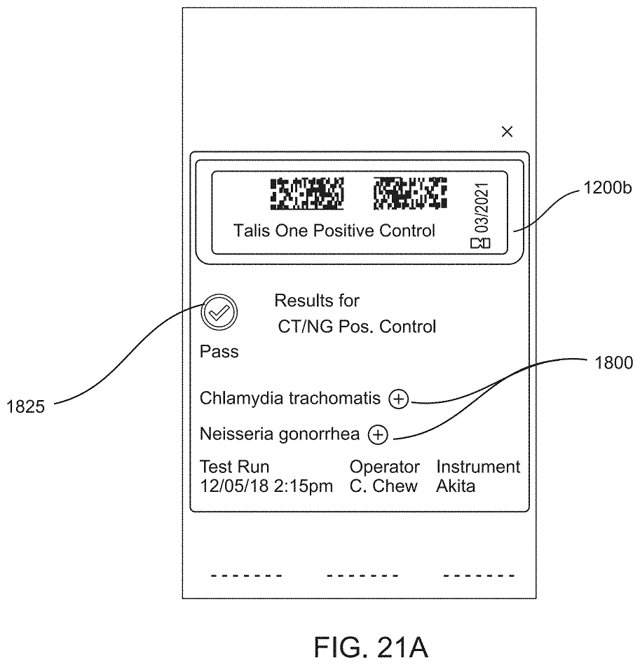

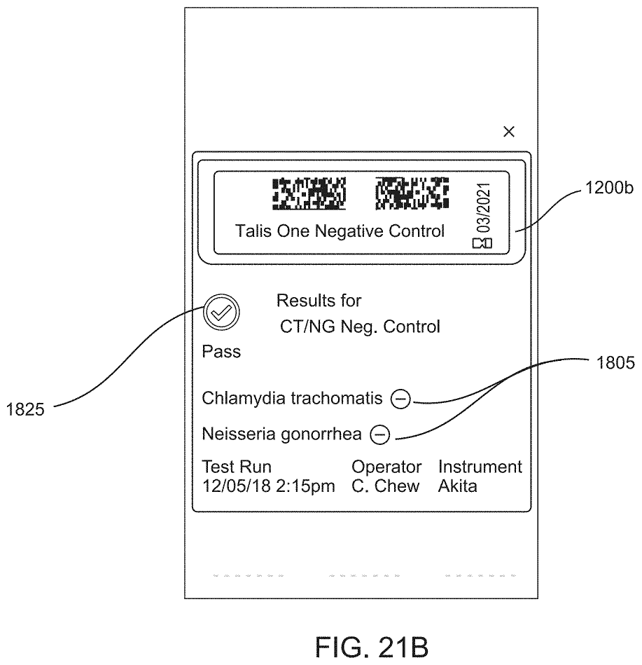

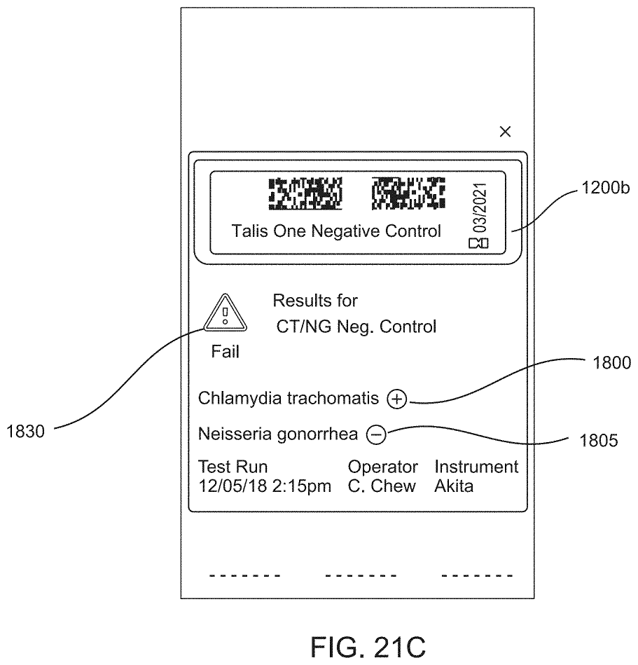

In some embodiments, the diagnostic instrument may be configured to require a user to run a positive and/or negative external control prior to running a patient sample suspected of containing a target pathogen, such that an instrument will refuse to run a patient sample until control testing is complete. In other embodiments, the instrument permits a user to run an external control at anytime that the instrument is unoccupied, e.g. to facilitate user training or as part of a standard lab facility qualification or maintaining certifications. External controls are useful in determining proper intended function of the instrument, cartridge, and assay by generating a predetermined result. When the expected result is reported, one or more aspects of the diagnostic assay, instrument, or cartridge are confirmed to be working as intended, thus enabling the user to assess a patient sample suspected of containing a target pathogen with confidence in the test result. When the expected result is not reported, the user is alerted that one or more aspects of the assay, instrument or cartridge is not working as intended. Therefore, if one or more aspects are unsatisfactory the user is prevented from running a patient sample. For example, the external controls may qualify and test an instrument or a new lot of assays against a known sample. Running positive and negative external controls is illustrated in FIGS. 20-21C.

In certain implementations, after successfully setting up the diagnostic instrument or adding a new user and/or a user group, a user may be prevented from testing a patient sample until successfully completing a plurality of external control tests. In some implementations, the user observes an instrument quarantine screen, like the one shown in FIG. 20, with information prompting the user to insert a cartridge containing a positive control or a negative control. The exemplary screen displays an icon indicative of an error or warning instructing the operator that the instrument is not ready to run a patient sample. As shown, this is attributed to the two missing controls tests, e.g. a positive and negative control. The user may proceed according to the methods described above. Specifically, the user must load the external control into a cartridge, close the sample port assembly, and insert the cartridge in the preferred orientation accepted by the instrument. Optionally, in some embodiments, the user can affix a pre-printed label or otherwise identify the test as an external control. Upon receiving the cartridge, the instrument executes an appropriate testing protocol, indicated by the one or more machine readable codes present on the cartridge, to determine the result of the positive and/or negative control. In some implementations, the user may further interact with a portion of the graphical user interface, while displaying the instrument quarantine screen, to access previous test results. As similarly described regarding FIGS. 19A and 19B, such interaction with the GUI will present the user with a test result summary screen displaying a list of past testing information.

Upon insertion and a successful testing sequence, in some embodiments, a user observes a positive control screen for a successful positive control run in detecting one or more target control pathogens. An example of a Positive Control screen is shown in FIG. 21A, which illustrates the positive detection of target control pathogens Chlamydia trachomatis (CT) and Neisseria gonorrhoeae (NG) denoted by a single indicator. As described herein, the single indicator can be an icon. In such case, a user observes the positive icon 1800 characterized by a circle containing a positive (+) sign therein located adjacent to each of CT and NG. In a further embodiment, the user observes a single indicator representing a passing result for detecting the presence of one or more target control pathogens. The single indicator 1825 shown in FIG. 21A is represented by an icon characterized by a circle containing a check mark therein.

Upon insertion and a successful testing sequence, in other embodiments, a user observes a negative control screen for a successful negative control run in detecting the total absence of all target control pathogens. An example of a Negative Control screen is shown in FIG. 21B, which illustrates the negative presence, i.e. absence, of target control pathogens CT and NG denoted by a single indicator. A user observes the negative icon 1805 characterized by a circle containing a minus (−) sign therein located adjacent to each of the CT and NG. In a further embodiment, the user observes a single indicator representing a passing result for detecting the absence of all target control pathogens. In some embodiments, the single indicator representing a passing result for detecting the absence of all target control pathogens is the same as the single indicator representing a passing result for detecting the presence of one or more target control pathogens. In other embodiments, the single indicator representing a passing result for detecting the absence of all target control pathogens is different than the indicator representing a passing result for detecting the presence of one or more target control pathogens. As shown in FIG. 21B, the icon 1825 representing a passing negative control run is the same as the icon representing a passing positive control run shown in FIG. 21A. Accordingly, after successfully completing all control tests, the user may observe a start screen as shown in FIG. 11.

In alternative embodiments where the instrument fails a positive or negative control test, the user observes a failed control screen. The failed control screen is observed by the user if the instrument fails to detect all target control pathogens during a positive control test, thus indicating one or more aspects relating to the assay, instrument, or cartridge are not functioning properly. Additionally, the failed control screen is observed by the user if the instrument detects at least one target control pathogen during a negative control test, thus indicating potential amplicon contamination. In either case, the user is prevented from running a patient sample until the instrument has satisfied all control tests. The user is required to insert additional control cartridges into the instrument to qualify the assay, instrument, or cartridge against the control tests.

In some embodiments, the user observes a single indicator representing a failed result for detecting the absence of all target control pathogens. In other embodiments, the user observes a single indicator representing a failed result for detecting the presence of one or more target control pathogens. In further embodiments, the single indicator representing the two failed results for a positive and negative control run are the same. In alternative embodiments, the single indicator representing the two failed results for a positive and negative control run are different. FIG. 21C is an example of a failed control screen during a negative control run observed by the user. As shown, the user observes an error icon 1830, characterized by a triangle with an exclamation mark contained therein, to alert the user of a failed control run. Specifically, the failed control icon was caused by the positive detection of CT. Note, in each of the exemplary control screens shown in FIGS. 21A-C, the identifying marks are printed labels featuring a machine readable barcode identifying the test runs as external controls.

### 2 Point of Care Instrument Operations

### 2.1 Overview of Instrument Configured to Run Molecular Diagnostic Test and Provide Easy User Workflow

The embodiments below relate to a diagnostic instrument used for performing methods of operating an instrument for testing a sample suspected of containing a target pathogen, as described herein. The various embodiments of a diagnostic instrument presented are adapted and configured to accept and process samples using any of a wide array of different testing methodologies and sample types. It is to be appreciated that an ordinary person skilled in the art may design and configure a diagnostic instrument for performing a molecular diagnostic test according to a variety of methods. Accordingly, the embodiments and configurations described below are meant solely for the purposes of clarity and understanding. Therefore, a person of ordinary skill may achieve the same desired objective of detecting nucleic acids indicative of a target pathogen using alternative methods, mechanisms, instrumentations, apparatuses, and systems other than those described herein. The advantageous methods of using a diagnostic system described and in the appended claims remain applicable to a variety of alternative diagnostic instruments, cartridges, and configurations.

In various implementations, the diagnostic instrument may be configured with a variety of assemblies and subsystems for operating a diagnostic instrument with minimal user interaction while generating reliable diagnostic results. One or more sample processing, amplification, and/or detection steps can be automated using a combination of assemblies, subsystems, and an appropriate computer control system to determine a diagnostic result. However, it is most preferable and advantageous to automate all sample processing, amplification, and detection steps to facilitate the ease of use for the user/operator by minimizing the number of steps performed by the user. For example, leveraging a diagnostic instrument capable of automating a molecular testing protocol may simply require a user to load a patient sample into a cartridge and insert said cartridge into the instrument. Upon insertion, the diagnostic instrument may execute a plurality of sample processing, amplification, and detection steps to arrive at a diagnostic result without any and/or minimal user interaction. Accordingly, the automation of such molecular testing protocol allows a lay or untrained user to perform a diagnostic test while simultaneously minimizing the risk of an erroneous result due to human error. As a result, embodiments of such automated and minimal and/or no user interaction aid in accomplishing one or more of the recommendations, guidelines and requirements above.

By way of introduction, FIGS. 1 and 7-9 illustrate an exemplary diagnostic instrument 2000 configured to be used with the various testing methods described herein. The exemplary instrument 2000 is shown in an exploded view in FIG. 24. The various embodiments of the instrument 2000 described herein are adapted and configured to accept and process samples using any of a wide array of different testing methodologies and sample types. Accordingly, instrument 2000 is configured with a mechanical subsystem, a pneumatic subsystem, a thermal subsystem, and an optical subsystem to execute sample processing and amplification steps for the detection of nucleic acids from one or more target pathogens.

Assemblies may vary based on the specific configuration of an instrument configuration and cartridge design. As such, an instrument 2000 may be configured to accept an integrated diagnostic cartridge of different configurations. The large number of different cartridge configurations lead to a similar number of complementary instrument designs with assemblies and subsystems adapted and configured for use with those particular cartridge designs. It is to be appreciated that despite the variety of instrument and cartridge configurations, the methods described herein of using a diagnostic system for performing rapid molecular diagnostic testing provides advantages, e.g. time saving benefits and user workflow simplification, may be implemented by medical or lay personnel operating in a point of care environment using alternative diagnostic systems.

### 2.2 Receive Cartridge

The diagnostic instrument typically comprises an outer enclosure, shell, cover, or any other housing for enclosing the instrument internal hardware from the user or operator. For example, enclosure 2070, shown in FIGS. 1, 9, and 24, encompasses all of the various subsystems and assemblies used to perform the various operations of a particular sample-cartridge-instrument combination when performing a molecular diagnostic test. In some embodiments, the enclosure of the diagnostic instrument may include an opening for receiving the cartridge and providing a user with limited access to the interior of the instrument. The opening can be any of a hole, gap, space, slot, window, drawer, cabinet or any other aperture for receiving the cartridge. As shown in FIGS. 1, 7-9, 12, and 18, the opening of the exemplary instrument is a loading slot 2072. As described herein, the instrument opening can define a unique orientation for receiving a cartridge inserted by the user. The loading slot 2072 is configured in a vertical orientation, thus requiring the user the orient the cartridge into a respective vertical orientation, as previously described herein.

In some embodiments, the enclosure of the instrument may be configured to support a display or graphical user interface (GUI) 2820 for providing a user with information and other interaction abilities for communicating with the instrument. In one embodiment, the graphical user interface is a touch screen such that the user may interact with the instrument by touching the screen or icons displayed thereon to access information and communicate with the instrument computer system further described herein.

### 2.2.1 Mechanical Subsystem

In some embodiments, a diagnostic instrument contains a mechanical subsystem for performing a variety of functions including, but not limited to, receiving the cartridge, clamping the cartridge, performing verification tests and/or establishing cartridge to instrument interfaces. The mechanical subsystem orchestrates the various processes conducted on the sample to arrive at the indication of a target pathogen. As further described herein, the mechanical subsystem can comprise a loading assembly for receiving and ejecting an inserted cartridge via the instrument opening, e.g. the loading slot. Furthermore, the mechanical subsystem can comprise a clamping subsystem used to initiate a plurality of instrument-to-cartridge interfaces for interacting with the cartridge. In some embodiments, the clamping subsystem supports various subsystems and assemblies for executing verification checks to determine instrument, cartridge and sample integrity prior to executing a testing protocol. Additionally, the mechanical subsystem may be configured to support additional assemblies and subsystems, such as a thermal subsystem and optical subsystem, for facilitating other sample processing and amplification steps on the cartridge.

### 2.2.1.1 Loading Assembly

In one embodiment, an instrument mechanical subsystem provides a loading assembly 2230 configured to receive a cartridge inserted into instrument 2000 by the user. Additionally, the loading assembly is configured to eject the cartridge upon completion of a diagnostic testing protocol without interaction by the user. FIG. 26A-B illustrate views of an exemplary loading assembly 2230 within instrument 2000. In one implementation, the loading assembly comprises rails 2231, rack 2232, pinion 2233, pusher carriage 2234, spring 2235 and a load position sensor 2236.

One technique to enable ease of use and reduction of error involves simplification of the cartridge-instrument interface. In one aspect, the cartridge and instrument may include one or more features used to ensure proper cartridge orientation for insertion with the instrument. Interference features and guides may be used for this purpose to ensure that only a properly oriented cartridge will be accepted into the instrument. One exemplary implementation of such features are shown in the FIGS. 25, 26A and 26B. The loading assembly 2230 allows a cartridge to ride along two rails 2231 until a distal end of the cartridge contacts pusher carriage 2234 after being inserted into the instrument via the loading slot. The cartridge is permitted to move along rails 2231 until pinion 2233 reaches the end of rack 2232. As the cartridge contacts the pusher carriage 2234 and travels along two rails 2231, spring 2235 is stretched out of an equilibrium position until the cartridge is no longer permitted to travel along the rails and flag 2237 triggers load position sensor 2236. The triggering of the load position censor confirms the cartridge is substantially inserted into the instrument. The cartridge remains in this position, with the load position sensor 2236 triggered, until the completion of the testing protocol during normal instrument operation, until the cartridge fails one or more verification tests, or until a user performs a termination command. In any case, the cartridge will remain in this position until ejected by the loading assembly.

Upon completing a testing protocol, failing a verification test, or receiving a termination command by the user the loading assembly ejects the cartridge. As further described below, an instrument clamping subsystem may unclamp the cartridge enabling spring 2235, along the bottom rail, to relax towards the equilibrium position, thus ejecting the cartridge. An external view of the instrument ejecting a cartridge is illustrated in FIG. 18.

### 2.2.1.1.1 Guide Feature (Orientation)

To ensure that a lay or untrained user can properly orient the cartridge when inserting it into the instrument, the cartridge and instrument preferably comprise complementary loading and orientation guide features. One implementation of loading assembly guide features is illustrated in FIGS. 27A-28B. Accordingly, in one embodiment, an upper rail 2231a and lower rail 2231b comprise a guide feature 2240 for properly aligning and maintaining a cartridge in a preferred orientation. As described herein, the preferred orientation is a vertical orientation dictated by the vertically oriented instrument loading slot. In another embodiment, the width of a rail gap corresponds to a thickness of either a portion of or the entire cartridge, e.g. along the cartridge height axis 1030, such that a cartridge is permitted to be inserted when at least a portion of the cartridge is within the width of the rail gap. Additionally, it is to be appreciated that features on the cartridge can used to ensure proper cartridge orientation by interfering with at least one part of the instrument if inserted in the incorrect orientation. Accordingly, if positioned in the correct orientation, at least one part of any designed gap or spacing formed or partially formed within the cartridge will align with corresponding instrument features. Such implementation is further described below.

A cartridge inserted with proper alignment is shown in a top down view in FIGS. 27A-B and shown in a bottom up view in FIGS. 28A-B. FIG. 27A illustrates the distal end of the cartridge prior to being inserted into the loading assembly 2230 and prior to interacting with upper guide feature 2240. FIG. 27B shows a cartridge during loading with upper guide feature 2240 in alignment with the cartridge gap or spacing formed or partially formed therein. The gap or spacing formed in the cartridge is configured to interface with the upper guide feature 2240 to direct the cartridge along the upper rail 2231a. In one implementation, an interference feature 1022 is formed within the cartridge, as shown in FIGS. 28A and 28B. FIG. 28A illustrates the distal end of the cartridge, in a bottom up view, prior to being inserted into the loading assembly 2230 and prior to lower guide feature 2240 interacting with interference feature 1022. FIG. 28B shows a cartridge during loading with a lower guide feature 2240 in alignment with interference feature 1022. Such an interference feature in combination with loading assembly guide features ensures proper alignment and prevents a user from inserting the cartridge in an incorrect orientation, e.g. 180° rotated along the cartridge length axis 1020. Similarly, the grip on the proximal end of the cartridge (See FIGS. 2, 4-6, and 27A-28B) discourages insertion 180° rotated about the shorter axis, i.e. the cartridge width axis 1025, and the lack of a rail at the grip end disallows insertion in such orientation.

### 2.2.1.2 Clamping Subsystem

As previously described herein, a combination of assemblies, subsystems, and an appropriate computer control system can be used to automate a plurality of steps in a testing protocol to minimize the user interaction with the instrument. Upon cartridge insertion, an instrument computer control system may cause the instrument to automatically engage a clamping subsystem to immobilize the cartridge in a position for conducting the testing sequence. FIG. 30 illustrates an exemplary computer control system and is further described in greater detail below. In one embodiment, the computer control system initiates a clamping sequence after triggering a load position sensor contained within the loading assembly. The clamping sequence initiates critical interfaces between the instrument and corresponding cartridge components, such that the plurality of instrument assemblies and subsystems may interact with the cartridge to perform a testing protocol. Accordingly, the computer control system autonomously operates each of the plurality of assemblies and subsystems thus simplifying the operation of the instrument for the user.

In one embodiment, the clamping subsystem comprises a fixed bracket assembly and a moving bracket assembly for maintaining the cartridge in a secure and preferred orientation. The fixed bracket assembly 2010 is the stationary component of the clamping subsystem located within the instrument. Complementary to the fixed bracket assembly is a moving bracket assembly 2040. The exemplary embodiments are shown in FIG. 25 with a cartridge 1000 inserted between the two assemblies. In FIG. 25, the fixed bracket assembly comprises a linear actuator 2014 coupled to a lead nut 2044 (not shown) fixed to a moving bracket assembly described below. The linear actuator uses the lead nut to pull the moving bracket assembly along a linear slide or rail toward the fixed bracket assembly during the clamping action of a cartridge and push the moving bracket assembly away from the fixed bracket assembly during the unclamping action. In another embodiment, the fixed bracket assembly includes one or more sensors to detect when the cartridge is successfully clamped between the fixed bracket assembly 2010 and the moving bracket assembly 2040. As shown in the exemplary instrument illustrated in FIG. 25, the clamping assembly includes sensor 2019 for detecting successful clamping. Upon completion of a testing sequence executed by the instrument, the clamping system may unclamp the cartridge for ejection by the loading assembly.

In another embodiment, the moving bracket assembly is the dynamic component of the clamping subsystem and is configured to move linearly toward the fixed bracket assembly to clamp and contact the cartridge at various locations. In one embodiment, the moving bracket assembly comprises a clamp block. The clamp block may sit along linear slide 2043 corresponding to the fixed bracket assembly. As described above, the linear actuator 2014 is coupled to lead nut 2044 (not shown) on the moving bracket assembly, thus allowing the clamp block to move toward the fixed bracket assembly during the clamping action and move away from the fixed bracket assembly during the unclamping action. In a further implementation, the fixed bracket assembly can comprise one or more sensors for detecting the successful clamping of a cartridge between the fixed bracket assembly and moving bracket assembly.

### 2.2.1.2.1 Establishing Cartridge-Instrument Interfaces

The clamping action of the clamping subsystem establishes one or more cartridge-instrument interfaces to facilitate an instrument testing protocol. Accordingly, one or more portions of the cartridge may physically touch or otherwise interact with the instrument during the testing protocol. For example, an instrument can comprise a mechanism for contacting the cap of the cartridge sample port assembly, ensuring the cap remains closed in embodiments where pressurization of the cartridge is used to advance fluids therethrough. In another embodiment, the clamping subsystem establishes an interface between the instrument and a valve located within the cartridge. In yet another embodiment, the clamping subsystem contains one or more sensors for interacting with the cartridge and/or instrument to determine the establishment of cartridge-instrument interfaces. In further embodiments, the establishment of cartridge-instrument interfaces may impart one or more user visible markings to the cartridge for alerting that the cartridge has been rendered used. For example, the instrument may break a perforated cartridge label, form heat seal marks, or produce any other visual cue on the exterior of the cartridge visible to the user upon ejection and removal from the instrument. While the implementation of establishing one or more cartridge-instrument interfaces varies among instruments and cartridges, a skilled artisan may design other suitable interfaces for facilitating a specific assay or testing protocol. Furthermore, in many embodiments, the plurality of interfaces established by the clamping subsystem enables verification testing to be performed on the cartridge, as further described below.

### 2.2.1.2.2 Maintaining Second Orientation

As described above, a user may load a patient sample into a cartridge in a first orientation and insert the cartridge into the instrument in a second orientation. In such embodiments, the instrument is configured to maintain the cartridge in the second orientation during the testing protocol. In a preferred embodiment, the second orientation is a vertical orientation, as described previously herein. Specifically, the clamping action of the exemplary clamping subsystem provides the infrastructure for orienting and maintaining the cartridge during the testing protocol in the vertical orientation. Maintaining the cartridge in the vertical orientation allows the instrument to leverage the force of gravity to aid fluid movement for processing and liquid handling steps performed.

### 2.3 Verify Label, Sample and System

### 2.3.1 Label Imaging Assembly

As previously described herein, a user may add an identifying mark to a patient label area of the cartridge to provide patient, sample, and/or other testing information to the instrument computer system. Identifying marks allow testing information to be associated with corresponding test results for a given patient. Accordingly, an instrument may comprise an optical subsystem for capturing images of a portion of the cartridge. In one implementation, the optical subsystem is a label imaging assembly configured to capture images of a cartridge patient label area containing an identifying mark prepared by the user. Specifically, capturing the image of the identifiable mark occurs within the instrument without user interaction. A label imaging assembly can comprise at least a label camera and may further comprise a plurality of LEDs, apertures, diffusers, lenses and mirrors. A skilled artisan is capable of designing such an optical assembly for appropriately illuminating a desired area of the cartridge, reshaping such illumination, and minimizing shadows to arrive at an improved image quality.

In one embodiment, a label imaging assembly 2770 (shown in FIG. 24 and FIGS. 29A-B) comprises a camera 2771, LED 2772, aperture 2773 and diffuser 2774. The label imaging assembly 2770 will include at least one, but preferably more than one (e.g., two or three), LEDs 2772 for illuminating a portion of the cartridge, e.g. the patient label area 1040 and sample port assembly, while minimizing shadows cast in the patient label area. The aperture 2773 defines an opening to transmit and reshape illumination by LEDs to reduce off axis light and stray light from affecting the patient label image quality. Once illumination from the LEDs passes through each respective aperture 2773, light travels through diffuser 2774 which generates a more uniform illumination intensity on the patient label and loading module. In one implementation, seen in FIGS. 29A-B, the LEDs may be arranged in an oblique configuration to illuminate the patient label and the adjacent sample port assembly. This arrangement can be advantageous for increasing the contrast of images and improving the overall image quality of the cartridge. Furthermore, the label imaging assembly may further support a plurality of instrument verification tests and provide the user and instrument with relevant testing information.

### 2.3.1.1 Capturing Image of Patient Label Area for Display

The label imaging assembly 2770 is configured to capture an image of a patient label area of the cartridge. The arrangement of the label imaging assembly within the instrument may sufficiently correspond to the cartridge positioning such that the patient label area is within a field of view of the label camera. Such capturing of the patient label area by the imaging assembly allows the instrument to display the image of the patient identifying mark on the instrument graphical user interface and pair the image with test results and/or other information gathered from a cartridge. Accordingly, the user may review the image of the identifying mark in the patient label area for errors and optionally execute a termination command via the graphical user interface if an error is observed.

### 2.3.1.2 Parsing Machine Readable Codes

As previously described herein, the cartridge can contain one or more machine readable codes for providing the instrument and/or user with relevant testing information. In some embodiments, the label imaging assembly captures images of machine readable codes within the field of view of the label camera, enabling the instrument to parse information embedded therein. Embedded information can include, but is not limited to, testing protocol information and cartridge manufacturing information. In one embodiment, the instrument label imaging assembly captures an indication of the type of test to be performed on the cartridge. In a further embodiment, capturing an indication of the type of test to be performed on the cartridge comprises parsing a machine-readable barcode. Such test type further instructs the instrument computer system to perform a specific testing protocol for nucleic acid amplification. By capturing the image with the machine readable barcodes containing embedded information, user interaction with the instrument is unnecessary for launching the correct testing sequence.

Additionally or optionally, the label imaging camera may be configured to read one or more machine readable codes and check against cartridge manufacturing information to generate a determination of an acceptable cartridge for use. In cases where the label imaging assembly captures an image of a machine readable code and the instrument computer system determines the cartridge is not ready for use based on cartridge manufacturing information, the instrument can terminate the testing protocol and eject the cartridge. Such instrument error handling is further described below.

### 2.3.2 Verification Testing

In a preferred embodiment, the instrument performs one or more verification tests without user interaction to further simplify the methods of operating the diagnostic instrument. Performing verification tests without requiring user interaction is beneficial in point of care environments where the level of skill of the operator is unknown. Such verification tests provide an element of reliability by identifying potential issues early into the testing protocol to minimize the potential for an erroneous result. Additionally, by performing verification steps at the beginning of the testing protocol, the operator saves time on a test that may be aborted at a later time during the testing sequence due to a cartridge or instrument failure that could have been detected during this initial stage. Time savings, such as the one described herein, are important in a point of care operating environment where the objective is to test and treat a patient suspected of having an infectious disease before the patient leaves the premises.

The diagnostic instrument may be configured to perform at least one verification test on the instrument, cartridge, sample or any combination thereof after detecting the test type to be performed on the cartridge upon insertion into the instrument. Various cartridge, instrument, and sample verification tests can be implemented during the beginning portion of the testing protocol for confirming cartridge/instrument and sample integrity. Verification tests performed during this portion of the method will vary depending on the specific instrument and cartridge designs implemented, as well as the sample type and amount needed for a proper testing sequence. In some embodiments, the instrument may be configured to display verification testing information to the user and provide the opportunity to cancel a testing sequence during verification testing. Alternatively, in the event that any one of the one or more verification tests fail, the instrument will automatically eject the cartridge (FIG. 18) and the graphical user interface displays a suitable notification regarding cartridge ejection.

FIG. 14 is an exemplary verification test screen, or ‘Reading Cartridge’ screen, displayed for the user while the instrument conducts the one or more verification tests. In some embodiments, the instrument provides the user the option to abort the current testing sequence upon execution of a termination command via user interaction with the graphical user interface. In some embodiments, once all verification tests are satisfactorily completed, the instrument may display information relating to the successful completion of all verification tests. FIG. 15 is an example of a completed cartridge verification screen notifying the user that the instrument testing sequence has begun.

The following descriptions are exemplary verification tests that can be performed by a diagnostic instrument upon initiating a testing sequence.

### 2.3.2.1 Sample Verification Test

Given the low concentrations of target pathogens in some samples, it is advantageous to determine that the user filled a sufficient sample volume in the cartridge sample port assembly. In one implementation, a verification test can comprise confirming a quantity of the sample in the loading chamber of the liquid sample suspected of containing the target pathogen. As described herein, a sample has a volume between 0.2 ml and 5 ml, inclusive, between 0.5 ml and 1.5 ml, inclusive, or is preferably approximately 1 ml. Accordingly, the instrument may be configured to automatically eject the cartridge if an insufficient sample volume is detected during the sample verification test. In such instance, the detection of an insufficient sample volume corresponds to a failed sample verification test.

In one implementation, volume is assessed optically. In such implementation, the cartridge comprises a transparent or translucent window permitting visualization of fluid levels contained within the cartridge. An instrument optical subsystem can provide the hardware for conducting such an optical sample verification test. In one embodiment, the label imaging assembly 2770 may be configured to capture images of at least a portion of a cartridge. This implementation is particularly useful in configurations where the patient label area is adjacent to a sample port assembly and/or within the field of view of the label imaging assembly. Accordingly, the complimentary positioning of such sample window within the field of view of a label camera can allow an instrument to detect and verify that an adequate sample volume is loaded into a cartridge prior to running a diagnostic test. In a preferred implementation, the label imaging assembly is configured to capture an image of the sample port assembly 1100 and detect a mechanism (e.g., a ball disposed within the loading chamber and visible through a sample window) to determine the sample volume. Alternatively, the label imaging assembly may detect the meniscus of the sample fluid through a sample window 1050 provided by the transparent or translucent window to read the volume of the sample. In the case where an insufficient sample volume is detected, the instrument displays to the graphical user interface an error to alert the user that an insufficient sample volume is detected. In some implementations, the error is reported using an error icon, e.g. error icon 1830 characterized by a triangle with an exclamation mark contained therein, to alert the user of the error. As a result, in one implementation, the cartridge is automatically ejected from the instrument if a sample verification test fails and the instrument terminates the remainder of the testing protocol.

### 2.3.2.2 Cartridge Verification Test

To further minimize the number of user interactions required with the cartridge and/or instrument, the instrument may autonomously perform one or more cartridge verification tests for confirming a cartridge is ‘ready for use’. Specifically, the instrument determines the cartridge is ready-for-use by testing whether the cartridge is usable, intact, undamaged, and has not been previously used. In one implementation, a ready for use verification test comprises checking cartridge manufacturing information embedded within one or more machine readable codes, e.g. 1053b, to further determine whether the cartridge is usable for the diagnostic test. Exemplary cartridge manufacturing information may include a cartridge expiration date, lot number, reagent lot numbers, serial numbers, or any other manufacturing information associated with the cartridge. Accordingly, upon capturing of an image of the machine readable code, the instrument parses information contained in the machine readable code to determine whether the cartridge is in an acceptable condition. If the instrument detects an error based on read information, the cartridge fails the verification test and the instrument ejects the cartridge.

Furthermore, a ready-for-use verification test may include checking the physical integrity of one or more portions of the cartridge. In one implementation, checking the physical integrity comprises checking cartridge safety seals and verifying integrity of capsules, blisters, and/or housings configured to store liquids within a cartridge, e.g. determining whether one or more frangible seals are ruptured. In another implementation, the instrument may be configured to test the placement of one or more cartridge components by moving such component from an original position or other indicia of use that may be detected by the instrument. For example, the instrument may detect an incorrect valve position, i.e. a valve position unexpected from an origin position, for a valve contained within the cartridge. Regardless of the type of cartridge verification test performed for determining a usable cartridge, the cartridge is automatically ejected from the instrument, without user interaction, if a cartridge verification test fails and the instrument terminates the remainder of the testing protocol.

### 2.3.2.3 Cartridge-Instrument Interface Verification Test

In yet another implementation, a verification test can comprise completing a cartridge-to-instrument interface test. As described herein, the clamping action of the clamping subsystem establishes one or more interfaces between the cartridge and instrument. The established interfaces may be tested by the instrument to ensure a secure connection was made for proper testing protocol functioning.

In implementations where fluids within the cartridge, e.g. sample, reagents, and/or air, are advanced through the cartridge using a pneumatic force, an instrument pneumatic subsystem can provide one mechanism for performing a cartridge-instrument interface verification test. Generally, pneumatic subsystems are an arrangement of any suitable pneumatic elements, e.g. pumps, valves, regulators, and sensors, configured to generate a pneumatic force to advance fluids throughout the cartridge to different locations for sample processing. In one embodiment, a pneumatic subsystem 2130 is coupled to a cartridge pneumatic interface to deliver said pneumatic force. Such configuration is shown in FIG. 24 and can enable a cartridge-interface verification test to confirm pneumatic integrity by sending an initial pneumatic impulse to the cartridge and successfully reading an expected value. In instances where the instrument does not detect the expected value resulting from the pneumatic impulse, the cartridge-instrument verification test is failed. Accordingly, in yet another implementation, the cartridge is automatically ejected from the instrument if a cartridge-instrument interface verification test fails and the instrument terminates the remainder of the testing protocol.

### 2.3.3 Error Reporting

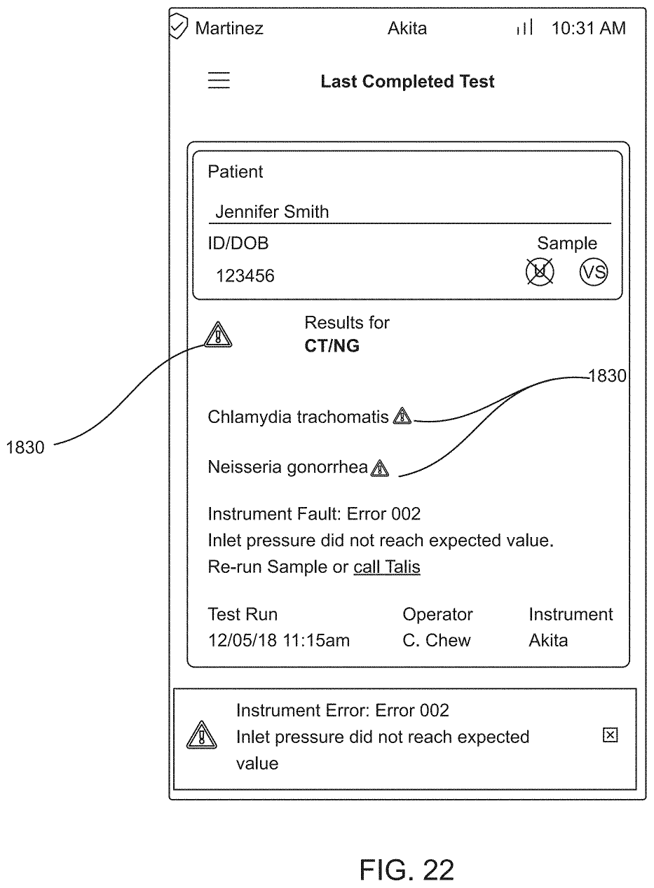

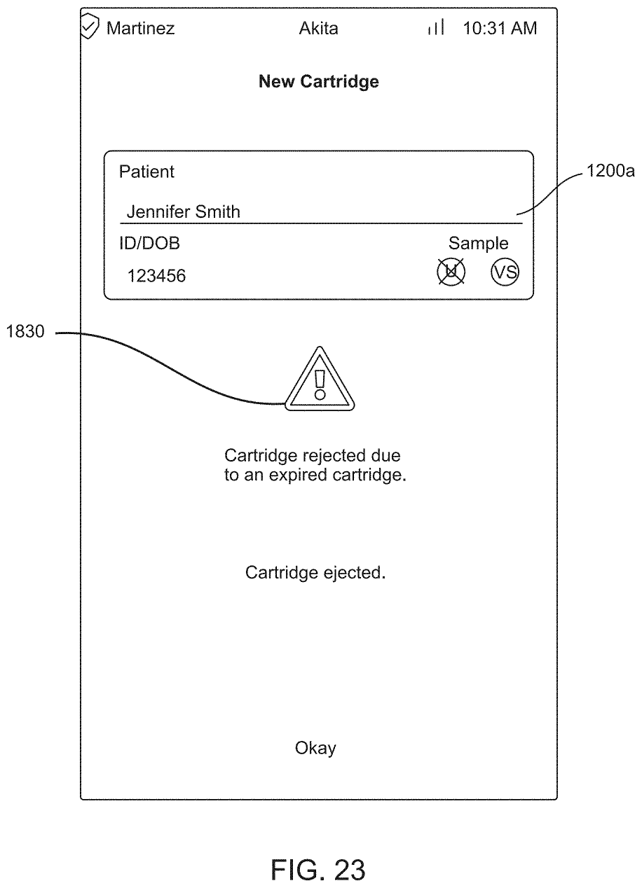

In various implementations, the instrument is configured to display an error screen on the graphical user interface containing information regarding the source of the error. A single indicator, i.e. an icon, indicative of the error may further be displayed to the graphical user interface to alert the user of the error. In one implementation, an error message is displayed resulting from a failed verification test. In another implementation, an error message is displayed resulting from an error detected during the testing protocol. In some embodiments, FIGS. 22 and 23 are exemplary error screens displayed by the instrument. As illustrated in these embodiments, the error icon 1830 is characterized by a triangle with an exclamation mark contained therein. In the embodiment shown in FIG. 22, an instrument error was detected due to an inlet pressure failing to reach an expected value. Accordingly, the instrument may present the user with additional guidance information and ejects the cartridge. As shown in FIG. 21, a failed verification error was detected because the cartridge exceeds a manufacturing expiration date. In such case, the user is presented the option to acknowledge the error, i.e. by interacting with the GUI portion to select ‘Okay,’ and the cartridge is subsequently ejected. Additional details of cartridge, instrument and sample verification along with error generation and error handing are provided in U.S. Non-Provisional patent application Ser. No. 16/655,007, entitled “Diagnostic System” filed Oct. 16, 2019 and U.S. Non-Provisional patent application Ser. No. 16/655,028, entitled “Diagnostic System” filed Oct. 16, 2019, each of which is incorporated herein by reference for all purposes.

### 2.4 Display Patient Identifying Mark

As previously described herein, a user can add an identifying mark to a patient label area of the cartridge to provide patient, sample, and/or other testing information to the instrument computer system. Accordingly, in one embodiment, the instrument optical subsystem is a label imaging assembly, e.g. the one shown in FIGS. 24 and 29A-B, configured to capture an image of the patient label area containing an identifying mark prepared by the user. In some embodiments, the instrument is configured to display an image, e.g. an image taken from the label imaging camera, of the identifying mark for review by the user of the instrument. In another embodiment, the instrument displays an image of the identifying mark and the test type to be performed on the cartridge. Specifically, the test type may be indicated by text. In some embodiments, the identifying mark in the patient label area is displayed for a predetermined time less than 2 minutes, less than one minute, less than 30 seconds, or less than 10 seconds. Alternatively, the predetermined time may be any other time period inherent to the instrument default settings or can be selected by a system administrator. In a preferred embodiment, the predetermined time is 10 seconds. In further embodiments, the instrument permits the user to manually halt, i.e. cancel or terminate, testing within the predetermined time period of displaying the identifying mark on the graphical user interface. By way of example, consider the prominent “Abort” option along the lower display portion of the GUI illustrated in FIGS. 13 and 14. Furthermore, permitting the user to manually cancel the testing protocol can comprise providing the user with the option to select a termination command displayed on the graphical user interface. In embodiments where the graphical user interface is a touch screen, canceling a testing protocol may be accomplished by interaction with a readily identifiable portion of the GUI display. Upon interaction by the user within the predetermined time, the instrument ejects the cartridge in response to receiving the termination command.

Alternatively or additionally, the instrument may permit the user to begin a testing protocol prior to the expiration of the predetermined period of displaying the identifying mark on the graphical user interface. This embodiment allows the instrument to initiate the testing protocol either before the elapse of the predetermined period for receiving a termination command or the instrument will initiate the testing protocol after the elapse of the predetermined period. FIG. 13 is an exemplary screen displayed to the user for observing the patient identifying mark and test type to be performed. In the illustrated embodiment, the instrument prompts the user to verify the patient information, i.e. name, date of birth, sample type, and test type, is correct. Additionally, the instrument displays the user the option to terminate the current testing protocol by selecting the ‘Abort’ portion of the GUI or initiate the current testing protocol by selecting the ‘Start Now’ portion of the GUI before the time period of 10 seconds elapses.

### 2.5 Initiate and Complete Testing Protocol

Without so limiting the invention, in many embodiments, the instrument is configured to perform a nucleic acid amplification test/assay with a matched integrated diagnostic cartridge using a variety of subsystems and assemblies. The instrument interacts with the cartridge through established cartridge-instrument interfaces to perform sample preparation, target nucleic acid amplification, and signal detection. In particular, the details of an appropriate instrument configured to perform a nucleic acid amplification test/assay with a matched integrated diagnostic cartridge is further described in detail in U.S. Non-Provisional patent application Ser. No. 16/655,007, entitled “Diagnostic System” filed Oct. 16, 2019 and U.S. Non-Provisional patent application Ser. No. 16/655,028, entitled “Diagnostic System” filed Oct. 16, 2019, each of which is incorporated herein by reference for all purposes. Accordingly, those of ordinary skill in integrated cartridges and point of care instruments may select and implement any appropriate automation sequences based on the details provided herein in furtherance of one or more of the recommendations, guidelines and requirements described above for achieving the objectives of CLIA waived testing. As a result, various embodiments of automated workflows and minimum user interaction workflows will result based on implementations in specific instrumentation to accomplish sample preparation, target nucleic acid amplification and signal detection for desired testing sequences and targets.

### 2.5.1 Potential Testing Protocols

In some embodiments, appropriate testing protocols for nucleic acid amplification to determine to the presence, absence, or quantity of a target pathogen include, but are not limited to, methods such as polymerase chain reaction (PCR), loop-mediated isothermal amplification (LAMP), strand displacement amplification (SDA), recombinase polymerase amplification (RPA), helicase dependent amplification (HAD), multiple displacement amplification (MDA), rolling circle amplification (RCA), and nucleic acid sequence-based amplification (NASBA).

To perform such nucleic acid amplification, a diagnostic cartridge is used in conjunction with a diagnostic instrument according to the methods described herein for producing an indication of a presence, absence, or quantity of a target pathogen. The diagnostic cartridge can comprise a plurality of modules for housing sample preparation, amplification, and signal detection steps. Accordingly, such plurality of modules may further be configured to interact with respective instrument assemblies and subsystems for performing the testing protocol. Such cartridge modules may include one or more modules relating to loading a sample into the cartridge, lysing the patient sample, purifying the sample, and amplifying the sample for analysis.

### 2.5.2 Sample Preparation

Generally, sample preparation refers to the treatment of a sample prior to analyzing said sample, e.g. to detect the presence of nucleic acids indicative of target pathogens. Factors such as sample type and analyte type affect the selection of specific sample preparation techniques and procedures. In many implementations, the instrument facilitates cell lysis to break or disintegrate the outer boundary or cell membrane to release inter-cellular materials such as nucleic acids (DNA, RNA), protein or organelles from a cell by magnetically mixing the sample with preparation solution, e.g. a cell lysing agent and/or buffer. Lysis resulting in the release of nucleic acids can be achieved by chemical, enzymatic, physical and/or mechanical interventions. After lysing, the instrument advances the sample though a filter to remove cell debris and material.

Subsequently, the instrument purifies the filtered sample by passing said sample through a capture matrix to extract and isolate nucleic acids contained therein. A washing step is performed to remove contaminants from the capture matrix to minimize the presence of inhibitors in the final reaction. An elution step reverses the binding of nucleic acid to the capture matrix and releases said nucleic acid, resulting in a purified sample. Such purified sample, resulting from sample preparation steps, may then be advanced to other modules, containing at least one reaction chamber or well, for amplification and detection of suspected target pathogens.

As previously described herein, the instrument is configured to be used with a matched diagnostic cartridge for performing the nucleic acid amplification test. The cartridge may contain appropriate modules corresponding to instrument assemblies and subsystems for performing a testing protocol to arrive at a diagnostic test result. As described herein, the cartridge contains a loading module to receive a sample, minimize the spilling of the sample, and prepare the sample for lysis. In one implementation, the loading module is a sample port assembly comprising at least a sample port for permitting limited access to the interior of the cartridge to load a patient sample. In further embodiments, the sample port assembly may further comprise a loading chamber for storing the patient sample until said sample is advanced to other locations within the cartridge for sample processing and a cap for sealing the sample port assembly. Preferably, the cap is configured to prevent the re-opening after a sample is added and said lid is closed. The sample loading assembly 1100 is viewed in FIG. 3.

In various implementations, the cartridge contains a lysis module for lysing the loaded patient sample. In some implementations, the lysis module comprises one or more structures, i.e. at least one lysing chamber, configured for exposing a sample to a preparation solution, e.g. a cell lysing agent and/or buffer, to produce a lysed sample. Producing the prepared biological sample can require structures, such as a mixing chamber, for exposing the preparation solution to the patient sample, wherein such exposure results in the rupturing of cell walls or cell membranes to release inter-cellular materials such as nucleic acids (DNA, RNA), protein or organelles from a cell. In further implementations, structures within the lysing module can contain additional structures therein for performing mechanical lysis. Such mechanical lysis elements include but are not limited to stir bars, ceramic beads, glass beads, and steel beads. In the exemplary cartridge shown in FIG. 3, the area of the cartridge allocated for the lysing module is shown by an outer dashed circle encompassing lysing chamber 1371. In another embodiment, the lysis module includes a filter for removing the majority of cell debris from the lysed sample. Filtration, in many cases, is advantageous for removing larger cellular material for preventing clogging during downstream purification. Filters can be any of a size exclusion filter, a depth filter, membrane filter, plasma filter, an ion-exclusion filter, a magnetic filter, or an affinity filter.

In another implementation, the cartridge contains a purification module for extracting and/or purifying nucleic acids from the lysed patient sample. In some implementations, such nucleic acid purification is performed by one or more cartridge structures, e.g. a capture matrix or porous solid support, for binding nucleic acids and removing contaminants and other cellular debris from the lysed sample. In certain implementations, the capture matrix has an affinity for nucleic acids, such that the nucleic acids are captured by the capture matrix while proteins, lipids, polysaccharides, and other cell debris that can inhibit nucleic acid amplification pass through the matrix. In some implementations, after capturing the nucleic acid, a wash solution is passed through the capture matrix to further remove contaminants. Captured nucleic acid is then released from the matrix with an elution buffer to generate an enriched nucleic acid for amplification. Accordingly, a skilled artisan capable of selecting an appropriate capture matrix material based on considerations such as the chemical nature of the affinity ligand pair and how readily the matrix can be adapted for the desired specific binding.

In a further implementation, the purification module can include additional structures, e.g. chambers, housings, or other any other vessel, formed therein configured to store on-board liquid and dried-down reagents to be used for conducting the sample preparation, nucleic acid amplification and/or liquid waste generated during sample preparation. In another implementation, the purification module further comprises a rotary valve for direction fluids, such as the patient sample, reagents, and air, to various locations within the cartridge for processing. In the exemplary cartridge shown in FIG. 3, the area of the cartridge allocated for the purification module is shown by a circle encompassing area 1400.

### 2.5.3 Amplification and Signal Detection

In some embodiments, the amplification reaction is a real time reaction, such that the instrument monitors the amplification of target nucleic acids during the reaction, i.e. in real time. In some embodiments, the amplification of target nucleic acids is detected using fluorescent labels. In such embodiments, the monitoring of the reaction wells for a fluorescent signal during the amplification is provided by at least one optical subsystem. The optical subsystem can be configured to capture images of a reaction area containing a plurality reaction wells during the testing protocol to determine the indication of a presence, an absence, or a quantity of a target pathogen.

Accordingly, in some embodiments, the cartridge used in conjunction with the diagnostic instrument contains a corresponding amplification module for generating and detecting a signal indicative of the presence of a target pathogen in the sample. In some implementations, the amplification module may include a reaction area comprising a plurality of structures, e.g. reaction areas, chambers, or wells, for conducting the nucleic acid amplification reaction. In some implementations, the purified nucleic acids may be combined with one or more amplification reagents within the reaction areas. In many implementations the one or more amplification reagents comprise a primer or primer set. The primer set can be specific to a first nucleic acid sequence present in one of the one or more target pathogens. In alternative implementations, the cartridge may be configured to provide a plurality of primer sets for detecting a second, third, fourth, or any number of target pathogens within the reaction areas. As illustrated in the exemplary cartridge shown in FIGS. 2 and 3, the reaction area 1600 is positioned at a distal end of the cartridge opposite to the patient label area 1040 and sample port assembly 1100. In such implementation, the positioning of reaction area 1600 corresponds to a second optical subsystem, differing from the label imaging assembly 2770, for monitoring the amplification of target nucleic acids during the reaction.

### 2.6 Display Test Result

As previously described above, limited user interaction with the instrument is required after the instrument initiates a testing protocol to determine presence or absence of a target pathogen. Upon the completion of a testing protocol, a user observes an image of the identifying mark and an indication of a presence, an absence, or a quantity of a target pathogen in the sample on the graphical user interface. In various aspects of the invention, the instrument automatically displays on the graphical user interface the indication of a presence, an absence, or a quantity of the target pathogen resulting from the testing protocol. In some embodiments, the indication, i.e. test result, is displayed automatically without any user interaction with the graphical user interface or the instrument. In other embodiments, the indication is displayed after user interaction with the graphical user interface. FIG. 17 is an example of a ‘Latest Completed Test’ result screen for displaying the indication of the presence or absence of the target pathogen. As previously described above, the instrument displays a positive icon 1800 to indicate the positive presence of CT in the patient sample and a negative icon 1805 to indicate the absence of NG in said sample. Additionally, the instrument displays a “Results Summary’ icon for notifying the user that the instrument detected at least one target pathogen. The illustrated embodiment in FIG. 17 depicts a positive summary icon 1810 for representing the presence of at least one positively detected target pathogen from the two or more test results.

### 2.6.1 Instrument Privacy

In many embodiments, the instrument includes one or more security features to ensure privacy and the protection of patient health information. In one embodiment, the instrument displays the indication of the presence, the absence, or quantity of a target pathogen for a predetermined time period of 5 minutes or less, 2 minutes or less, 1 minute or less, or 30 seconds or less. In another embodiment, the instrument prevents the display of individual test results on the graphical user interface after the predetermined time period. Specifically, in some embodiments, the instrument displays an idle screen or a start screen. However, in embodiments where the instrument displays an idle screen, the instrument can allow the display of individual test results after entering a security code using the graphical user interface or performing any non-contact or near field or other security and/or user identification function.

### 2.7 Eject Cartridge

As described previously described herein, the instrument may be configured to eject the cartridge during normal instrument operation upon the completion of a testing sequence, as a result of a user initiated termination command, or as a result of an error. In one embodiment, the instrument loading assembly 2230 provides the mechanism for ejecting a cartridge, such that after an unclamping action is performed by the clamping subsystem, a spring 2235 in the loading assembly relaxes toward equilibrium position to eject the cartridge. In some embodiments, the instrument ejects the cartridge while displaying the test result or error message. In other embodiments, the instrument ejects the cartridge after displaying the test result or error message. An exterior view of the instrument ejecting a cartridge is viewed in FIG. 18.

### 2.8 Computer Controller System

An instrument computer controller system operates the variety of instrument assemblies and subsystems for generating reliable diagnostic results while requiring minimal user interaction. The sample processing, amplification, and/or detection steps can be automated using an appropriate computer controller system to facilitate the ease of use for the user/operator by minimizing the number of steps performed by the user. After inserting a cartridge, the computer controller system executes a sequence of steps to operate the instrument assemblies and subsystems. The total automation of such molecular testing sequence allows a lay or untrained user to perform a diagnostic test with ease while simultaneously minimizing the risk of an erroneous result due to human error. Still further, implementation of various automated computer instructions according to the workflow embodiments detailed herein may be used to achieve many of the recommendations, guidelines and requirements detailed above with little or no user interaction.

FIG. 30 represents a schematic view of a representative computer control system for use with sub-systems and components of a diagnostic instrument described herein. Generally, the instrument computer control system includes instructions in computer readable code used to coordinate the synchronous performance of the one or more of the operations performed by a specific instrument—cartridge—testing sequence related to receiving, handling, processing and analyzing a suspected sample in a cartridge. Additional details of the various steps performed related to receiving, handling, processing and analyzing a suspected sample in a cartridge will vary based on a number of instrument and cartridge design factors. The computer system may comprise an exemplary client or server computer system. Computer system includes a number of communication channels or busses for communicating control signals, sensor information, or other information from a component or system within the instrument to a processor. These various communication pathways are indicated by the lines connecting each of the various components, systems and subsystems. The host processor 2900 is used for processing information and generating signals according to one or a number of programmed control sequences. Processor 2900 may be any suitable computer controller, processor with co-processor, microprocessor or suitable combination thereof.

Additionally or optionally, the instrument computer control system may include one or more of a random access memory (RAM), or other dynamic storage device (referred to as main memory) coupled to bus for storing information and instructions to be executed by processor. Main memory also may be used for storing temporary variables or other intermediate information during execution of instructions by processor.

Instrument computer system also includes a read only memory (ROM) and/or other static storage device coupled to bus for storing static information and instructions for processor, and a data storage device, such as a magnetic disk or optical disk and its corresponding disk drive. Data storage device is coupled to bus for storing information and instructions.

With reference to FIG. 30, the host processor 2900 is in communication with a communications module 2905 which includes a cellular antenna 2800 located in the front panel 2073 of the instrument 2000 along with associated firmware and software. Additionally, the host processor 2900 is in communication with a USB and Ethernet port 2903 as well as any other external communication port. There is access provided to data storage including encrypted data 2901 along with calibration, firmware upgrade and test results data. There is also provided appropriate storage for de-identified patient results data.

The host processor 2900 is also in communication with a display or graphical user interface 2902 such as the one on the instrument front panel 2073. The host processor 2900 can send display data to the display 2902, which can then output the display data for the user to view. The display data may be presented in a simple manner to include status or operational information based off of a process the instrument, a cartridge, as well as test data including the test results and error data to indicate whether any operational errors have occurred during the testing. See, for example, the variety of information provided in exemplary GUI displays shown in FIGS. 13-15 as well as FIGS. 20-23. In some embodiments, the GUI display 2902 can be touch sensitive so that the user can confirm control information for initiating or canceling operation of a test as in FIGS. 11 and 13. Still further, in accordance with various embodiments herein, the GUI display 2902 presents other data, such as an image of an identifying mark (FIG. 16) as well as one or a series of test results (FIGS. 17, 19A and 19B) in an easy to read format. The GUI display 2902 may be, but is not limited to, an OLED or LCD display with touch screen capabilities and easy to identify actions as shown in the various GUI display examples. Still further, host processor 2900, through executing certain programs such as a graphical user interface (GUI) engine, generates a user interface which is then shown on the GUI display 2902. The GUI engine provides data according to a certain layout for each user interface and also receives data input or control inputs from the user. The GUI then uses the inputs from the user to change the data that is shown on the current user interface, or changes the operation of instrument or initiates automatic processes as described herein. It is to be appreciated that the arrangement of the GUI display as well as the types of prompts or interactions with the user may be modified according to the specific instrument, cartridge and testing sequence that is implementing the inventive workflows described herein.

Still further, the computer system and instrument may include any of a wide variety of near field communication, non-contact, RFID or smart device communication capabilities for instrument access, user identification or security protocols. By way of example, a form of non-contact or near field identification may be used instead of the security code screen shown in FIG. 10. Additionally or optionally, a security, access or identification token associated with a user may be introduced into the instrument load slot and read by the label imaging system described in FIGS. 29A and 29B. Additionally or optionally, the near field or non-contact capability may be provided behind or adjacent to the GUI display such that a user may hold near or pass a security, access or identification token in proximity to the GUI display or according to instructions provided on the GUI display.

The host processor 2900 is also in communication with various instrument application software 2904. This software and firmware corresponds, by way of example, to particular testing routines to be implemented by the diagnostic instrument 2000 based on the type of sample/integrated diagnostic cartridge 1000 that is loaded into and detected by the instrument 2000. Additionally, the instrument software and firmware 2904 includes computer readable instructions for an instrument operating system along with the various appropriate computer drivers for instrument components. The host processor 2900 is also configured to access and execute the camera operation and imaging firmware 2915 responsible for executing the specific imaging routines performed by the label imaging camera 2771 and the reaction chemistry or assay chamber camera or other appropriate imaging systems.

Advantageously, the instrument computer system may include a host processor and a co-processor 2900 in coordinated operation. In one configuration, the host processor 2900 includes instrument operating system and device drivers, specific instrument application software and firmware 2915 for operation of the label camera 2771 and any reaction well camera, as needed. A second processor may be configured as a slave processor to handle other commands such as the operation of various motors and actuator in the diagnostic instrument 2000. Additionally, the co-processor would be responsible for prioritization and execution of various control signals throughout the various instrument subsystems. The instrument computer system memory or computer readable storage may include stored or accessible computer records of various test methods, scripts, parameters, completed records storage, access records, security/access protocols, instrument calibration readings and results based on specific operations performed by the instrument 2000 for a specific cartridge diagnostic test or sample type.

In general, an instrument computer system includes the appropriate functional subsystems adapted and configured to correspond to the steps performed in a wide variety of functions corresponding to a desired instrument, cartridge, sample types and preprogrammed functions or testing sequences. FIG. 30 includes only those subsystems involved in the exemplary workflow embodiments described herein. The optical cartridge label subsystem 2910 is used in the operation and control of components illustrated and described in FIGS. 29A and 29B. The loading cartridge subsystem 2920 is used the operation of components in FIGS. 24, 25, 26A, 26B to provide stabilization of a cartridge within the instrument as well as automatic cartridge ejection. The cartridge seal rupturing subsystem 2930, the pneumatic-interface subsystem 2960, the valve drive subsystem 2940 are used in the coordinated operation of components in FIGS. 24 and 25 and may be employed in the performance of a number of instrument and cartridge validation or confirmation processes as described herein. Other additional subsystems not shown in FIG. 30 are also included to perform the various other functions according to the specific instrument configuration, integrated cartridge design and chemistry implementation. Other subsystems under control of an instrument computer controller include, for example, an optical reaction well subsystem, a thermal subsystem, a lysing drive subsystem, and a rehydration mixing subsystem.

Additional alternative computing environments and modifications to both user experience and user interaction are possible and within the scope of the various embodiments described herein, The instrument computer control system may further be coupled to a display device, such as a liquid crystal display (LCD) including touch screen or other functionality by direct connection or wirelessly. The display is also coupled to bus for displaying information to an instrument user. An alphanumeric input device, including alphanumeric and other keys, may also be provided via the touch display or coupled to bus for communicating information and command selections to processor. An additional user input device is cursor control, such as a mouse, trackball, trackpad, stylus, or cursor direction keys, voice or touch controllers coupled to bus for communicating direction information and command selections to processor, and/or for controlling cursor movement on display.

Note that any or all of the components of system and associated hardware may be used, altered or modified in a particular instrument—cartridge—testing configuration. However, it can be appreciated that other configurations of the instrument, cartridge, and computer system may include some or all or different subsystems, additional or different subsystems, components or sensors. Certain variations of system may include peripherals or components not described in these various exemplary subsystems but would be understood as included in a specific instrument—cartridge—testing configuration. Additional such components and subsystems may be included and configured to receive different types of user input, such as audible input, or a touch sensor such as a touch screen or near field communications.

Certain embodiments may be implemented as a computer program product that may include instructions stored on a machine-readable medium. These instructions may be used to program a general-purpose or special-purpose processor to perform the described operations. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; electrical, optical, acoustical, or other form of propagated signal (e.g., carrier waves, infrared signals, digital signals, etc.); or another type of medium suitable for storing electronic instructions. The label imaging camera firmware or the optical cartridge label subsystem may be adapted and configured to recognize machine readable markings as part of a cartridge verification protocol as well as to aid in the identification of a particular sample type and/or diagnostic testing routine to be performed with that sample/cartridge.

Additionally, some embodiments may be practiced in distributed computing environments where the machine-readable medium is stored on and/or executed by more than one computer system. In addition, the information transferred between computer systems may either be pulled or pushed across the communication medium connecting the computer systems.

The digital processing device(s) described herein may include one or more general-purpose processing devices such as a microprocessor or central processing unit, a controller, or the like. Alternatively, the digital processing device may include one or more special-purpose processing devices such as a digital signal processor (DSP), an application specific integrated circuit (ASIC), a field programmable gate array (FPGA), or the like. In an alternative embodiment, for example, the digital processing device may be a network processor having multiple processors including a core unit and multiple micro engines. Additionally, the digital processing device may include any combination of general-purpose processing device(s) and special-purpose processing device(s).

### Examples

By way of introduction, the inventive point of care workflows are described using an exemplary point of care diagnostic system shown in FIGS. 1, 4, and 5. This system is configured as an in vitro diagnostic device that is designed to detect nucleic acids of target pathogens in collected body fluid specimens. The instrument is intended to be operated in a point-of-care, i.e., near-patient, environment, such as a physician's practice, health clinic, or within a patient treatment room in a hospital. The system is intended to be simple enough to operate and pose such an insignificant risk of an erroneous result that it can be suitable for a Clinical Laboratory Improvement Amendments (CLIA) Waiver categorization. Consequently, the system does not require highly trained personnel to operate and perform the molecular diagnostic test.

In many aspects, the instrument is configured to perform a qualitative in vitro real-time Nucleic Acid Amplification Test for the automated detection of differentiation of nucleic acids using a matched integrated diagnostic cartridge. In one implementation, the instrument performs a test for the detection of nucleic acids from Chlamydia trachomatis (CT) and/or Neisseria gonorrhoeae (NG) to aid in the diagnosis of chlamydial and gonorrheal urogenital disease. Specifically, the nucleic acid amplification uses a qualitative loop mediated isothermal amplification (LAMP) assay for the detection and determination of nucleic acids from CT/NG. The assay may be used to test the aforementioned specimens from asymptomatic and symptomatic individuals: female and male urine, patient-collected vaginal swabs (collected in a clinical setting) and clinician collected vaginal swabs.

The Talis One CT/NG Assay is performed on the Talis One Instrument with a single-use Talis One CT/NG Assay Kit which includes, at least, a disposable Talis One CT/NG Assay Cartridge and a sample transfer pipette. The instrument automates and integrates sample purification, nucleic acid amplification, and detection of the target nucleic acid sequences in urogenital samples using real-time loop mediated isothermal amplification (LAMP) on the Talis One CT/NG Assay Cartridge. Appropriate mechanical, pneumatic, thermal, and optical subsystems are incorporated into the instrument to interface with the cartridge. Correspondingly, the test cartridge, used in conjunction with the instrument, is designed to receive a sample, minimize the spilling of the sample and serve as a contained vessel for conducting the assay on the instrument. All liquids and reagents remain isolated in the cartridge and do not contact the Instrument.

### 3.1 Example 1: Minimal User Interaction

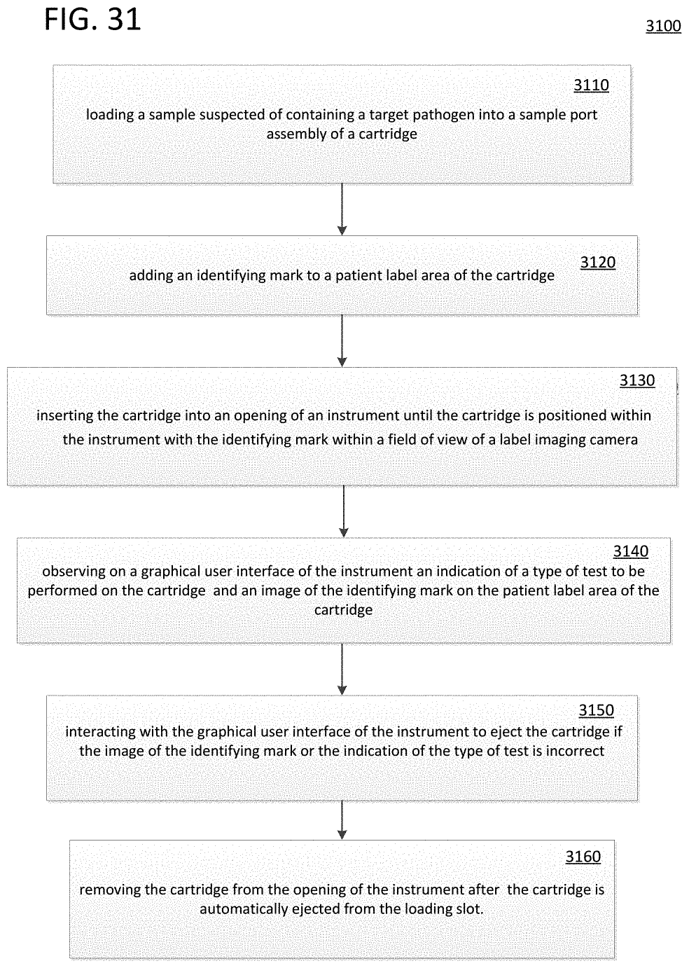

FIG. 31 is a flow chart of an exemplary method 3100 of operating an instrument for testing a sample suspected of containing a target pathogen. Embodiments of this illustrative workflow aid in accomplishing one or more of the recommendations, guidelines and requirements above such as ease of use and patient sample identification.

First, at step 3110, there is a step of loading the sample suspected of containing the target pathogen into a sample port assembly of a cartridge.

Next, at step 3120, there is the step of adding an identifying mark to a patient label area of the cartridge.

Next, at step 3130, there is the step of inserting the cartridge into an opening of the instrument until the cartridge is positioned within the instrument with the identifying mark within a field of view of a label imaging camera.

Next, at step 3140, there is a step of observing on a graphical user interface of the instrument an indication of a type of test to be performed on the cartridge and an image of the identifying mark on the patient label area of the cartridge.

Next, at step 3150, there is a step of interacting with the graphical user interface of the instrument to eject the cartridge if the image of the identifying mark or the indication of the type of test is incorrect.

Next, at step 3160, there is a step of removing the cartridge from the opening of the instrument after the cartridge is automatically ejected from the opening.

In one alternative embodiment, the method may also include a step of observing an error message on the graphical user interface before or during the removing step. One exemplary message is provided in the GUI display of FIG. 22 indicating either an instrument failure or an error in cartridge integrity. An additional error message for an expired cartridge is shown in the GUI display of FIG. 23. In either of these or in similar cases, such an unauthorized, suspected fraudulent or cartridge recalled by the manufacturer, the error prone cartridge is automatically ejected by the instrument as shown in FIG. 18 without interaction with or action by the operator.

In one alternative embodiment which greatly aids in accomplishing correct pairing of patient sample to results as well as patient identification is shown in the exemplary GUI display of FIG. 17. In this aspect, as is clearly shown in FIG. 17, the method accomplishes a step of observing an image of the identifying mark and an indication of a presence, an absence or a quantity of the target pathogen in the sample on the graphical user interface before or during the removing step.

In one alternative embodiment, the method may also include a step of automatically initiating a testing protocol when a predetermined time period has elapsed after completing the inserting the cartridge step. An illustrative count down timer is shown in the exemplary GUI display of FIG. 16. Automated initiation of testing frees an operator from the requirement to monitor or tend to additional steps for operation of an instrument. Advantageously, in order to enable additional time savings, the predetermined time period may be selected is less than 2 minutes, less than one minute, less than 30 seconds, or less than 10 seconds. Optionally, to save even more time, there may be a step of interacting with the graphical user interface to initiate a testing protocol after the observing step has been performed. This action may be accomplished by simply interacting with the “Start Now” shown in the lower portion of the GUI display as shown in FIG. 14.

Advantageously, in order to meet the recommendations, guidelines and requirements of sample custody and identification, the step of adding an identifying mark may include simply affixing a printed label or a printed machine readable label to the patient label area. Additionally or optionally, the patient label area is adjacent the sample port assembly. Still further, the step of adding an identifying mark step may also include a step of handwriting sample identifying information in the patient label area. Adding an identifying mark is shown and described with regard to FIGS. 4A and 4B. These views along with those of FIGS. 2, 5 and 6 also illustrate the proximity of the patent label area to both a sample port assembly and an indicator of sample quantity.

In one alternative embodiment illustrated in FIG. 10, a step includes touching the graphical user interface to enter a security code after performing the observing step. In additional alternatives, there may also be a step of observing on the graphical user interface a progress timer of the testing protocol as in FIG. 16 or a listing of one or more previously tested protocol results as in FIGS. 17, 19A and 19B.

Additionally, the method may ensure completion of the guidelines and recommendations by automatically performing certain instrument functions after performing the inserting the cartridge step. In one example, the instrument initiates at least one cartridge verification test without any user interaction with the instrument after performing the inserting the cartridge step. In other aspects, the instrument may also automatically perform other instrument verification or cartridge-instrument verification or sample verification steps specific to an instrument or cartridge implementation in furtherance of the guidelines and requirements related to following a manufacture's indications for use or testing protocols.

In still other alternative embodiments, the method may also include a step of observing on the graphical user interface of the instrument the identifying mark without touching the graphical user interface of the instrument or performing any other user interaction to contact the instrument while or prior to performing the removing the cartridge step. In additional variations, after the inserting a cartridge step is performed, the instrument will automatically perform a nucleic acid amplification process to produce a result that contains an indication of a presence, an absence or a quantity of the target pathogen in the sample suspected of containing the target pathogen. Importantly and advantageously, this step is performed without touching a graphical user interface of the instrument or otherwise interacting with the instrument after the inserting step. In some embodiments, the steps performed are part of a sequence specific detection process adapted to the target pathogen and the cartridge-instrument design factors. Implementation of such automatic steps which require no interaction with the instrument also play an important role of implementing the guidelines and requirements detailed above.

In still other alternative embodiments, the instrument workflow may include a step of observing an image, an icon or a glyph on the graphical user interface indicating a result of a testing protocol performed during, after or before the removing step. Exemplary displays which illustrate the ready comprehension of results are illustrated in FIGS. 19A and 19B. Depending upon a number of factors related to sample type, cartridge design and instrument capabilities the length of time to performance of the observing step may vary. In some exemplary implementations, the step of observing the image, the icon of the glyph on the graphical user display is performed less than 60 minutes, less than 25 min, less than 20 min, less than 15 min, or less than 10 min after inserting a cartridge into the instrument.

### 3.2 Example 2: Single Instrument Interaction to View Single Indicator of Result

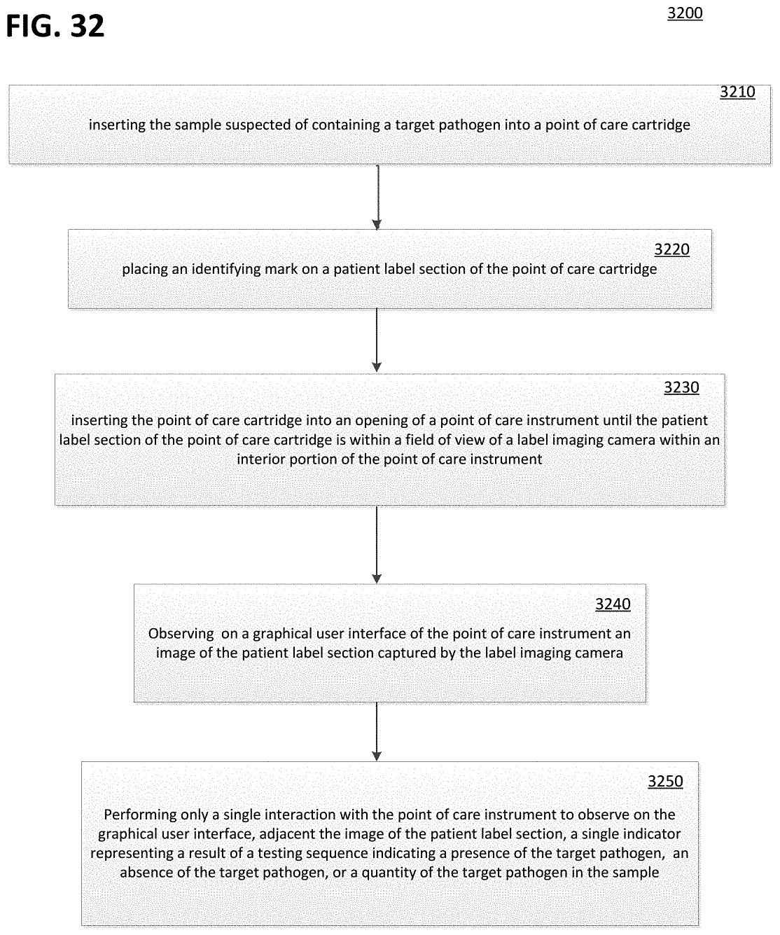

FIG. 32 is a flow chart of an exemplary method 3200 of testing a sample suspected of containing a target pathogen. Embodiments of this illustrative workflow aid in accomplishing one or more of the recommendations, guidelines and requirements above. For example, an advantage of this method is the presentation of a single indication of a result in the same field of view in the GUI as the user selected identifying mark.

The method 3200 of testing a sample suspected of containing a target pathogen includes a step 3210 of inserting the sample suspected of containing the target pathogen into a point of care cartridge. (See FIGS. 5 and 6).

Next, at step 3220, there is a step of placing an identifying mark on a patient label section of the point of care cartridge. FIGS. 4A and 4B illustrate the latitude available to a user in terms of patient identification on the point of use cartridge.

Next, at step 3230, there is a step of inserting the point of care cartridge into an opening of a point of care instrument until the patient label section of the point of care cartridge is within a field of view of a label imaging camera within an interior portion of the point of care instrument.

Next, at step 3240, there is a step of observing on a graphical user interface of the point of care instrument an image of the patient label section captured by the label imaging camera. So long as the patient label is not blank, a user has a wide range of options for use as an identifying mark since that very mark is automatically provided to the GUI (see FIG. 13) based on the operation of the label imaging system (see FIGS. 29A, 29B).

Next, at step 3250, there is a step of performing only a single interaction with the point of care instrument to observe on the graphical user interface, adjacent the image of the patient label section, a single indicator representing a result of a testing sequence indicating a presence of the target pathogen, an absence of the target pathogen, or a quantity of the target pathogen in the sample.

In an additional aspect of the method, the placing step may also include handwriting on the patient label section to identify the sample. Additionally, the placing step may also include affixing a printed label in the patient label section to identify the sample. Additionally, the placing step may also include marking a pre-printed box, circle, geometric shape, or area in the patient label section indicating a sample type contained in the point of care cartridge.

In further variations, there may also be a step of initiating a point of care instrument testing sequence on the sample in the point of care cartridge without user interaction after a predetermined time delay or immediately upon user interaction with a portion of the graphical user interface.

In still additional variations, the method may also include a variety of alternative ways of displaying results. By way of non-limiting examples, there may be a presentation of information on the graphical user interface of: (1) a single indicator of positive/negative for a test result; (2) an optional ‘drop down’ list for individual test results; (3) a graphical indicator (color and icon); (4) a timeout of results display for security; or (5) access list of past results.

In an additional aspect of the method, a time delay of less than 15 minutes separates the observing step from the performing step. In other aspects, based on cartridge design and sample type, the time delay may be more than 15 minutes, between 15 to 20 minutes or between 20 minutes to 30 minutes.

In an additional aspect of the method, the step of performing a single interaction further comprising entering a security code into the graphical user interface to permit interaction with the point of care instrument. In another alternative, after the inserting step the point of care cartridge is substantially within the interior of the point of care instrument. In still another alternative, the performing only a single interaction step is undertaken after observing that the point of care cartridge is ejected from the point of care instrument.

In an additional aspect of the method the single indicator represents a positive test result or a negative test result. In still another variation, the single indicator for the positive test result appears in red in the GUI. Still further, the single indicator for the negative test result appears in green in the GUI. In another aspect the single indicator is an image, icon, or glyph. In another alternative, the single indicator comprises a number of text characters. It is believed that these additional advantageous features will also aid a user or operator in meeting the guidelines, requirements and objectives above given the ease with which a single indicator may be used to provide an accurate summary of many tests performed on a cartridge but with which no further action is required.

In an additional aspect of the method, the single indicator representing a result includes an image, an icon, or a glyph for a presence of the pathogen or an absence of the pathogen. In additional alternatives, the single indicator represents a result for two or more tests performed on the point of care cartridge. In another aspect, the single indicator represents a negative presence of all target pathogens from the two or more tests or a single indicator represents a positive presence of at least one target pathogen from the two or more tests.

In an additional aspect of the method there is also a step of interacting with the graphical user interface to display individual results of each of the two or more tests performed on the point of care cartridge. In an additional aspect, when a number of test sequences have been performed on a number of individual cartridges, it may be advantageous to review these additional test sequence results. As such, there is included functionality wherein interaction with the GUI may enable one to scroll through a list of the single indicator representing the result of a testing sequence performed on each of a plurality of point of care cartridges using the point of care instrument. In another aspect to aid in patient privacy, there is a step of also preventing the display of individual test results on the graphical user interface after a time interval. In an additional aspect, there is also a step of allowing the display of individual test results on the graphical user interface after entering a security code using the graphical user interface. It is believed that the use of one or more or combinations of the above further aid in actions that may be useful or required to meet one or more of the above detailed recommendations, guidelines and requirements encountered when testing in near patient, point of care and CLIA waived environments.

### 3.3 Example 3: Rapid Confirmation of Quantity—Cartridge—Interface

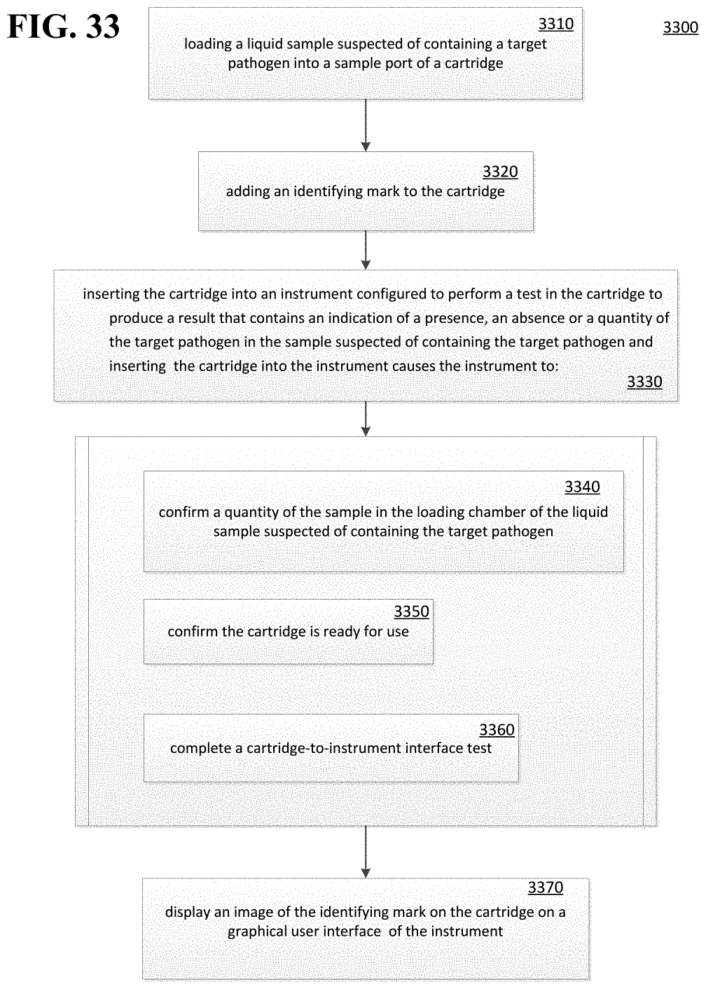

FIG. 33 is a flow chart of an exemplary method 3300 of operating an instrument for testing a sample suspected of containing a target pathogen. Embodiments of this illustrative workflow aid in accomplishing one or more of the recommendations, guidelines and requirements above. Consider, by way of example, some embodiments of this workflow method may be adapted to automatically ensure that a manufacturer's indications for use or testing sequences are performed automatically without user interaction.

The method 3300 of operating an instrument for testing a sample suspected of containing a target pathogen includes a step 3310 of loading a liquid sample suspected of containing the target pathogen into a sample port of a cartridge.

Next, there is a step 3220 of adding an identifying mark to the cartridge.

Next, at step 3330 there is a step of inserting the cartridge into an instrument configured to perform a test in the cartridge to produce a result that contains an indication of a presence, an absence or a quantity of the target pathogen in the sample suspected of containing the target pathogen.

Additionally, by performing step 3330, the act of inserting the cartridge into the instrument causes the instrument to: confirm a quantity of the liquid sample in a loading chamber of the sample suspected of containing the target pathogen (step 3340), confirm the cartridge is ready for use (step 3350), and complete a cartridge-to-instrument interface test (step 3360).

Thereafter, at step 3370, there is a step for the instrument to display an image of the identifying mark on the cartridge on a graphical user interface of the instrument.

Additionally, the method 3300 may also include a step of causing the instrument to initiate the test in the cartridge after displaying the image of the identifying mark on the graphical user interface for a predetermined time interval of less than 90 seconds.

In still another alternative, the method 3300 may also include the step of causing the instrument to eject the cartridge if the step to confirm the quantity of the sample in the loading chamber indicates an insufficient quantity of the sample or the step to confirm the cartridge is ready for use indicates the cartridge is not ready for use or the step to complete a cartridge-to-interface test indicates an unsatisfactory cartridge-to-instrument interface.

In various alternatives of the method, the liquid sample has a volume between 0.2 ml and 5 ml, inclusive, the volume of the liquid sample is between 0.5 ml and 1.5 ml, inclusive or the volume of the liquid sample is approximately 1 ml. In still other variations, the liquid sample is urine, blood, sputum, saliva, or other oral fluids. Additionally or optionally, the liquid sample is a suspension released from a swab collected from a patient.

Additional variations of the method are also possible including loading the sample further comprises sealing the sample port. Additionally, the identifying mark is handwritten or a barcode. Still further, the identifying mark identifies a patient from which the sample is acquired. The identifying mark may identify the patient by name, ID number and/or date-of-birth. In still other variations, the identifying mark further indicates a sample type. Additionally or optionally, the sample type is selected from the group consisting of urine, blood, sputum, saliva, oral fluids, and target specimen released from a genital swab, oropharyngeal swab, nasopharyngeal swab, buccal swab and rectal swab. Optionally, the identifying mark is placed in a patient label area of the cartridge.

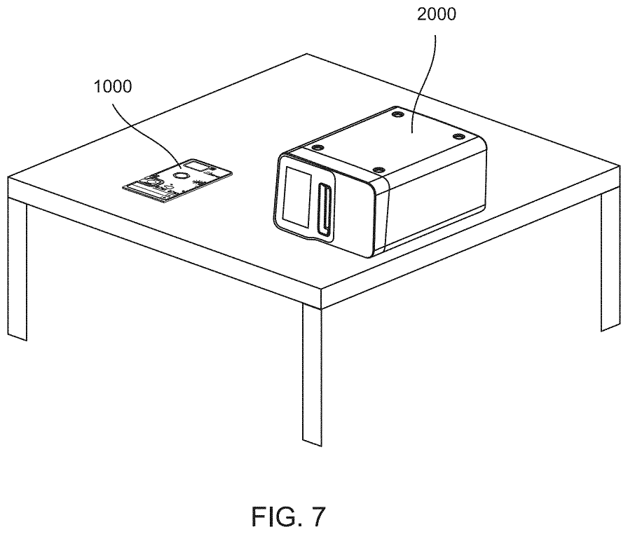

In yet another alternative of the method, the step of inserting the cartridge into the instrument comprises inserting the cartridge containing the sample into a vertically oriented loading slot of the instrument. In one aspect, loading the sample into the cartridge comprises flowing a liquid sample into the sample port, wherein the cartridge is horizontally oriented. (See. FIGS. 7, 8 and 9). The method may also include the step of canceling a testing protocol based on the image of the identifying mark on the instrument graphical user interface. In yet another alternative, the instrument graphical user interface is a touchscreen and canceling the testing protocol comprises interacting with a portion of the touchscreen. (See FIG. 13).

### 3.4 Example 4: Fault Detection with Auto Ejection

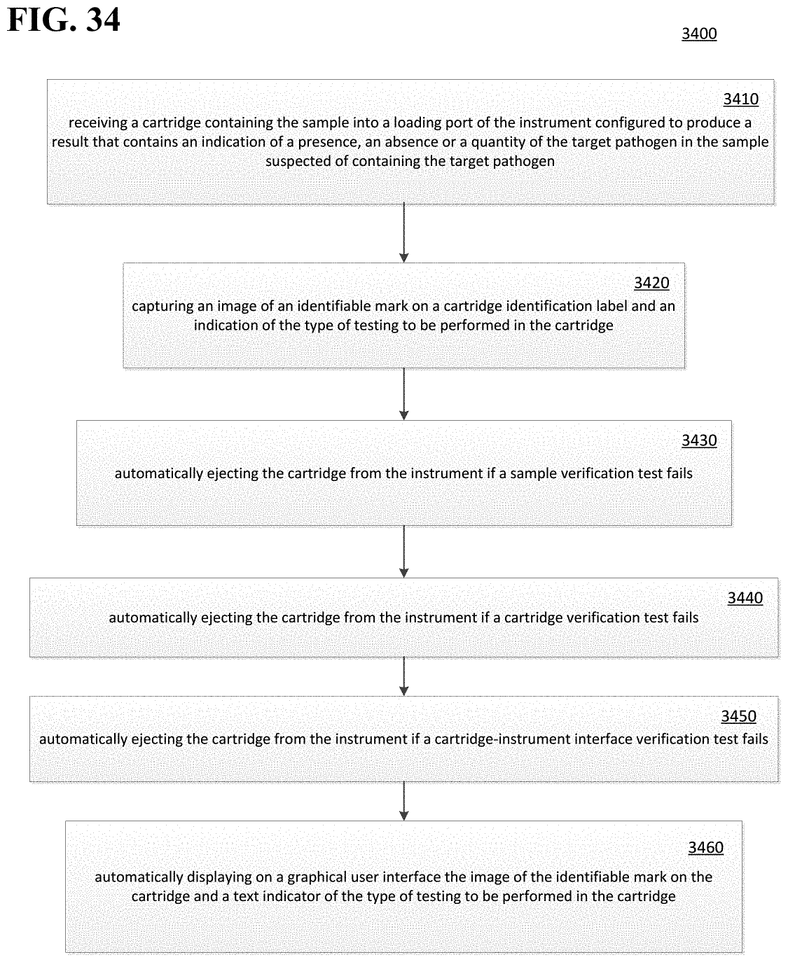

FIG. 34 is a flow chart of an exemplary method 3400 of operating an instrument for testing a sample suspected of containing a target pathogen. Embodiments of this illustrative workflow aid in accomplishing one or more of the recommendations, guidelines and requirements above. Advantageously, embodiments of this exemplary method may aid the user by automatically ejecting a cartridge when any of a range of faults or verification tests fall outside of acceptable parameters. As with other aspects of the inventive workflow detailed above, embodiments of this inventive method may aid the user in automatically following a manufacturers' instructions via the automatic performance of cartridge, sample and instrument specific tests or verifications.

In an exemplary implementation of the method 3400, there is a step 3410 of receiving a cartridge containing the sample into an opening of the instrument configured to produce a result that contains an indication of a presence, an absence or a quantity of the target pathogen in the sample suspected of containing the target pathogen. (see e.g., FIG. 12)

Next, there is a step 3420 of capturing an image of an identifiable mark on a cartridge identification label and an indication of the type of testing to be performed in the cartridge.

Next, there are one or more verification steps automatically performed by the instrument based on the specific requirements of a manufacturer's instrument, integrated cartridge or sample type. In one exemplary series of verification steps, there is a step 3430 of automatically ejecting the cartridge from the instrument if a sample verification test fails. There is also a step 3440 of automatically ejecting the cartridge from the instrument if a cartridge verification test fails. There is also a step 3450 of automatically ejecting the cartridge from the instrument if a cartridge-instrument interface verification test fails. Other combinations of more or fewer verifications steps are possible based on the specific configuration of an integrated cartridge, a point of care instrument of sample type as discussed herein. In additional aspects, any number and combination of verification tests may be performed without the user's knowledge or that require further interaction with the instrument. Steps such as these not only provide the benefit of being easy to operate for untrained or limited trained instrument operators, but a manufacturer may provide appropriate verification testing sequences as needed to conform with their approved indications for use or CLIA waived testing protocols.

Next, there is also a step 3460 of automatically displaying on a graphical user interface the image of the identifiable mark on the cartridge and a text indicator of the type of testing to be performed in the cartridge. An automatic step such as this one is also useful in achieving one or more of the recommendations, guidelines and requirements of sample—patient identification. Ease of use is also enabled as described herein in that a user may readily self-select virtually an identifiable mark suited to their operating environment or clinical practice.

In one aspect of performing the method, the opening is a vertically oriented loading slot. Additionally, the instrument is configured to maintain the cartridge in a vertical orientation during testing in the cartridge. These aspects are appreciated by reference to FIGS. 9, 11 and 12 as well as the cartridge loading details of FIGS. 25 and 26A.

In an additional aspect of the method, capturing an indication of the type of testing to be performed on the cartridge comprises parsing a machine-readable barcode. Still further, the step of capturing the image of the identifiable mark occurs within an interior space of the instrument. These aspects may be appreciated by reference to the views of a cartridge and machine readable markings visible in FIGS. 2 and 14 along with the cartridge-instrument arrangements shown in FIGS. 24, 29A and 29B.

In an additional aspect, the method includes a step of permitting a user to manually halt testing within a set time period of displaying the image of the identifiable mark on the graphical user interface. In one embodiment, the set time period is ten seconds.

Still additional embodiments of the method include a step of ejecting the cartridge responsive to receiving a termination command from the user within the set time period. In another aspect, there is also a step of initiating a diagnostic assay protocol on the cartridge after elapse of the set time period without receiving a termination command from the user.

In yet another alternative, the method includes a step of initiating a diagnostic assay protocol on the cartridge to generate a test result. Thereafter, there is a step of automatically displaying on the graphical user interface the result that contains an indication of a presence, an absence or a quantity of the target pathogen in the sample suspected of containing the target pathogen. This step provides an additional example of the ease of use of the inventive workflows using automatically initiating an assay protocol and displaying results on the easy to view GUI on the front panel of the instrument.

### 3.5 Example 5: Auto Confirmation without User Interaction

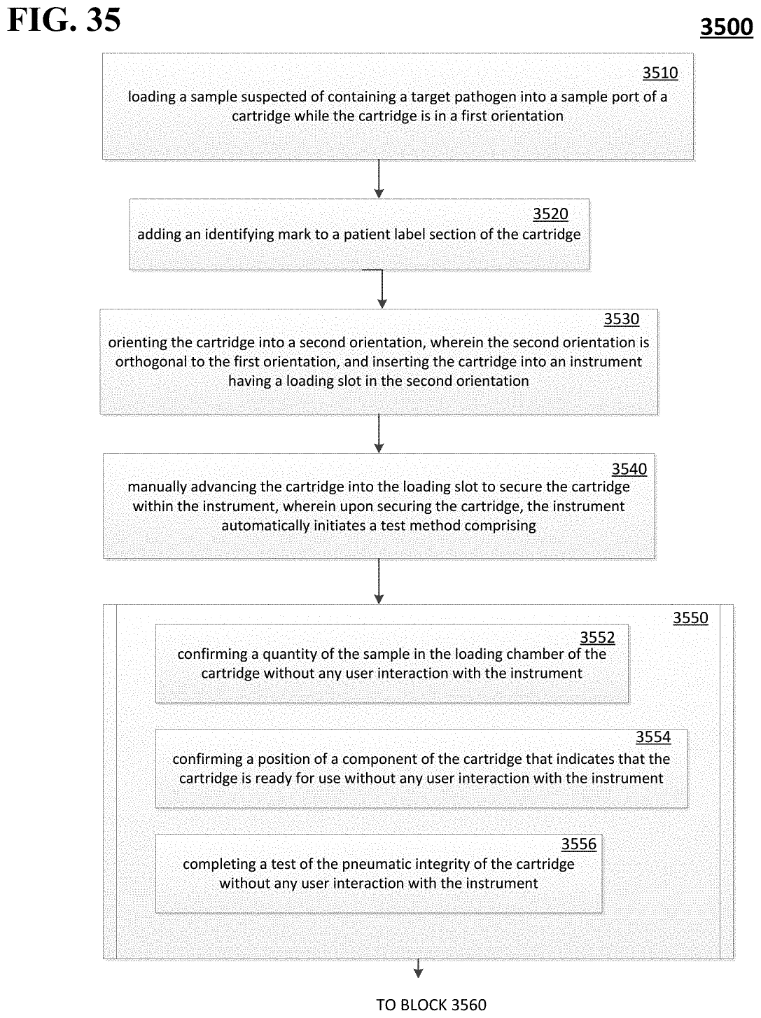

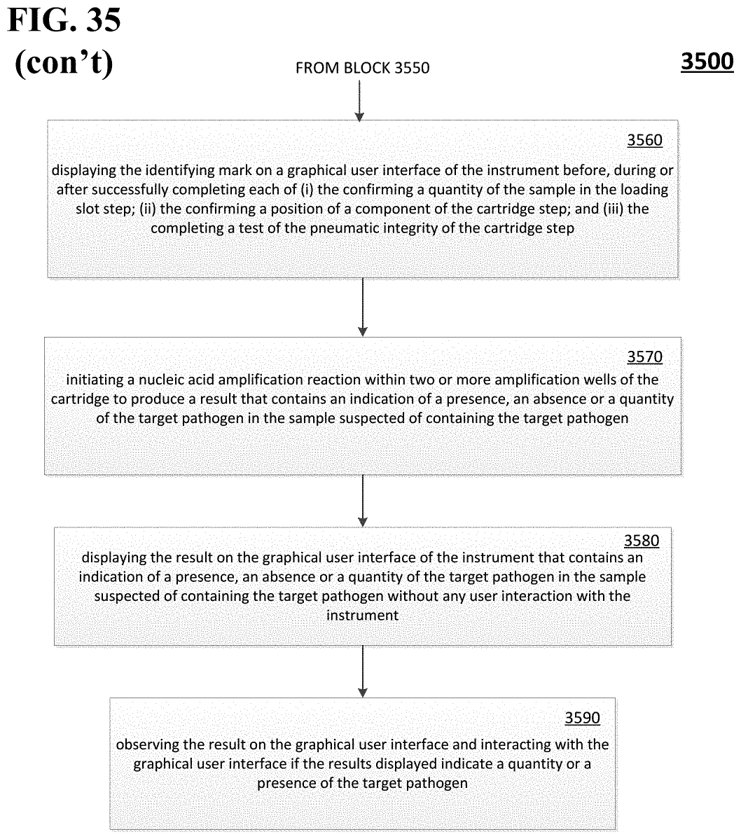

FIG. 35 is a flow chart of an exemplary method 3500 of operating an instrument for testing a sample suspected of containing a target pathogen. Embodiments of this illustrative workflow aid in accomplishing one or more of the recommendations, guidelines and requirements above. Advantageously, embodiments of this exemplary method may aid the user by automatically confirming a number of different parameters that, based on a specific sample, cartridge or instrument requirement, must be met in order to obtain a reliable result. Importantly, the performance of such confirming actions recommended by a particular manufacturer that appropriate to sample, cartridge or instrument indications for use for that manufacturer aid in time savings by ensuring up front at initial loading or soon thereafter that such parameters are acceptable. Automatically performing the appropriate confirmation steps alleviates such a requirement from the user and, if implemented properly, may fulfill even if only partially, some or all requirements and guidelines related to following manufacturer instructions and indications for use. As with other aspects of the inventive workflow detailed above, embodiments of this inventive method may aid the user in automatically following a manufacturers' instructions via the automatic performance of cartridge, sample and instrument specific tests or verifications.

The method 3500 of operating an instrument for testing a sample suspected of containing a target pathogen includes a step 3510 of loading the sample suspected of containing the target pathogen into a sample port of a cartridge while the cartridge is in a first orientation. By way of example, a first orientation may be appreciated with reference to FIG. 7.

Next, the method includes a step 3520 for adding an identifying mark to a patient label section of the cartridge.

Next, the method includes a step 3530 of orienting the cartridge into a second orientation, wherein the second orientation is orthogonal to the first orientation, and inserting the cartridge into an instrument having a loading slot in the second orientation. By way of example, this step may be appreciated by reference to FIGS. 8 and 12.

Next, the method includes a step 3540 of manually advancing the cartridge into the loading slot to secure the cartridge within the instrument. For example, a cartridge may be engaged within an appropriate instruct as shown, for example, in the views of FIG. 26A

Next, at step 3550, upon securing the cartridge, the instrument automatically initiates a test method including a series of steps. One exemplary test method includes confirming a quantity of the sample in a loading chamber of the cartridge without any user interaction with the instrument (step 3552). Additionally or optionally, the test method includes confirming a position of a component of the cartridge that indicates that the cartridge is ready for use without any user interaction with the instrument (step 3554). Still further, the testing method includes completing a test of the pneumatic integrity of the cartridge without any user interaction with the instrument (step 3556).

Thereafter, the method continues with a step 3560 of displaying the identifying mark on a graphical user interface of the instrument before, during, or after successfully completing each of the confirming a quantity of the sample in the loading chamber step 3552, the confirming a position of a component of the cartridge step 3554 and the completing a test of the pneumatic integrity of the cartridge step 3556. In furtherance of objectives related to ease of use, the GUI may also provide other status based information to a user such as illustrated in FIG. 14 (indicating one or more automatic confirmation operations is in progress).

Thereafter, the method continues with a step 3570 of initiating a nucleic acid amplification reaction within two or more amplification wells of the cartridge to produce a result that contains an indication of a presence, an absence or a quantity of the target pathogen in the sample suspected of containing the target pathogen. See, for example, FIG. 15 with a GUI display indicating that one or more or a combination of sample, cartridge or instrument based confirmation steps are completed and testing protocol has been automatically initiated.

Next, at step 3580, the method continues by displaying the result on the graphical user interface that contains an indication of a presence, an absence or a quantity of the target pathogen in the sample suspected of containing the target pathogen without any user interaction with the instrument. (See FIG. 17).

Next, there is a step 3590 for observing the result on the graphical user interface and interacting with the graphical user interface if the results displayed indicate a quantity or a presence of the target pathogen. FIGS. 19A and 19B illustrate representative GUI displays of user interaction with the GUI allowing for review of results as well as for changing the display of results including scrolling through or obtaining additional details of a specific result of interest.

In one alternative, the first orientation is horizontal and the second orientation is vertical. In one aspect, the first orientation the cartridge height axis of the cartridge is normal to a work surface supporting the cartridge during the loading step or the adding step or supporting the instrument. In one alternative, the second orientation the cartridge height axis of the cartridge is parallel to a work surface supporting the cartridge during the loading step or the adding step or supporting the instrument. Additionally or optionally, the second orientation the cartridge length axis of the cartridge also is parallel to the work surface supporting the cartridge during the loading step and the adding step. In another aspect, in the second orientation a cartridge length axis is also parallel to the longest axis of the instrument. The cartridge length axis of an instrument may be the one normal to the rear wall of the instrument and/or normal to the front face of the instrument having the GUI and loading slot. If the front face is slanted, then the longest axis would be normal to a back panel of the instrument. In other words, the cartridge length axis may be determined from a front panel or rear panel of the instrument depending upon which one is flat and most nearly normal to the work surface.

In additional aspects, the second orientation the cartridge width axis of the cartridge is normal to the base of the instrument. In additional alternative, the second orientation the cartridge height axis of the cartridge is parallel to a rear wall of the instrument. In yet another alternative, there is an additional step of moving the cartridge into the second orientation by rotating the cartridge about the cartridge length axis. FIGS. 24 and 25, for example, provide additional context for understanding additional aspects of the relationship and orientation between the cartridge and instrument.

### 3.6 Example 6: Screening Application with Ease of Use Workflow

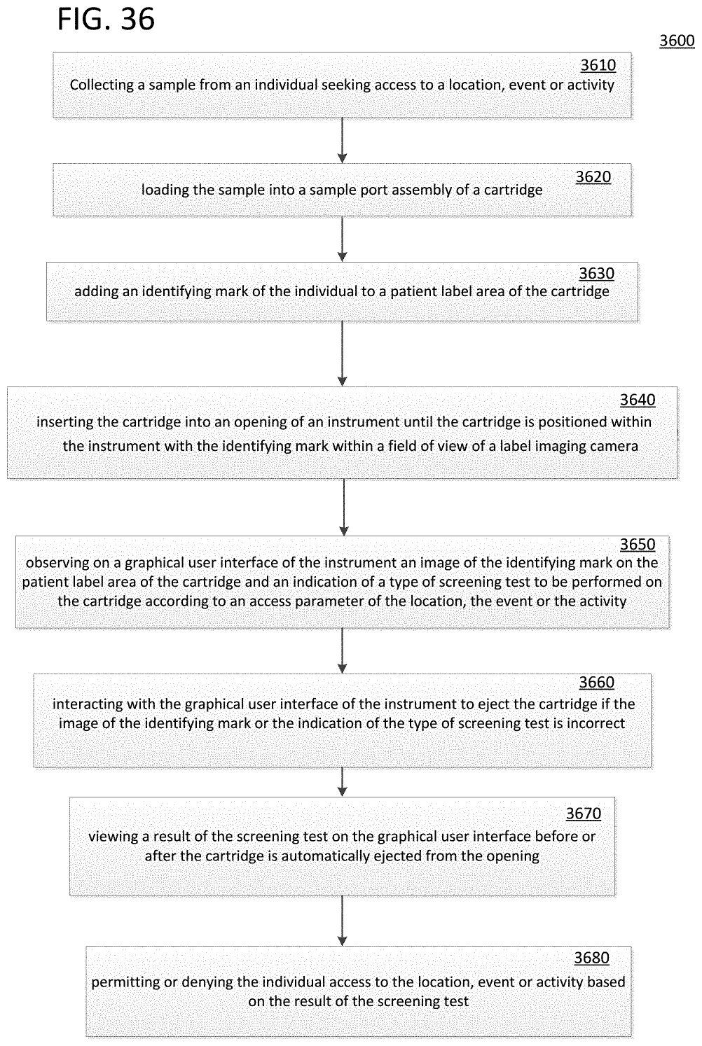

FIG. 36 is a flow chart of an exemplary method 3600 of operating an instrument for testing a sample suspected of containing a target pathogen from an individual seeking access to a location, event or activity. Embodiments of this illustrative workflow aid in accomplishing one or more of the recommendations, guidelines and requirements above as applicable to the screening use case and environment. The ease of use and reliable results coupled with minimal user involvement render the various embodiments described herein well suited for screening applications. Screening individuals for access to locations, events or activities may be used in response to any public health concern or outbreak including, for example and not limitation, an influenza pandemic, an epidemic or pandemic including 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19) as well as human coronaviruses 229E, NL63, OC43, and HKU1 or any other variety of human coronaviruses.

The method 3600 begins by collecting a sample from an individual seeking access to a location, event or activity (step 3610). The sample collected may be any of those described herein identified for the specific screening function being performed.

Next, the sample is loaded into a sample port of a cartridge (step 3620).

An identifying mark is added to the patient label area of the cartridge (step 3630). The identifying mark may be specifically selected to include one or more pieces of information identifying the patient who provided the sample. Examples of additional information may include passport or travel information, seat on a mode of transportation, seat in an event, lodging or hotel information.

The cartridge is inserted into an opening of an instrument until the cartridge is positioned within the instrument with the identifying mark within a field of view of a label imaging camera (step 3640).

Next, there is a step of observing on a graphical user interface of the instrument an image of the identifying mark on the patient label area of the cartridge and an indication of a type of screening test to be performed on the cartridge according to an access parameter of the location, the event or the activity (step 3650). Additionally or optionally, this and other steps may be performed by the person seeking access or a worker assigned to supervise or conduct the screening evaluations.

There is also a step of interacting with the graphical user interface of the instrument to eject the cartridge if the image of the identifying mark or the indication of the type of screening test is incorrect (step 3660).

Next, there is a step of viewing a result of the screening on the graphical user interface before or after the cartridge is automatically ejected from the opening of the instrument (step 3670).

Finally, there is a step of permitting or denying the individual access to the location, event or activity based on the result of the screening test (step 3680).

Embodiments of the screening method 3600 may be used for controlling access of individuals to airports, ship terminals, train stations and other transportation hubs, border crossings or checkpoints. The screening method may be employed for controlling entry into local, state or federal buildings or service points, schools, education or training centers, as well as worksite, office or building access. In one alternative embodiment, step 3670 is performed by an individual responsible for conducting the screening method. The person conducting the screening may inform the individual of the results and permit access if the individual passed the screening test. Additionally, if the individual did not pass the screening test, the individual may be directed to an exit or to additional screening station.

### Summary of Advantages and Benefits Provided Users by the Embodiments of the Inventive Workflow

In view of the examples and various alternatives described above, those of ordinary skill in the fields of integrated cartridge and point of care instrument design will appreciate that the inventive workflow enables one or more of the recommendations, guidelines and requirements detailed above. Still further, the various details of the embodiments of the cartridge, instrument and workflows above provide numerous advantages related to achieving a simple point of care workflow including, by way of example and not limitation: a CT/NG Assay in a cartridge for use with a complementary designed instrument which may only be inserted in a desired alignment (FIGS. 27A-28B); enables basic, non-technique-dependent specimen handling, including handling direct unprocessed samples from vaginal swabs or urine specimens (FIG. 5); the operator applies sample custody information directly to the cartridge in the label area provided using any form of identifying marking (FIGS. 4A and 4B); the cartridge contains all reagents; requiring no reagent handling (FIG. 3); the sample loading port is large and clearly marked (FIGS. 2, 3, 5 and 6); the closure lid is large and easy to snap closed (FIGS. 5 and 6); the operator designated sample identification is written and/or applied directly to the cartridge accepting the sample and is then imaged automatically without user interaction by the instrument using a cartridge label imaging camera and included alongside the reporting of test results; the assay cartridge has alignment and keying features for ensuring correct orientation when loaded into the instrument (FIGS. 27A-28B); the instrument performs all sample preparation steps and completes all analysis automatically and eliminates subjectivity of any visual readings by the end-user (FIGS. 31-36); simple to read test results are presented prominently in the GUI as positive, negative or invalid for each indication and there is no user interpretation involved; the GUI is designed for ease of use and uses a large color display with easy to read messages; Error messages are unambiguous and include easy to interpret solutions (see FIGS. 2-23); operation and interaction with the instrument does not require troubleshooting or interpretation error codes; instrument maintenance is limited to wiping of the external surfaces; the instrument requires no calibration to be performed by the user; the instrument contains no serviceable parts and the instrument is to be returned to the manufacturer if maintenance is required; and the test procedure and information provided on the GUI is written at a 7^{th} grade comprehension level.

In view of the examples and various alternatives described above, those of ordinary skill in the fields of integrated cartridge and point of care instrument design will additionally appreciate that the various details of the embodiments of the workflows above provide numerous advantages related to achieving a point of care workflow which is properly characterized as providing an insignificant risk of an erroneous result including, by way of example and not limitation: the patient label area on the cartridge must contain some degree of writing; a blank label on the cartridge is not accepted, as the operator would not be able to determine the sample custody for a blank label region on a cartridge; test results displayed by the system include the picture of the sample custody information or the identifying mark placed in or on the patient label area; test reports generated by the system include the captured image of the identifying mark or a picture of the sample custody information; the cartridge contains sample metering so that the instrument operation is not reliant on an accurate or precise sample pipetting by the user; the instrument verifies the operator loaded the correct volume in the cartridge and will reject the cartridge if inadequate sample is loaded; the cartridge is tolerant to an expected amount sample overage; the cartridge lid is irreversibly closed when the operator snaps it closed which prevents the operator from inadvertently loading sample into a previously used cartridge; the instrument prevents from running a previously used cartridge by examining the valve position or other appropriate cartridge confirmation action; the instrument verifies that the cartridge micro-fluidic valve has properly dropped into place from the shipping position or other cartridge specific verification action; the instrument verifies whether there is a cartridge in the instrument upon power up; the instrument irreversibly faults and locks the cartridge into the instrument if it detects that fluid has leaked during amplification so as to prevent amplicon from getting into the instrument enclosure, potentially contaminating an operator's laboratory or workstation potentially increasing the risk of obtaining false positive results; the instrument utilizes a sample processing control, Human Beta Actin, to ensure that a human sample was properly loaded into the sample port and then that nucleic acid properly extracted and amplified so as to ensure sample adequacy, sample extraction effectiveness, and amplification integrity; the cartridge includes duplicate data matrix codes that encode the manufacturing bar code to increase reliability with one matrix code positioned so that it is read along with the sample custody label marking when the cartridge is loaded; the instrument requires external controls to be run and pass during initial setup; a failed external control at any time puts the instrument in Quarantine Mode and requires a Pass test result for the same external control type to return to normal operation where Patient samples can be tested (FIG. 20).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of operating an instrument for testing a sample suspected of containing a target pathogen, comprising:
loading the sample suspected of containing the target pathogen into a sample port assembly of a cartridge;
adding an identifying mark to a patient label area of the cartridge;
inserting the cartridge into an opening of the instrument until the cartridge is positioned within the instrument with the identifying mark within a field of view of a label imaging camera;
observing on a graphical user interface of the instrument an indication of a type of test to be performed on the cartridge and an image of the identifying mark on the patient label area of the cartridge;
interacting with the graphical user interface of the instrument to eject the cartridge if the image of the identifying mark or the indication of the type of test is incorrect; and
removing the cartridge from the opening of the instrument after the cartridge is automatically ejected from the opening.

2. (canceled)

3. The method of claim 1 further comprising: observing an image of the identifying mark and an indication of a presence, an absence or a quantity of the target pathogen in the sample on the graphical user interface before or during the removing step.

4. The method of claim 1 further comprising automatically initiating a testing protocol when a predetermined time period has elapsed after completing the inserting the cartridge step.

5. The method of claim 4 wherein the predetermined time period is less than 2 minutes, less than one minute, less than 30 seconds, or less than 10 seconds.

6. (canceled)

7. The method of claim 1 wherein the adding an identifying mark step further comprising: affixing a printed label or a printed machine readable label to the patient label area.

8. (canceled)

9. The method of claim 1 wherein the adding an identifying mark step further comprising: handwriting sample identifying information in the patient label area.

10.-11. (canceled)

12. The method of claim 1 further comprising initiating at least one cartridge verification test without any user interaction with the instrument after performing the inserting the cartridge step.

13. The method of claim 1 further comprising: observing on the graphical user interface of the instrument the identifying mark without touching the graphical user interface of the instrument or performing any other user interaction to contact the instrument while or prior to performing the removing the cartridge step.

14. The method of claim 1, wherein after the inserting the cartridge step the instrument automatically performs a nucleic acid amplification process to produce a result that contains an indication of a presence, an absence or a quantity of the target pathogen in the sample suspected of containing the target pathogen without touching the graphical user interface of the instrument or otherwise interacting with the instrument.

15. The method of claim 1 further comprising: observing an image, an icon or a glyph on the graphical user interface indicating a result of a testing protocol performed on the sample in the cartridge during, after, or before the removing step.

16. The method of claim 15 wherein the step of observing the image, the icon, or the glyph on the graphical user display is performed less than 60 min., 25 min, less than 20 min, less than 15 min, or less than 10 min after performing the inserting a cartridge into the instrument step.

17. (canceled)

18. A method of testing a sample suspected of containing a target pathogen, comprising:
inserting the sample suspected of containing the target pathogen into a point of care cartridge;
placing an identifying mark on a patient label section of the point of care cartridge;
inserting the point of care cartridge into an opening of a point of care instrument until the patient label section of the point of care cartridge is within a field of view of a label imaging camera within an interior portion of the point of care instrument;
observing on a graphical user interface of the point of care instrument an image of the patient label section captured by the label imaging camera; and
performing only a single interaction with the point of care instrument to observe on the graphical user interface, adjacent to the image of the patient label section, a single indicator representing a result of a testing sequence indicating a presence of the target pathogen, an absence of the target pathogen, or a quantity of the target pathogen in the sample.

19. The method of claim 18 the placing step further comprising handwriting on the patient label section to identify the sample.

20.-21. (canceled)

22. The method of claim 18 wherein a time delay of less than 15 minutes separates the observing step from the performing step.

23. (canceled)

24. The method of claim 18 wherein after the inserting step the point of care cartridge is substantially within the interior of the point of care instrument.

25. The method of claim 18 wherein the performing only a single interaction step is undertaken after observing that the point of care cartridge is ejected from the point of care instrument.

26. The method of claim 18 wherein the single indicator represents a positive test result or a negative test result.

27. The method of claim 26 wherein the single indicator for the positive test result appears in red in the graphical user interface and the single indicator for the negative test result appears in green in the graphical user interface.

28. The method of claim 27 wherein the single indicator is an image, an icon, or glyph.

29. (canceled)

30. The method of claim 18 wherein the single indicator representing a result further comprises an image, an icon, or a glyph for a presence of the pathogen or an absence of the pathogen.

31. The method of claim 18 wherein the single indicator represents a result for two or more tests performed on the point of care cartridge.

32. The method of claim 31 wherein the single indicator represents a negative presence of all target pathogens from the two or more tests or the single indicator represents a positive presence of at least one target pathogen from the two or more tests.

33. The method of claim 31 further comprising interacting with the graphical user interface to display individual results of each of the two or more tests performed on the point of care cartridge.

34. The method of claim 18 further comprising performing the inserting the sample step, the placing the identifying mark step, the inserting the point of care cartridge step, the observing on the graphical user interface step and the performing only the single interaction with the point of care instrument step on each one of a plurality of point of care cartridges to produce a plurality of the single indicator representing the result of the testing sequence performed on each one of a plurality of the point of care cartridges.

35. The method of claim 34 further comprising interacting with the graphical user interface to scroll through the plurality of the single indictor representing the result of the testing sequence performed on each one of the plurality of the point of care cartridges.

36. The method of claim 33 further comprising preventing the display of individual test results on the graphical user interface after a time interval.

37. The method of claim 36 further comprising allowing the display of individual test results on the graphical user interface after entering a security code using the graphical user interface.

38.-75. (canceled)
